SEQUENCE LISTING (146 sequences)

SEQ ID NO: 1 - protein, 7 aa, Simian virus 40
PKKKRKV

SEQ ID NO: 2 - protein, 16 aa, Unknown
  Description of Unknown nucleoplasmin nuclear localization signal sequence
KRPAATKKAGQAKKKK

SEQ ID NO: 3 - protein, 9 aa, Unknown
  Description of Unknown c-myc nuclear localization signal sequence
PAAKRVKLD

SEQ ID NO: 4 - protein, 11 aa, Unknown
  Description of Unknown c-myc nuclear localization signal sequence
RQRRNELKRSP

SEQ ID NO: 5 - protein, 38 aa, Unknown
  Description of Unknown hRNPA1 M9 nuclear localization signal sequence
NQSSNFGPMKGGNFGGRSSGPYGGGGQYFAKPRNQGGY

SEQ ID NO: 6 - protein, 42 aa, Unknown
  Description of Unknown IBB domain from importin-alpha nuclear localization signal sequence
RMRIZFKNKGKDTAELRRRRVEVSVELRKAKKDEQILKRRNV

SEQ ID NO: 7 - protein, 8 aa, Unknown
  Description of Unknown myoma T protein nuclear localization signal sequence
VSRKRPRP

SEQ ID NO: 8 - protein, 8 aa, Unknown
  Description of Unknown myoma T protein nuclear localization signal sequence
PPKKARED

SEQ ID NO: 9 - protein, 8 aa, Homo sapiens
PQPKKKPL

SEQ ID NO: 10 - protein, 12 aa, Mus sp.
SALIKKKKKMAP

SEQ ID NO: 11 - protein, 5 aa, Influenza virus
DRLRR

SEQ ID NO: 12 - protein, 7 aa, Influenza virus
PKQKKRK

SEQ ID NO: 13 - protein, 10 aa, Hepatitis delta virus
RKLKKKIKKL

SEQ ID NO: 14 - protein, 10 aa, Mus sp.
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - DNA, 27 bp, Artificial Sequence
  Description of Artificial Sequence Synthetic oligonucleotide
  modified_base (1)..(20): a, g, t or c
  modified_base (21)..(22): a, c, t, g, unknown or other
nnnnnnnnnnnnnnnnnnnnnnagaaw

SEQ ID NO: 18 - DNA, 19 bp, Artificial Sequence
  Description of Artificial Sequence Synthetic oligonucleotide
  modified_base (1)..(12): a, g, t or c
  modified_base (13)..(14): a, c, t, g, unknown or other
nnnnnnnnnnnnnnagaaw

SEQ ID NO: 19 - DNA, 27 bp, Artificial Sequence
  Description of Artificial Sequence Synthetic oligonucleotide
  modified_base (1)..(20): a, g, t or c
  modified_base (21)..(22): a, c, t, g, unknown or other
nnnnnnnnnnnnnnnnnnnnnnagaaw

SEQ ID NO: 20 - DNA, 18 bp, Artificial Sequence
  Description of Artificial Sequence Synthetic oligonucleotide
  modified_base (1)..(11): a, g, t or c
  modified_base (12)..(13): a, c, t, g, unknown or other
nnnnnnnnnnnnnagaaw

SEQ ID NO: 21 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 22 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaathcagaagctacaaagataaggcttcatgccgaaatcaacaccctgtcattttatggcagggtgttttcgttatttaatttttt

SEQ ID NO: 23 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 24 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 25 - DNA, 87 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtttttttt

SEQ ID NO: 26 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 27 - DNA, 21 bp, Artificial Sequence
  Description of Artificial Sequence Synthetic oligonucleotide
gctctgtgactagtcacagag

SEQ ID NO: 28 - DNA, 19 bp, Artificial Sequence
  Description of Artificial Sequence Synthetic oligonucleotide
gcggaacacatgtgttccg

SEQ ID NO: 29 - DNA, 20 bp, Artificial Sequence
  Description of Artificial Sequence Synthetic oligonucleotide
tcgatcgtcgcgacgatcga

SEQ ID NO: 30 - DNA, 20 bp, Artificial Sequence
  Description of Artificial Sequence Synthetic oligonucleotide
cgacgatcgatcgatcgtcg

SEQ ID NO: 31 - DNA, 60 bp, Artificial Sequence
  Description of Artificial Sequence Synthetic oligonucleotide
  modified_base (20)..(38): a, c, t, g, unknown or other
tggaaaggacgaaacaccgnnnnnnnnnnnnnnnnnnngtttaagagctatgctggaaac

SEQ ID NO: 32 - DNA, 60 bp, Artificial Sequence
  Description of Artificial Sequence Synthetic oligonucleotide
  modified_base (19)..(38): a, c, t, g, unknown or other
ggaaaggacgaaacaccgnnnnnnnnnnnnnnnnnnnngtttaagagctatgctggaaac

SEQ ID NO: 33 - DNA, 60 bp, Artificial Sequence
  Description of Artificial Sequence Synthetic oligonucleotide
  modified_base (20)..(37): a, c, t, g, unknown or other
tggaaaggacgaaacaccgnnnnnnnnnnnnnnnnnngtttaagagctatgctggaaaca

SEQ ID NO: 34 - DNA, 264 bp, Artificial Sequence
  Description of Artificial Sequence Synthetic polynucleotide
  modified_base (123)..(142): a, c, t, g, unknown or other
  misc_feature (123)..(142): This region may encompass 18 to 20 nucleotides wherein some positions may be absent
atttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnngtttaagagctatgctggaaacagcatagcaagtttaaataaggctagtccgttatcaacttgaaaaagtggcaccgagtcggtgcttttttgttttagagctagaaatagcaagttaaaat

SEQ ID NO: 35 - DNA, 500 bp, Artificial Sequence
  Description of Artificial Sequence Synthetic polynucleotide
  modified_base (266)..(285): a, c, t, g, unknown or other
  misc_feature (266)..(285): This region may encompass 18 to 20 nucleotides wherein some positions may be absent
gagtattacggcatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngtttaagagctatgctggaaacagcatagcaagtttaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacggccgcttcgagcagacatgataagatacattga

SEQ ID NO: 36 - DNA, 20 bp, Artificial Sequence
  Description of Artificial Sequence Synthetic oligonucleotide
gtgagcaagggcgaggagct

SEQ ID NO: 37 - DNA, 20 bp, Artificial Sequence
  Description of Artificial Sequence Synthetic oligonucleotide
tgaggagtgaattgcggccg

SEQ ID NO: 38 - DNA, 215 bp, Artificial Sequence
  Description of Artificial Sequence Synthetic polynucleotide
ccttgtgggtctgtttgaggacaccaacctgtgcgccatccacgccaagcgtgtcaccatcatgcccaaggacatccaactggcccgccgcatccgcggggagagggcgtaattgtacagttccgtacagttctacggaattaaccaaaggctcttttcagagccacacacgatttcaagtaaaatggctgtaatttactccatccttaaacgaa

SEQ ID NO: 39 - DNA, 21 bp, Artificial Sequence
  Description of Artificial Sequence Synthetic oligonucleotide
gctctgtgactagtcacagag

SEQ ID NO: 40 - DNA, 19 bp, Artificial Sequence
  Description of Artificial Sequence Synthetic oligonucleotide
gcggaacacatgtgttccg

SEQ ID NO: 41 - DNA, 21 bp, Artificial Sequence
  Description of Artificial Sequence Synthetic oligonucleotide
gtcgatcgtcgcgacgatcga

SEQ ID NO: 42 - DNA, 21 bp, Artificial Sequence
  Description of Artificial Sequence Synthetic oligonucleotide
gcgacgatcgatcgatcgtcg

SEQ ID NO: 43 - DNA, 20 bp, Artificial Sequence
  Description of Artificial Sequence Synthetic oligonucleotide
gctgaagcactgcacgccgt

SEQ ID NO: 44 - DNA, 20 bp, Artificial Sequence
  Description of Artificial Sequence Synthetic oligonucleotide
gttaattccgtagaactgta

SEQ ID NO: 45 - DNA, 21 bp, Artificial Sequence
  Description of Artificial Sequence Synthetic oligonucleotide
gtatgagacttacgcaacatc

SEQ ID NO: 46 - DNA, 60 bp, Artificial Sequence
  Description of Artificial Sequence Synthetic oligonucleotide
taacttgaaagtatttcgatttcttggctttatatatcttgtggaaaggacgaaacaccg

SEQ ID NO: 47 - DNA, 60 bp, Artificial Sequence
  Description of Artificial Sequence Synthetic oligonucleotide
gttgataacggactagccttatttaaacttgctatgctgtttccagcatagctcttaaac

SEQ ID NO: 48 - DNA, 90 bp, Artificial Sequence
  Description of Artificial Sequence Synthetic oligonucleotide
atttcttgggtagtttgcagttttaaaattatgttttaaaatggactatcatatgcttaccgtaacttgaaagtatttcgatttcttggc

SEQ ID NO: 49 - DNA, 90 bp, Artificial Sequence
  Description of Artificial Sequence Synthetic oligonucleotide
attttaacttgctatttctagctctaaaacaaaaaagcaccgactcggtgccactttttcaagttgataacggactagccttatttaaac

SEQ ID NO: 50 - DNA, 20 bp, Artificial Sequence
  Description of Artificial Sequence Synthetic oligonucleotide
gggcgaggagctgttcaccg

SEQ ID NO: 51 - DNA, 20 bp, Artificial Sequence
  Description of Artificial Sequence Synthetic oligonucleotide
gagctggacggcgacgtaaa

SEQ ID NO: 52 - DNA, 20 bp, Artificial Sequence
  Description of Artificial Sequence Synthetic oligonucleotide
gctgaagcactgcacgccgt

SEQ ID NO: 53 - DNA, 20 bp, Artificial Sequence
  Description of Artificial Sequence Synthetic oligonucleotide
gttaattccgtagaactgta

SEQ ID NO: 54 - DNA, 21 bp, Artificial Sequence
  Description of Artificial Sequence Synthetic oligonucleotide
gtatgagacttacgcaacatc

SEQ ID NO: 55 - DNA, 20 bp, Artificial Sequence
  Description of Artificial Sequence Synthetic oligonucleotide
gaactccttgatgacgtcct

SEQ ID NO: 56 - DNA, 20 bp, Artificial Sequence
  Description of Artificial Sequence Synthetic oligonucleotide
gccaagctgaaggtgaccaa

SEQ ID NO: 57 - DNA, 20 bp, Artificial Sequence
  Description of Artificial Sequence Synthetic oligonucleotide
gcgctaagtaaacagtgagt

SEQ ID NO: 58 - DNA, 20 bp, Artificial Sequence
  Description of Artificial Sequence Synthetic oligonucleotide
gtgatggcccagcacatgga

SEQ ID NO: 59 - DNA, 20 bp, Artificial Sequence
  Description of Artificial Sequence Synthetic oligonucleotide
ggccagccatgctggtaggc

SEQ ID NO: 60 - DNA, 21 bp, Artificial Sequence
  Description of Artificial Sequence Synthetic oligonucleotide
gcctgagctggtgctaccaag

SEQ ID NO: 61 - DNA, 21 bp, Artificial Sequence
  Description of Artificial Sequence Synthetic oligonucleotide
gcacttttaaatcccacgtag

SEQ ID NO: 62 - DNA, 20 bp, Artificial Sequence
  Description of Artificial Sequence Synthetic oligonucleotide
gtttcctaaccacaggaggg

SEQ ID NO: 63 - DNA, 20 bp, Artificial Sequence
  Description of Artificial Sequence Synthetic oligonucleotide
gctcagaggcgagaattctc

SEQ ID NO: 64 - DNA, 20 bp, Artificial Sequence
  Description of Artificial Sequence Synthetic oligonucleotide
gtgagggctggactgcgaac

SEQ ID NO: 65 - DNA, 21 bp, Artificial Sequence
  Description of Artificial Sequence Synthetic oligonucleotide
gagatggggtacttctcatcc

SEQ ID NO: 66 - DNA, 20 bp, Artificial Sequence
  Description of Artificial Sequence Synthetic oligonucleotide
gaatgaacaaatgaagaaaa

SEQ ID NO: 67 - DNA, 23 bp, Artificial Sequence
  Description of Artificial Sequence Synthetic oligonucleotide
tgagtattacggcatgtgagggc

SEQ ID NO: 68 - DNA, 27 bp, Artificial Sequence
  Description of Artificial Sequence Synthetic oligonucleotide
tcaatgtatcttatcatgtctgctcga

SEQ ID NO: 69 - DNA, 60 bp, Artificial Sequence
  Description of Artificial Sequence Synthetic oligonucleotide
ggacatccaactggcccgccgcatccgcggggagagggcggtgagcaagggcgaggagct

SEQ ID NO: 70 - DNA, 60 bp, Artificial Sequence
  Description of Artificial Sequence Synthetic oligonucleotide
aaatcgtgtgtggctctgaaaagagcctttggttaattcctgaggagtgaattgcggccg

SEQ ID NO: 71 - DNA, 60 bp, Artificial Sequence
  Description of Artificial Sequence Synthetic oligonucleotide
tgtgcgccatccacgccaagcgtgtcaccatcatgcccaaggacatccaactggcccgcc

SEQ ID NO: 72 - DNA, 60 bp, Artificial Sequence
  Description of Artificial Sequence Synthetic oligonucleotide
ttcgtttaaggatggagtaaattacagccattttacttgaaatcgtgtgtggctctgaaa

SEQ ID NO: 73 - DNA, 60 bp, Artificial Sequence
  Description of Artificial Sequence Synthetic oligonucleotide
attattcctgaactactctgtgactccaccaggtgaaatagtgagcaagggcgaggagct

SEQ ID NO: 74 - DNA, 60 bp, Artificial Sequence
  Description of Artificial Sequence Synthetic oligonucleotide
gaaggaacctggctttgccctgactttaagcccagatgtttgaggagtgaattgcggccg

SEQ ID NO: 75 - DNA, 60 bp, Artificial Sequence
  Description of Artificial Sequence Synthetic oligonucleotide
ccatgcgcattttagcaccccacaagccttggaattattcctgaactactctgtgactcc

SEQ ID NO: 76 - DNA, 60 bp, Artificial Sequence
  Description of Artificial Sequence Synthetic oligonucleotide
aataaatctttaaaaaaaatatgaaaatatttggaagaaggaaggaacctggctttgccc

SEQ ID NO: 77 - DNA, 60 bp, Artificial Sequence
  Description of Artificial Sequence Synthetic oligonucleotide
cgagggtactaaggccgtcaccaagtacaccagcgctaaggtgagcaagggcgaggagct

SEQ ID NO: 78 - DNA, 60 bp, Artificial Sequence
  Description of Artificial Sequence Synthetic oligonucleotide
ggtggctcttaaaagagccgttagggttgagagtttgcaatgaggagtgaattgcggccg

SEQ ID NO: 79 - DNA, 60 bp, Artificial Sequence
  Description of Artificial Sequence Synthetic oligonucleotide
cctgctgctgcctggggagttggccaagcacgccgtgtccgagggtactaaggccgtcac

SEQ ID NO: 80 - DNA, 60 bp, Artificial Sequence
  Description of Artificial Sequence Synthetic oligonucleotide
aggaggaatacaagcaccagctctttctttgagaacatgggtggctcttaaaagagccgt

SEQ ID NO: 81 - DNA, 60 bp, Artificial Sequence
  Description of Artificial Sequence Synthetic oligonucleotide
catgcacaaactcttcctggagatgctggaggccaaggtggtgagcaagggcgaggagct

SEQ ID NO: 82 - DNA, 60 bp, Artificial Sequence
  Description of Artificial Sequence Synthetic oligonucleotide
cgaggctggtggctgtggaggtctccacttggatcgtgtctgaggagtgaattgcggccg

SEQ ID NO: 83 - DNA, 60 bp, Artificial Sequence
  Description of Artificial Sequence Synthetic oligonucleotide
acacttctacagtgtgaaactgcagggcaaggtgcccatgcacaaactcttcctggagat

SEQ ID NO: 84 - DNA, 60 bp, Artificial Sequence
  Description of Artificial Sequence Synthetic oligonucleotide
ctgggacagctcagagccccgatgcgggtgtgaaaaaagtcgaggctggtggctgtggag

SEQ ID NO: 85 - DNA, 60 bp, Artificial Sequence
  Description of Artificial Sequence Synthetic oligonucleotide
tgttacccatgcggcagaaggcctgggaagactgggacaggtgagcaagggcgaggagct

SEQ ID NO: 86 - DNA, 60 bp, Artificial Sequence
  Description of Artificial Sequence Synthetic oligonucleotide
tcacgtttcacactctttattgaccttcaggaagggccagtgaggagtgaattgcggccg

SEQ ID NO: 87 - DNA, 60 bp, Artificial Sequence
  Description of Artificial Sequence Synthetic oligonucleotide
aggaggtcactgagaaggtcacccacaccatcactgatgctgttacccatgcggcagaag

SEQ ID NO: 88 - DNA, 60 bp, Artificial Sequence
  Description of Artificial Sequence Synthetic oligonucleotide
attccatccaaacagaggtaaactcaggactctgttcacgtttcacactctttattgacc

SEQ ID NO: 89 - DNA, 60 bp, Artificial Sequence
  Description of Artificial Sequence Synthetic oligonucleotide
ctccgtgcgacaggattcttggtgtgctctggccctggccgtgagcaagggcgaggagct

SEQ ID NO: 90 - DNA, 60 bp, Artificial Sequence
  Description of Artificial Sequence Synthetic oligonucleotide
aatatcagacacaagtggtatgaggccttcagagccctcctgaggagtgaattgcggccg

SEQ ID NO: 91 - DNA, 60 bp, Artificial Sequence
  Description of Artificial Sequence Synthetic oligonucleotide
cataggtgtcagtctgtcctcccctgccgaagtcacatcctccgtgcgacaggattcttg

SEQ ID NO: 92 - DNA, 60 bp, Artificial Sequence
  Description of Artificial Sequence Synthetic oligonucleotide
gtctgcatttttgctgccatctggactgctggacaatatcagacacaagtggtatgaggc

SEQ ID NO: 93 - DNA, 60 bp, Artificial Sequence
  Description of Artificial Sequence Synthetic oligonucleotide
caggtcggaccaccttgccttacacatgaagaggcactttgtgagcaagggcgaggagct

SEQ ID NO: 94 - DNA, 60 bp, Artificial Sequence
  Description of Artificial Sequence Synthetic oligonucleotide
aaaaaaaatactgaactctctctcctggcagtgtgggtcatgaggagtgaattgcggccg

SEQ ID NO: 95 - DNA, 60 bp, Artificial Sequence
  Description of Artificial Sequence Synthetic oligonucleotide
accggccctttcagtgccagaagtgtgacagggccttttccaggtcggaccaccttgcct

SEQ ID NO: 96 - DNA, 60 bp, Artificial Sequence
  Description of Artificial Sequence Synthetic oligonucleotide
tcccctcgtgggaagacagtgtgaaaggttagaaaaaaaaatactgaactctctctcctg

SEQ ID NO: 97 - DNA, 60 bp, Artificial Sequence
  Description of Artificial Sequence Synthetic oligonucleotide
agctgacgaagaagccatgacacagatcatccgagtttccgtgagcaagggcgaggagct

SEQ ID NO: 98 - DNA, 60 bp, Artificial Sequence
  Description of Artificial Sequence Synthetic oligonucleotide
ccatttccagaacagtggagaggaccgtgactgatcagcttgaggagtgaattgcggccg

SEQ ID NO: 99 - DNA, 60 bp, Artificial Sequence
  Description of Artificial Sequence Synthetic oligonucleotide
aggactgttgtgctgtctctctctgtaggatccaaaccaagctgacgaagaagccatgac

SEQ ID NO: 100 - DNA, 60 bp, Artificial Sequence
  Description of Artificial Sequence Synthetic oligonucleotide
agtgagactgtcagtgccccactggaagtcagtccatttccagaacagtggagaggaccg

SEQ ID NO: 101 - DNA, 60 bp, Artificial Sequence
  Description of Artificial Sequence Synthetic oligonucleotide
tcagcctgagccagaggctgagaatgaggacaggaccgccgtgagcaagggcgaggagct

SEQ ID NO: 102 - DNA, 60 bp, Artificial Sequence
  Description of Artificial Sequence Synthetic oligonucleotide
gtgttgaagatatatgattcagtcggtctgggtggctcagtgaggagtgaattgcggccg

SEQ ID NO: 103 - DNA, 60 bp, Artificial Sequence
  Description of Artificial Sequence Synthetic oligonucleotide
tgggggaatctgctgctgaagaaggcaccgctaagcggtctcagcctgagccagaggctg

SEQ ID NO: 104 - DNA, 60 bp, Artificial Sequence
  Description of Artificial Sequence Synthetic oligonucleotide
tatgaaaggtgcgtgtgttgaaaggtatgcaggagtgttgaagatatatgattcagtcgg

SEQ ID NO: 105 - DNA, 60 bp, Artificial Sequence
  Description of Artificial Sequence Synthetic oligonucleotide
cggcacggccattaacggcacactgcccctgtcgcacatggtgagcaagggcgaggagct

SEQ ID NO: 106 - DNA, 60 bp, Artificial Sequence
  Description of Artificial Sequence Synthetic oligonucleotide
cctcccaattcccttgtatctctttgaaaatctctccccttgaggagtgaattgcggccg

SEQ ID NO: 107 - DNA, 60 bp, Artificial Sequence
  Description of Artificial Sequence Synthetic oligonucleotide
gactgcacatggcccagcactaccagagcggcccggtgcccggcacggccattaacggca

SEQ ID NO: 108 - DNA, 60 bp, Artificial Sequence
  Description of Artificial Sequence Synthetic oligonucleotide
attatcagatttttcctactctcctctttttgcacccctcccaattcccttgtatctctt

SEQ ID NO: 109 - DNA, 60 bp, Artificial Sequence
  Description of Artificial Sequence Synthetic oligonucleotide
ttgtcttttcctccaacgtttttacgagccgtcgaagatggctagcaaaggagaagaact

SEQ ID NO: 110 - DNA, 60 bp, Artificial Sequence
  Description of Artificial Sequence Synthetic oligonucleotide
aagtggctatctccagcaaccaaaaagtcaaggttatcgcgattttaccacatttgtaga

SEQ ID NO: 111 - DNA, 60 bp, Artificial Sequence
  Description of Artificial Sequence Synthetic oligonucleotide
tggctttatgaactaaagccatctgctaatattgtgtttcttgtcttttcctccaacgtt

SEQ ID NO: 112 - DNA, 60 bp, Artificial Sequence
  Description of Artificial Sequence Synthetic oligonucleotide
gcaagacaaaaatcagagcgtcatagaacgtgattttccgaagtggctatctccagcaac

SEQ ID NO: 113 - DNA, 60 bp, Artificial Sequence
  Description of Artificial Sequence Synthetic oligonucleotide
aggaagcagctaagacaacatgaatgaacaaaaaatgaatgtgagcaagggcgaggagct

SEQ ID NO: 114 - DNA, 60 bp, Artificial Sequence
  Description of Artificial Sequence Synthetic oligonucleotide
gggctcttccgcccggccctttctggccacttgtcttcgcgattttaccacatttgtaga

SEQ ID NO: 115 - DNA, 60 bp, Artificial Sequence
  Description of Artificial Sequence Synthetic oligonucleotide
gatcagtgccccctggaagtgagtcataggcattgttcaagaaggaagcagctaagacaa

SEQ ID NO: 116 - DNA, 60 bp, Artificial Sequence
  Description of Artificial Sequence Synthetic oligonucleotide
actggccttgcctcgtcttgctttagggtcagtctgtcgagggctcttccgcccggccct

SEQ ID NO: 117 - DNA, 21 bp, Artificial Sequence
  Description of Artificial Sequence Synthetic oligonucleotide
ccttgtgggtctgtttgagga

SEQ ID NO: 118 - DNA, 20 bp, Artificial Sequence
  Description of Artificial Sequence Synthetic oligonucleotide
gtctttgctcagggcggact

SEQ ID NO: 119 - DNA, 90 bp, Artificial Sequence
  Description of Artificial Sequence Synthetic oligonucleotide
tgttttaaaatggactatcatatgcttaccgtaacttgaaagtatttcgatttcttggctttatatatcttgtggaaaggacgaaacacc

SEQ ID NO: 120 - DNA, 60 bp, Artificial Sequence; identical to SEQ ID NO: 47
  Description of Artificial Sequence Synthetic oligonucleotide

SEQ ID NO: 121 - DNA, 90 bp, Artificial Sequence
  Description of Artificial Sequence Synthetic oligonucleotide
gtacaaaatacgtgacgtagaaagtaataatttcttgggtagtttgcagttttaaaattatgttttaaaatggactatcatatgcttacc

SEQ ID NO: 122 - DNA, 90 bp, Artificial Sequence; identical to SEQ ID NO: 49
  Description of Artificial Sequence Synthetic oligonucleotide

SEQ ID NO: 123 - DNA, 90 bp, Artificial Sequence
  Description of Artificial Sequence Synthetic oligonucleotide
cgatacaaggctgttagagagataattagaattaatttgactgtaaacacaaagatattagtacaaaatacgtgacgtagaaagtaataa

SEQ ID NO: 124 - DNA, 60 bp, Artificial Sequence
  Description of Artificial Sequence Synthetic oligonucleotide
tcaatgtatcttatcatgtctgctcgattttaacttgctatttctagctctaaaacaaaa

SEQ ID NO: 125 - DNA, 60 bp, Artificial Sequence
  Description of Artificial Sequence Synthetic oligonucleotide
ggcctatttcccatgattccttcatatttgcatatacgatacaaggctgttagagagata

SEQ ID NO: 126 - DNA, 27 bp, Artificial Sequence
  Description of Artificial Sequence Synthetic oligonucleotide
tcaatgtatcttatcatgtctgctcga

SEQ ID NO: 127 - DNA, 415 bp, Artificial Sequence
  Description of Artificial Sequence Synthetic polynucleotide
ggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggcgtctgggtggctcttggtttaagagctatgctggaaacagcatagcaagtttaaataaggctagtccgttatcaacttgaaaaagtggcaccgagtcggtgcttttttgttttagagctagaaatagcaagttaaaatcgagcagacatgataagatacattga

SEQ ID NO: 128 - DNA, 503 bp, Artificial Sequence
  Description of Artificial Sequence Synthetic polynucleotide
cgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagctgaattttgttttccttcag

SEQ ID NO: 129 - DNA, 60 bp, Artificial Sequence
  Description of Artificial Sequence Synthetic oligonucleotide
ggaaaggacgaaacaccgacagcatccccagggagggcgtttaagagctatgctggaaac

SEQ ID NO: 130 - DNA, 739 bp, Artificial Sequence
  Description of Artificial Sequence Synthetic polynucleotide
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcaattcactcctca

SEQ ID NO: 131 - DNA, 60 bp, Artificial Sequence
  Description of Artificial Sequence Synthetic oligonucleotide
tgttcccgtcactgctctgggctctcccatgcattcaaacgtgagcaagggcgaggagct

SEQ ID NO: 132 - DNA, 60 bp, Artificial Sequence
  Description of Artificial Sequence Synthetic oligonucleotide
ccaggttctcttgtctacctcccttgccttggctcacagctgaggagtgaattgcggccg

SEQ ID NO: 133 - DNA, 60 bp, Artificial Sequence
  Description of Artificial Sequence Synthetic oligonucleotide
tctactcagtcccttttcctgagggcgaggcctttccctctgttcccgtcactgctctgg

SEQ ID NO: 134 - DNA, 60 bp, Artificial Sequence
  Description of Artificial Sequence Synthetic oligonucleotide
taatccctcctcagtaaaagaatttaaccccaaagctccaggttctcttgtctacctccc

SEQ ID NO: 135 - DNA, 19 bp, Artificial Sequence
  Description of Artificial Sequence Synthetic oligonucleotide
gtccagctcgaccaggatg

SEQ ID NO: 136 - DNA, 20 bp, Artificial Sequence
  Description of Artificial Sequence Synthetic oligonucleotide
ggatcactctcggcatggac

SEQ ID NO: 137 - DNA, 20 bp, Artificial Sequence
  Description of Artificial Sequence Synthetic oligonucleotide
tgtatcctttcctctgcccc

SEQ ID NO: 138 - DNA, 20 bp, Artificial Sequence
  Description of Artificial Sequence Synthetic oligonucleotide
gagcttctttccccatccca

SEQ ID NO: 139 - DNA, 896 bp, Artificial Sequence
  Description of Artificial Sequence Synthetic polynucleotide
tctactcagtcccttttcctgagggcgaggcctttccctctgttcccgtcactgctctgggctctcccatgcattcaaacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcaattcactcctcagctgtgagccaaggcaagggaggtagacaagagaacctggagctttggggttaaattcttttactgaggagggatta

SEQ ID NO: 140 - DNA, 21 bp, Artificial Sequence
  Description of Artificial Sequence Synthetic oligonucleotide
tttagctcgccgtccagctcg

SEQ ID NO: 141 - RNA, 21 nt, Artificial Sequence
  Description of Artificial Sequence Synthetic oligonucleotide
uuuacgucgccguccagcucg

SEQ ID NO: 142 - DNA, 24 bp, Artificial Sequence
  Description of Artificial Sequence Synthetic oligonucleotide
  modified_base (3)..(3): a, c, t, g, unknown or other
ggncgagctggacggcgacgtaaa

SEQ ID NO: 143 - DNA, 179 bp, Unknown
  Description of Unknown Wildtype GFP sequence
  modified_base (30)..(149): a, c, t, g, unknown or other
tggtcgagctggacggcgacgtaaacggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacctacggcgtgcagtgcttcagccgctac

SEQ ID NO: 144 - DNA, 47 bp, Artificial Sequence
  Description of Artificial Sequence Synthetic oligonucleotide
agctggacggcgacgtgcgtgcagtgcttcagccgctaccccgacca

SEQ ID NO: 145 - DNA, 31 bp, Artificial Sequence
  Description of Artificial Sequence Synthetic oligonucleotide
acaccgctctgtgactagtcacagaggttta

SEQ ID NO: 146 - RNA, 21 nt, Artificial Sequence
  Description of Artificial Sequence Synthetic oligonucleotide
gcucugugacuagucacagag

(19) United States
(12) Patent Application Publication
(10) Pub. No.: US 2019/0002920 A1
(43) Pub. Date: Jan. 3, 2019

(54) METHODS AND KITS FOR CLONING-FREE GENOME EDITING

(71) Applicant: THE BRIGHAM AND WOMEN'S HOSPITAL, INC., Boston, MA (US)

(72) Inventors: Richard Sherwood, Boston, MA (US); Mandana Arbab, Amsterdam (NL)

(73) Assignee: THE BRIGHAM AND WOMEN'S HOSPITAL, INC., Boston, MA (US)

(21) Appl. No.: 15/569,232

(22) PCT Filed: Apr. 28, 2016
(86) PCT No.: PCT/US16/29695

§ 371 Date: Oct. 25, 2017

Related U.S. Application Data
(60) Provisional application No. 62/154,790, filed on Apr. 30, 2015.

(51) Int. Cl. C12N 15/90; C12N 15/11; C12N 9/22; C12N 15/10

(52) U.S. Cl. CPC C12N 15/907; C12N 15/11; C12N 2800/80; C12N 15/102; C12N 2310/20; C12N 9/22

(57) ABSTRACT

The methods and compositions provided herein improve upon the methods presently used for targeted genomic modification, in part, by removing the requirement for sub-cloning of a sequence complementary to a site selected for genomic modification. The methods and compositions provided herein can be used in place of a standard CRISPR/Cas system to provide simple, fast, and inexpensive targeted modification of a genome. The methods and compositions provided herein can also be used in high-throughput genome editing applications.

### CROSS-REFERENCE TO RELATED APPLICATION

This Application claims benefit under 35 U.S.C. § 119(e) of the U.S. Provisional Application No. 62/154,790 filed Apr. 30, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 28, 2016, is named 043214-084481-PCT_SL.txt and is 42,116 bytes in size.

### FIELD OF THE INVENTION

The present invention relates to methods and compositions for targeted modification of a genome sequence.

### BACKGROUND

The CRISPR (clustered regularly interspaced short palindromic repeats)/Cas9 (CRISPR-associated protein-9) system has emerged as an efficient tool to mutate, delete and insert genomic DNA sequences in a site-specific manner^{1,2}. In CRISPR-mediated genome editing, Cas9 protein is directed to cleave DNA by an associated single guide RNA (sgRNA) hairpin structure that can be designed to target almost any genomic site of interest. Site specific mutagenesis and targeted transgenesis are key applications for studying development and disease, and as such, the ability to easily edit any genomic locus is revolutionizing stem cell research.

### SUMMARY

The methods and compositions provided herein improve upon the methods presently used for targeted genomic modification, in part, by removing the requirement for sub-cloning of a sequence complementary to a site selected for genomic modification. The methods and compositions provided herein can be used in place of a standard CRISPR/Cas system to provide simple, fast, and inexpensive targeted modification of a genome. The methods and compositions provided herein can also be used in high-throughput genome editing applications.

Provided herein in one aspect is a method of generating a plasmid intracellularly for targeted modification of a genomic sequence, the method comprising introducing to the cell: (a) an expression construct encoding an RNA-guided endonuclease; and (b) a plasmid encoding a sequence directing the transcription of a self-targeting RNA guide sequence, comprising a self-targeting sequence, wherein the self-targeting RNA forms a complex with the RNA-guided endonuclease to initiate cleavage of a self-targeted sequence in the plasmid sequence encoding the self-targeting RNA guide sequence, such that transcription of the self-targeting RNA in the presence of the RNA-guided endonuclease permits the formation of a complex with the RNA guided endonuclease that directs the cleavage of the plasmid within the self-targeted sequence; and (c) a repair template (e.g., a single or double-stranded DNA) comprising a genomic targeting sequence flanked by first and second homology arms homologous, respectively, to sequences that flank said self-targeting sequence in the plasmid, the genomic targeting sequence sufficient to direct cleavage by an associated RNA-guided nuclease to a genomic target sequence, wherein, upon introduction of the expression construct encoding an RNA-guided endonuclease, the plasmid and the repair template (e.g., a single or double-stranded DNA) to the cell, the plasmid is cleaved in the self-targeted sequence and the repair template comprising the genomic targeting sequence directs the homologous replacement of the self-targeted sequence with the genomic targeting sequence, whereby the cleavage-guiding specificity of the self-targeted guide RNA is modified to the genomic target sequence.

In one embodiment of this aspect and all other aspects described herein, the expressed RNA-guided endonuclease forms a complex with the modified guide RNA expressed from the plasmid and the complex with modified guide RNA effects targeted modification of the genomic target sequence.

In another embodiment of this aspect and all other aspects described herein, the expression construct is a plasmid.

In another embodiment of this aspect and all other aspects described herein, the endonuclease is a Cas endonuclease.

In another embodiment of this aspect and all other aspects described herein, the Cas endonuclease is Cas9.

In another embodiment of this aspect and all other aspects described herein, the self-targeting sequence comprises a palindromic sequence.

In another embodiment of this aspect and all other aspects described herein, the RNA-guided endonuclease introduces a double-stranded break in the genomic target sequence.

In another embodiment of this aspect and all other aspects described herein, the method does not require cloning of a sequence into a cloning vector.

In another embodiment of this aspect and all other aspects described herein, the method further comprises providing a linear single or double-stranded DNA repair template for homologous recombination-mediated repair at the selected genomic target sequence.

In another embodiment of this aspect and all other aspects described herein, the process of homologous recombination inactivates the target sequence.

In another embodiment of this aspect and all other aspects described herein, the repair template comprises an engineered DNA sequence flanked by first and second homology arms homologous, respectively, to sequences that flank the selected genomic targeting sequence.

In another embodiment of this aspect and all other aspects described herein, the engineered DNA sequence comprises a sequence encoding one or more nucleotide mutation(s), one or more inserted nucleotide(s), or one or more deleted nucleotide(s).

In another embodiment of this aspect and all other aspects described herein, each of the self-targeted guide RNA sequence and the guide RNA expressed from the modified plasmid comprises at least one hairpin.

In another embodiment of this aspect and all other aspects described herein, each of the self-targeted guide RNA sequence and the guide RNA expressed from the modified plasmid comprises a crRNA and/or a tracrRNA sequence to permit association of the guide RNA with the RNA-guided endonuclease.

In another embodiment of this aspect and all other aspects described herein, the crRNA and/or tracrRNA sequence is codon optimized for the organism comprising the selected genomic target sequence.

In another embodiment of this aspect and all other aspects described herein, the crRNA and tracrRNA sequence comprise a fusion sequence.

In another embodiment of this aspect and all other aspects described herein, the expression construct and the plasmid are introduced to the cell by electroporation, transfection, or viral delivery.

In another embodiment of this aspect and all other aspects described herein, the expression construct or the plasmid further comprises a sequence encoding a reporter molecule.

In another embodiment of this aspect and all other aspects described herein, the reporter molecule is GFP.

Another aspect provided herein relates to a composition comprising a nucleic acid vector encoding a sequence directing the transcription of a self-targeting RNA guide molecule for an RNA-guided endonuclease, the sequence comprising a self-targeting sequence, wherein when contacted with the RNA guided endonuclease, self-targeting RNA guide molecule transcribed from the vector forms a complex with the RNA-guided endonuclease, and wherein the complex cleaves the plasmid in the sequence encoding the self-targeting RNA guide molecule, such that transcription of the self-targeting RNA in the presence of the RNA-guided endonuclease results in cleavage of the nucleic acid vector in the sequence encoding the self-targeting RNA guide molecule.

In one embodiment of this aspect and all other aspects described herein, the nucleic acid vector further encodes an RNA-guided endonuclease.

In another embodiment of this aspect and all other aspects described herein, the endonuclease is a Cas endonuclease.

In another embodiment of this aspect and all other aspects described herein, the Cas endonuclease is Cas9.

In another embodiment of this aspect and all other aspects described herein, the self-targeting sequence comprises a palindromic sequence.

In another embodiment of this aspect and all other aspects described herein, the RNA-guided endonuclease introduces a double-stranded break in the targeted sequence.

In another embodiment of this aspect and all other aspects described herein, the self-targeting RNA guide molecule comprises at least one hairpin.

In another embodiment of this aspect and all other aspects described herein, the self-targeting RNA guide molecule comprises a crRNA and/or a tracrRNA sequence to permit association of the guide RNA with the RNA-guided endonuclease.

In another embodiment of this aspect and all other aspects described herein, the crRNA and/or tracrRNA sequence is codon optimized for an organism in which targeted modification of the genome is desired.

In another embodiment of this aspect and all other aspects described herein, the crRNA and tracrRNA sequence comprise a fusion sequence.

Also provided herein, in another aspect, is a composition comprising a vector composition as described herein and a linear repair template comprising a genomic targeting sequence, flanked by first and second homology arms homologous, respectively, to sequences that flank the self-targeting sequence in the vector.

In one embodiment of this aspect and all other aspects described herein, the linear repair template is single-stranded DNA. In another embodiment of this aspect and all other aspects described herein, the linear repair template is double-stranded DNA.

Another aspect provided herein relates to a kit comprising any one of the compositions described herein and instructions therefor

In another embodiment of this aspect and all other aspects described herein, the kit further comprises an expression construct encoding an RNA-guided endonuclease.

Another aspect described herein relates to a cell comprising a composition as described herein, for example, a composition as described in any one of claims 20-30 as filed.

In one embodiment of this aspect and all other aspects described herein, the cell is a mammalian cell, a plant cell, an insect cell, or a cell of a pathogen or pest.

In another embodiment of this aspect and all other aspects described herein, cell is a human cell.

In another embodiment of this aspect and all other aspects provided herein, the cell is a cancer cell.

### BRIEF DESCRIPTION OF THE FIGURES

FIGS. 1A-1G Linear-CRISPR enables efficient cloning-free knock-in transgenesis. FIG. 1A shows a schematic of Linear-CRISPR. FIG. 1B are histograms showing flow cytometric Histone H3.1 GFP fluorescence (x-axis) after electroporation with Cas9 and sgGFP linear product or plasmid. FIG. 1C is fluorescence microscopy data showing loss of Histone H3.1-GFP fluorescence in mESCs after targeting with Cas9 and sgGFP linear product or plasmid. FIG. 1D is a flow cytometric analysis showing efficient generation of Histone H3.1-GFP knock-in cells (y-axis) after Linear-CRISPR and plasmid-based CRISPR using a PCR-amplified GFP fragment with 80 bp Histone H3.1 homology arms. FIG. 1E shows fluorescence microscopy of Histone H3.1-GFP mESCs generated through Linear-CRISPR-based knock in. FIG. 1F is data from a flow cytometric analysis that shows efficient generation of Histone H2BJ-GFP knock-in cells (y-axis) after Linear-CRISPR in HUES2 human embryonic stem cells. FIG. 1G shows fluorescence microscopy of HUES2 Histone H2BJ-GFP human embryonic stem cells generated through Linear-CRISPR and plasmid-based CRISPR knock-in.

FIGS. 2A-2H Simplified, efficient genome editing using Self-Cloning CRISPR. FIG. 2A is a schematic of the Self-Cloning CRISPR process that occurs inside target cells. FIG. 2B are histograms showing flow cytometric Histone H3.1-GFP fluorescence (x-axis) after electroporation with Cas9, sgPal plasmid, and homology fragment(s). FIG. 2C is fluorescence microscopy showing loss of Histone H3.1-GFP fluorescence in mESCs after targeting with Cas9, sgPal plasmid, and sgGFP homology fragment. FIG. 2D shows a multiplexed mutation of GFP (x-axis) and dsRed (y-axis) through co-introduction of Cas9, sgPal plasmid, and sgGFP and sgDsRed homology fragments.

FIG. 2E shows data from a flow cytometric analysis indicating efficient generation of Histone H3.1-GFP knock-in mES cells (y-axis) after scCRISPR using a PCR-amplified GFP fragment with 80 bp Histone H3.1 homology arms. FIG. 2F is fluorescence microscopy of Histone H3.1-GFP mESCs generated through scCRISPR-based knock-in. FIG. 2G is a flow cytometric analysis that shows efficient generation of Histone H2BJ-GFP knock-in hES cells (y-axis) after scCRISPR using a PCR-amplified GFP fragment with 80 bp Histone H3.1 homology arms. FIG. 2H shows fluorescence microscopy of Histone H3.1-GFP hESCs generated through scCRISPR based knock-in.

FIG. 3 Comparison of time, cost, and efficiency among different methods of CRISPR mutation and homologous recombination.

FIGS. 4A-4C scCRISPR and Linear-CRISPR show efficient plasmid-free homologous recombination to produce site-specific GFP knock-in mESCs. FIG. 4A is a flow cytometric analysis indicating efficient generation of Nanog-GFP knock-in mES cells (y-axis) after scCRISPR, Linear-CRISPR, and plasmid-based CRISPR using a PCR-amplified GFP fragment with 80 bp Nanog homology arms. FIG. 4B shows a genomic DNA PCR analysis using a forward primer in the Histone H3.1 coding region and a reverse primer in the GFP coding region that will produce a 166 band only if GFP is inserted into the Histone H3.1 locus. scCRISPR and Linear-CRISPR-based knock-in using PCR-amplified GFP fragments with 80 bp Histone H3.1 homology arms show robust bands indicating successful knock-in. FIG. 4C is a flow cytometric analysis of HEK293T that shows efficient generation of H2BJ-GFP knock-in cells (y-axis) after, Linear-CRISPR using a PCR-amplified GFP fragment with 80 bp Nanog homology arms.

FIGS. 5A-5B Generation of nine additional clonal mESC GFP knock-in lines. FIG. 5A is a flow cytometric analysis that shows efficient generation of GFP knock-in at four loci in mESC (y-axis) after Linear-CRISPR using a PCR-amplified GFP fragment with 80 bp homology arms. Nfya, Rpp25, and Sox2 lines are C-terminal GFP fusion proteins and Zfp42 is a GFP replacement. FIG. 5B is a flow cytometric analysis of nine clonal mESC knock-in lines generated using Linear-CRISPR. All are C-terminal GFP fusion cell lines except Tdgf1 and Zfp42, which are GFP replacements. All lines have clonal knock-in in every cell, but GFP fluorescence intensities vary based on the native gene expression levels. Bulk measurements of GFP fluorescence were only performed for the four loci in FIG. 5A.

FIGS. 6A-6B scCRISPR induces indels with a single homology fragment and deletion with two homology fragments. FIG. 6A is a Sanger sequencing analysis of genomic DNA from two Histone H3.1-GFP-clones produced through scCRISPR with sgPal1 and sgGFP1, showing short deletions surrounding the expected CRISPR cut site. FIG. 6B is a Sanger sequencing analysis of a gel-isolated deletion band from bulk genomic DNA of Histone H3.1-GFPb cells after multiplexed scCRISPR with sgPal1, sgGFP2, and sgGFP3. This band shows a 134 bp deletion with junctions at the predicted CRISPR cut sites.

FIG. 7 scCRISPR efficiency is dependent on sgRNA plasmid self-cleavage and homology fragment length. Histograms showing flow cytometric Histone H3.1-GFP fluorescence (x-axis) after electroporation with Cas9, sgRNA plasmid, and homology fragment. Palindromic sgRNA plasmids (sgPal2-4) all exhibit substantially more GFP loss than a non-palindromic sgRNA plasmid (sgnonPal) indicating that self cleavage is an important factor in scCRISPR efficiency. A homology fragment with 30 bp of homology shows substantially less GFP loss than the standard 90 bp (FIG. 3), indicating that homology arm length is also important in scCRISPR efficiency. Without wishing to be bound by theory, these results indicate that scCRISPR functions through homologous recombination.

FIGS. 8A-8B scCRISPR reliably induces mutation across multiple sgRNAs and when multiplexed. Histograms showing flow cytometric Histone H3.1-GFP fluorescence (x-axis, FIG. 8A) or DsRed fluorescence (x-axis, FIG. 8B) after electroporation with Cas9, sgRNA plasmid, and homology fragment. In these plots, sgPal is combined with homology fragments targeting two additional sites within the GFP gene (sgGFP1 and sgGFP2) as well as two locations within the dsRed gene (sgdsRed1 and sgDsRed2), producing >55% loss of fluorescence in all cases. Additionally, multiplexing sgDsRed1 and sgDsRed2 increases the fraction of cells with loss of dsRed fluorescence (FIG. 8B), while multiplexing sgGFP2 and sgDsRed1 in single positice Histone H3.1-GFP mES cells only minimally decreases the fraction of cells with loss of GFP fluorescence (FIG. 8A), indicating that scCRISPR can lead to efficient and specific multiplexed mutation.

FIG. 9 Palindromic sgRNA targeting sequence in the context of plasmid DNA

FIGS. 10A-10C Self-Cloning CRISPR Gene Editing. FIG. 10A is a schematic of the scCRISPR process within target cells. The self-cleaving sgPal plasmid recombines with the short PCR-amplified sgRNA template to form a new site-specific sgRNA plasmid, to facilitate genome editing. FIG. 10B is a histogram of flow cytometric GFP fluorescence (x-axis) of Hist1h3a mouse ESCs after electroporation with sgPal plasmid alone (left), or together with sgGFP homology fragment (right). FIG. 10C is a flow cytometric plot of efficient generation of Pou5f1-GFP knock-in mouse ESCs (y-axis) using PCR-amplified GFP fragment.

### DETAILED DESCRIPTION

Provided herein are cloning-free methods for targeted modification of a selected genomic sequence that can be completed in a very short time frame, for example, same-day genomic modification. Among the aspects described are methods involving introducing a self-cleaving plasmid that encodes a guide RNA and a short repair template sequence encoding a desired locus-specific guide RNA into cells, permitting them to produce a locus-specific guide RNA plasmid through homologous recombination intracellularly. This approach obviates the need, for example, to separately clone a guide RNA construct targeting the desired locus. This and other aspects are described in further detail herein below.

### Definitions

As used herein, the term “generating a plasmid intracellularly” refers to a method where a plasmid sequence comprising a desired genomic targeting sequence is obtained within a cell through the use of the cell's machinery (e.g., via homologous recombination) and does not require the use of in vitro sub-cloning methods involving restriction enzymes and/or ligation enzymes.

The phrase “targeted modification of a genomic sequence” is used herein to refer to the modification of a genomic sequence at a unique target site in the genome; that is, the modification occurs at a single site and produces little to no off-target effects at other sites in the genome. In one embodiment, the term ‘targeted modification of a genomic sequence’ means that the modification of the genomic sequence occurs only at the unique target sequence and does not target any other sites in the host genome.

As used herein, the term “self-targeting RNA guide sequence” refers to an RNA sequence expressed from a plasmid and comprising (i) a sequence that permits association with an RNA-guided endonuclease enzyme and (ii) a targeting sequence that recognizes and binds the nucleic acid sequence from which the self-targeting RNA guide sequence was expressed (i.e., “the self-targeted sequence” or “targeted sequence”), and directs cleavage of the targeted sequence by the RNA-guided endonuclease.

Cleavage of the self-targeted site permits recombination using a double-stranded DNA carrying a genomic targeting sequence such that the self-targeting sequence is replaced with the genomic targeting sequence, without the need to sub-clone the genomic sequence into the guide RNA plasmid vector. In one embodiment, the self-targeting sequence comprises a palindromic sequence.

As used herein, the term “inactivates the target sequence” is used to refer to the homologous replacement of the nucleic acid sequence encoding the self-targeting guide RNA sequence with a sequence encoding a genomic targeting guide RNA sequence, such that expression of the genomic targeting guide RNA occurs from the plasmid while the self-targeting guide RNA is no longer expressed.

As used herein, the term “homologous replacement” when used to refer to the replacement of the self-targeted sequence with the genomic targeting sequence, refers to a homologous recombination or “crossover” event triggered by a double-strand break generated by the RNA-guided endonuclease; the crossover event causes a replacement of the self-targeted sequence on the plasmid with the genomic targeting sequence (introduced to the cell via the double-stranded DNA sequence), such that the genomic targeting sequence is introduced into the plasmid and interrupts the self-targeted sequence. The homologous recombination event essentially replaces an in vitro sub-cloning method (i.e., ligation of a desired double-stranded DNA sequence into a specific site in a linearized and subsequently recircularized plasmid), by using the cellular machinery to generate a plasmid comprising a sequence encoding a guide RNA sequence which itself comprises a genomic targeting sequence.

The terms “repair template” or “linear repair template” are used interchangeably herein and refer to a single- or double-stranded DNA template for effecting homologous recombination to introduce a desired sequence at the site of the double-stranded break induced by Cas9 cleavage. Such repair templates comprise a desired nucleic acid sequence flanked by first and second homology arms homologous, respectively, to sequences that flank the targeted sequence at the site of Cas9 cleavage.

As used herein, the term “codon optimized” or “codon optimization” refers to the process of modifying a nucleic acid sequence for enhanced expression in the cells of the vertebrate of interest, e.g., mouse, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

The term “promoter” refers to a nucleic acid sequence that is typically positioned upstream of a gene and that recruits transcriptional machinery, such as the RNA polymerase and associated factors, that, in turn, initiates transcription of the gene.

The term “operably linked” refers to the joining of distinct DNA molecules, or DNA sequences, to produce a functional transcriptional unit.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, “a” or “an” means at least one, unless clearly indicated otherwise. As used herein, to “prevent” or “protect against” a condition or disease means to hinder, reduce or delay the onset or progression of the condition or disease.

The term “statistically significant” or “significantly” refers to statistical significance and generally means two standard deviations (2SD) or more above or below normal or a reference. The term refers to statistical evidence that there is a difference. It is defined as the probability of making a decision to reject the null hypothesis when the null hypothesis is actually true. The decision is often made using the p-value.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), and Current Protocols in Protein Science (CPPS), John E. Coligan (ed.), John Wiley and Sons, Inc., 2005 (ISBN 0471142735), the contents of which are all incorporated by reference herein in their entireties.

### RNA-Guided Endonucleases

As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to the selected target DNA sequence. In one embodiment, the RNA-guided endonuclease is a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In one embodiment, the Cas protein is Cas9.

Typically, the RNA-guided endonuclease comprises DNA cleavage activity, such as the double strand breaks initiated by Cas9. In some embodiments, the RNA-guided endonuclease is Cas9, for example, Cas9 from S. pyogenes or S. pneumoniae. In some embodiments, the RNA-guided endonuclease comprises nickase activity. In some embodiments, the RNA-guided endonuclease directs cleavage of one or both strands at the location of a target sequence, such as within the target sequence and/or within the complement of the target sequence. In some embodiments, the RNA-guided endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, an expression construct or vector encodes an RNA-guided endonuclease that is mutated with respect to a corresponding wild-type enzyme such that the mutated endonuclease lacks the ability to cleave one strand of a target polynucleotide containing a target sequence. For example, an aspartate-to-alanine substitution (D 10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase can be used in combination with guide sequence(s), e.g., two guide sequences, which target respectively sense and antisense strands of the DNA target. This combination allows both strands to be nicked and used to induce non-homologous end joining (NHEJ) repair.

In some embodiments, the nucleic acid sequence encoding the RNA-guided endonuclease is codon optimized for expression in particular cells, such as eukaryotic cells. The eukaryotic cells can be derived from a particular organism, such as a mammal. Non-limiting examples of mammals can include human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments, the RNA-guided endonuclease is part of a fusion protein comprising one or more heterologous protein domains (e.g. about or more than about 1, 2, 3, 4, 5, 6, 7, 8, 9, 10, or more domains in addition to the endonuclease). An RNA-guided endonuclease fusion protein can comprise any additional protein sequence, and optionally a linker sequence between any two domains.

Examples of protein domains that can be fused to an RNA-guided endonuclease include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). An RNA-guided endonuclease can be fused to a gene sequence encoding a protein or a fragment of a protein that binds DNA molecules or binds to other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. In some embodiments, a tagged endonuclease is used to identify the location of a target sequence.

### Nuclear Localization Signals

In some embodiments, the expression vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 1.

TABLE 1
Nuclear Localization Signals

| | | SEQ ID |
|---|---|---|
| SOURCE | SEQUENCE | NO. |
| SV40 virus large T- | PKKKRKV | 1 |
| antigen | | |
| nucleoplasmin | KRPAATKKAGQAKKKK | 2 |
| c-myc | PAAKRVKLD | 3 |
| | RQRRNELKRSP | 4 |
| hRNPA1 M9 | NQSSNFGPMKGGNFGGRSSG | 5 |
| | PYGGGGQYFAKPRNQGGY | |
| IBB domain from | RMRIZFKNKGKDTAELRRRRV | 6 |
| importin-alpha | EVSVELRKAKKDEQILKRRNV | |
| myoma T protein | VSRKRPRP | 7 |
| | PPKKARED | 8 |
| human p53 | PQPKKKPL | 9 |
| mouse c-abl IV | SALIKKKKKMAP | 10 |
| influenza virus NS1 | DRLRR | 11 |
| | PKQKKRK | 12 |
| Hepatitis virus delta | RKLKKKIKKL | 13 |
| antigen | | |
| mouse Mx1 protein | REKKKFLKRR | 14 |
| human poly(ADP- | KRKGDEVDGVDEVAKKKSKK | 15 |
| ribose) polymerase | | |
| steroid hormone | RKCLQAGMNLEARKTKK | 16 |
| receptors (human) | | |
| glucocorticoid | | |

Guide RNA (gRNA) Sequence(s)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, the gRNA sequence comprises a targeting sequence that directs the gRNA sequence to a desired site in the genome, fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq. In some embodiments, a guide sequence is 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide RNA sequence comprises a palindromic sequence, for example, the self-targeting sequence comprises a palindrome. The targeting sequence of the guide RNA is typically 19-21 basepairs long and directly precedes the hairpin that binds the entire guide RNA (targeting sequence+hairpin) to Cas9. Where a palindromic sequence is employed as the self-targeting sequence of the guide RNA, the inverted repeat element can be e.g., 9, 10, 11, 12, or more nucleotides in length. Where the targeting sequence of the guide RNA is most often 19-21 bp, a palindromic inverted repeat element of 9 or 10 nucleotides provides a targeting sequence of desirable length. The Cas9-guide RNA hairpin complex can then recognize and cut any DNA sequence that matches the 19-21 basepair sequence and is followed by a “PAM” sequence e.g., NGG.

The ability of a guide sequence to direct sequence-specific binding of an RNA-guided endonuclease complex to a target sequence can be assessed by any suitable assay. For example, the components of an RNA-guided endonuclease system sufficient to form an RNA-guided endonuclease complex, including the guide sequence to be tested, can be provided to a host cell having the corresponding target sequence, such as by transfection with vectors encoding the components of the RNA-guided endonuclease sequence, followed by an assessment of preferential cleavage within the target sequence, such as by Surveyor assay (Transgenomic™, New Haven, Conn.). Similarly, cleavage of a target polynucleotide sequence can be evaluated in a test tube by providing the target sequence, components of an RNA-guided endonuclease complex, including the guide sequence to be tested and a control guide sequence different from the test guide sequence, and comparing binding or rate of cleavage at the target sequence between the test and control guide sequence reactions. One of ordinary skill in the art will appreciate that other assays can also be used to test gRNA sequences.

A guide sequence can be selected to target any target sequence. In some embodiments, the target sequence is a sequence within a genome of a cell. In some embodiments, the target sequence is the sequence encoding a first guide RNA in a self-cloning plasmid, as described herein. Typically, the target sequence in the genome will include a protospacer adjacent (PAM) sequence for binding of the RNA-guided endonuclease. It will be appreciated by one of skill in the art that the PAM sequence and the RNA-guided endonuclease should be selected from the same (bacterial) species to permit proper association of the endonuclease with the targeting sequence. To prevent degradation of the guide RNA, the sequence of the guide RNA should not contain the PAM sequence. In some embodiments, the length of the targeting sequence in the guide RNA is 12 nucleotides; in other embodiments, the length of the targeting sequence in the guide RNA is 13, 14, 15, 16, 17, 18, 19, 20, 21, 22, 23, 24, 25, 26, 27, 28, 29, 30, 35 or 40 nucleotides. The guide RNA can be complementary to either strand of the targeted DNA sequence. In some embodiments, when modifying the genome to include an insertion or deletion, the gRNA can be targeted closer to the N-terminus of a protein coding region.

It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10): 1473-1475 (2014); Aach et al. “CasFinder: Flexible algorithm for identifying specific Cas9 targets in genomes” BioRxiv (2014), among others).

For the S. pyogenes Cas9, a unique target sequence in a genome can include a Cas9 target site of the form MMMMMMMMNNNNNNNNNNNXGG where NNNNNNNNNNNNXGG (N is A, G, T, or C; and X can be any nucleotide) has a single occurrence in the genome. A unique target sequence in a genome can include an S. pyogenes Cas9 target site of the form MMMMMMMMMNNNNNNNNNNNXGG where NNNNNNNNNNNXGG (N is A, G, T, or C; and X can be any nucleotide) has a single occurrence in the genome. For the S. thermophilus CRISPR1 Cas9, a unique target sequence in a genome can include a Cas9 target site of the form MMMMMMMMNNNNNNNNNNNNXXAGAAW where NNNNNNNNNNNNXXAGAAW (N is A, G, T, or C; X can be any nucleotide; and W is A or T) has a single occurrence in the genome. A unique target sequence in a genome can include an S. thermophilus CRISPR 1 Cas9 target site of the form MMMMMMMMMNNNNNNNNNNNXXAGAAW where NNNNNNNNNNNXXAGAAW (N is A, G, T, or C; X can be any nucleotide; and W is A or T) has a single occurrence in the genome. For the S. pyogenes Cas9, a unique target sequence in a genome can include a Cas9 target site of the form MMMMMMMMNNNNNNNNNNNNXGGXG where NNNNNNNNNNNNXGGXG (N is A, G, T, or C; and X can be any nucleotide) has a single occurrence in the genome. A unique target sequence in a genome can include an S. pyogenes Cas9 target site of the form MMMMMMMMMNNNNNNNNNNNXGGXG where NNNNNNNNNNNXGGXG (N is A, G, T, or C; and X can be any nucleotide) has a single occurrence in the genome. In each of these sequences “M” may be A, G, T, or C, and need not be considered in identifying a sequence as unique.

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the fusion has sufficient complementarity with a tracrRNA sequence to promote one or more of: (1) excision of a guide sequence flanked by tracrRNA sequences in a cell containing the corresponding tracr sequence; and (2) formation of an endonuclease complex at a target sequence, wherein the complex comprises the crRNA sequence hybridized to the tracrRNA sequence. In general, degree of complementarity is with reference to the optimal alignment of the crRNA sequence and tracrRNA sequence, along the length of the shorter of the two sequences. Optimal alignment can be determined by any suitable alignment algorithm, and can further account for secondary structures, such as self-complementarity within either the tracrRNA sequence or crRNA sequence. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracrRNA sequence is at least 5, 6, 7, 8, 9, 10, 11, 12, 13, 14, 15, 16, 17, 18, 19, 20, 25, 30, 40, 50, or more nucleotides in length. In some embodiments, the crRNA sequence and tracrRNA sequence are contained within a single transcript, such that hybridization between the two produces a transcript having a secondary structure, such as a hairpin. In some embodiments, the loop forming sequences for use in hairpin structures are four nucleotides in length, for example, the sequence GAAA. However, longer or shorter loop sequences can be used, as can alternative sequences. The sequences preferably include a nucleotide triplet (for example, AAA), and an additional nucleotide (for example C or G). Examples of loop forming sequences include CAAA and AAAG. In one embodiment, the transcript or transcribed gRNA sequence comprises at least one hairpin. In one embodiment, the transcript or transcribed polynucleotide sequence has at least two or more hairpins. In other embodiments, the transcript has two, three, four or five hairpins. In a further embodiment, the transcript has at most five hairpins. In some embodiments, the single transcript further includes a transcription termination sequence, such as a polyT sequence, for example six T nucleotides. Non-limiting examples of single polynucleotides comprising a guide sequence, a crRNA sequence, and a tracr sequence are as follows (listed 5′ to 3′), where “N” represents a base of a guide sequence, the first block of lower case letters represent the crRNA sequence, and the second block of lower case letters represent the tracr sequence, and the final poly-T sequence represents the transcription terminator: (i) NNNNNNNNNNNNNNNNNNgtttttgtactctcaagatttaGAAAtaaatcttgcagaagctacaaagataaggctt catgccgaaatcaacaccctgtcattttatggcagggtgttttcgttatttaaTTTT; (ii) NNNNNNNNNNNNNNNNNNNNNgtttttgtactctcaGAAAthcagaagctacaaagataaggcttcatgccgaaatca acaccctgtcattttatggcagggtgttttcgttatttaaTTTTTT; (iii) NNNNNNNNNNNNNNNNNNNNNgtttttgtactctcaGAAAtgcagaagctacaaagataaggcttcatgccgaaatca acaccctgtcattttatggcagggtgtTTTTTT; (iv) NNNNNNNNNNNNNNNNNNNNgttttagagctaGAAAtagcaagttaaaataaggctagtccgttatcaacttgaaaa agtggcaccgagtcggtgcTTTTTT; (v) NNNNNNNNNNNNNNNNNNNNgttttagagctaGAAATAGcaagttaaaataaggctagtccgttatcaacttgaa aaagtTTTTTTT; and (vi) NNNNNNNNNNNNNNNNNNNNgttttagagctagAAATAGcaagttaaaataaggctagtccgttatcaTTTTT TTT. In some embodiments, sequences (i) to (iii) are used in combination with Cas9 from S. thermophilus CRISPR1. In some embodiments, sequences (iv) to (vi) are used in combination with Cas9 from S. pyogenes. In some embodiments, the tracrRNA sequence is a separate transcript from a transcript comprising the crRNA sequence.

In one embodiment, the self-targeting guide RNA comprises a palindromic sequence. In such an embodiment, the plasmid and the CRISPR/Cas9 system can be standard. For example, the targeting sequence of the guide RNA can be of the form [G-N10-reverse complement of N10] or [G-N9-reverse complement of N9]. In some embodiments, the plasmid comprises two C's that precede the guide RNA so the full sequence (using the N10 version as an example) is [CCG-N10-reverse complement of N10]. In this example, the reverse complement of this full sequence is [N10-reverse complement of N10-CGG]—this is the case because the reverse complement of a palindromic sequence is the sequence itself. Thus, once the plasmid is delivered to cells that also contain Cas9, it forms a guide RNA of the form [G-N10-reverse complement of N10], which, when complexed with Cas9, is able to recognize the [N10-reverse complement of N10-CGG] sequence in the plasmid itself. The guide RNA therefore can immediately cut the plasmid.

Broken DNA such as this cut plasmid is automatically repaired by the cell's homologous recombination machinery, and a repair template can be delivered that swaps out the palindromic guide RNA sequence for any other guide RNA targeting a genomic sequence of interest. Thus, the end result of the delivery of Cas9, palindromic guide RNA, and repair template is that cells now contain a plasmid containing a guide RNA targeting any genomic sequence of interest.

### Homologous Recombination/Repair Templates

In some embodiments, a recombination template or “repair” template is also provided. A repair template can be a component of another vector as described herein, contained in a separate vector, or provided as a separate polynucleotide. The repair template can be either single-stranded or double-stranded DNA. In some embodiments, a repair template is designed to serve as a template in homologous recombination, such as within or near a target sequence nicked or cleaved by an RNA-guided endonuclease, such as a CRISPR enzyme as a part of a CRISPR complex. A template polynucleotide can be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide can overlap with one or more nucleotides of a target sequence (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In one embodiment, the homology arms of the repair template are directional (i.e., not identical and therefore bind to the sequence in a particular orientation).

### Codon Optimization

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

### Delivery of Nucleic Acid Sequences to Cells

In some aspects, the methods provided herein comprise delivering one or more polynucleotides, such as or one or more vectors/plasmids as described herein, one or more transcripts thereof, and/or one or more proteins transcribed therefrom, to a host cell. Also provided herein are cells produced by such methods, and organisms (such as animals, plants, or fungi) comprising or produced from such cells.

In some embodiments, an RNA-guided endonuclease in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of an RNA-guided endonuclease system to cells in culture, or in a host organism.

Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In one embodiment, the nucleic acids described herein are administered to a cell by transfection. Transfection methods useful for the methods described herein include, but are not limited to, lipid-mediated transfection, cationic polymer-mediated transfection, or calcium phosphate precipitation.

In another embodiment, the nucleic acids described herein are administered to a cell by electroporation (e.g., nucleofection).

In another embodiment, the nucleic acids described herein are administered to a cell by means of a viral vector, including adenoviral or retroviral (e.g., lentiviral) vectors.

Exemplary methods for introducing nucleic acid compositions for use in genome modification can be found in e.g., Mali et al. “RNA-guided human genome engineering with Cas9” Science (2013) 339:823-26; Dicarlo et al. “Genome engineering in Saccharomyces cerevisiae using CRISPR-Cas systems” Nucleic Acids Research (2013) 7:4336-43; Esvelt et al. “Orthogonal Cas9 proteins for RNA-guided genome regulation and editing” Nat Methods (2013) 10:1116-21; Jao et al. “Efficient multiplex biallelic zebrafish genome editing using a CRISPR nuclease system” Proc Natl Acad Sci (2013) 110:13904-9; Ding et al. “Enhanced Efficiency of Human Pluripotent Stem Cell Genome Editing through Replacing TALENs with CRISPRs” Cell Stem Cell (2013) 12(4):393-4, among others.

The present invention may be as defined in any one of the following numbered paragraphs:

1. A method of generating a plasmid intracellularly for targeted modification of a genomic sequence, the method comprising introducing to the cell: (a) an expression construct encoding an RNA-guided endonuclease; and (b) a plasmid encoding a sequence directing the transcription of a self-targeting RNA guide sequence, comprising a self-targeting sequence, wherein the self-targeting RNA forms a complex with the RNA-guided endonuclease to initiate cleavage of a self-targeted sequence in the plasmid sequence encoding the self-targeting RNA guide sequence, such that transcription of the self-targeting RNA in the presence of the RNA-guided endonuclease permits the formation of a complex with the RNA guided endonuclease that directs the cleavage of the plasmid within the self-targeted sequence; and (c) a repair template comprising a genomic targeting sequence flanked by first and second homology arms homologous, respectively, to sequences that flank said self-targeting sequence in the plasmid, the genomic targeting sequence sufficient to direct cleavage of an associated RNA-guided nuclease to a genomic target sequence, wherein, upon introduction of the expression construct encoding an RNA-guided endonuclease, the plasmid and the repair template to the cell, the plasmid is cleaved in the self-targeted sequence and the repair template comprising the genomic targeting sequence directs the homologous replacement of the self-targeted sequence with the genomic targeting sequence, whereby the cleavage-guiding specificity of the self-targeted guide RNA is modified to the genomic target sequence.

2. The method of paragraph 1, wherein expressed RNA-guided endonuclease forms a complex with the modified guide RNA expressed from the plasmid and wherein the complex with modified guide RNA effects targeted modification of the genomic target sequence.

3. The method of paragraph 1 or 2, wherein the expression construct is a plasmid.

4. The method of any one of paragraphs 1-3, wherein the endonuclease is a Cas endonuclease.

5. The method of paragraph 4, wherein the Cas endonuclease is Cas9.

6. The method of paragraph 2, wherein the RNA-guided endonuclease introduces a double-stranded break in the genomic target sequence.

7. The method of paragraph 2, wherein the method does not require cloning of a sequence into a cloning vector.

8. The method of paragraph 2, further comprising providing a linear repair template for homologous recombination-mediated repair at the selected genomic target sequence.

9. The method of paragraph 8, wherein the process of homologous recombination inactivates the target sequence.

10. The method of paragraph 8, wherein the repair template comprises an engineered DNA sequence flanked by first and second homology arms homologous, respectively, to sequences that flank the selected genomic targeting sequence.

11. The method of paragraph 10, wherein the engineered DNA sequence comprises a sequence encoding one or more nucleotide mutation(s), one or more inserted nucleotide(s), or one or more deleted nucleotide(s).

12. The method of any one of paragraphs 1-11, wherein each of the self-targeted guide RNA sequence and the guide RNA expressed from the modified plasmid comprises at least one hairpin.

13. The method of any one of paragraphs 1-12, wherein each of the self-targeted guide RNA sequence and the guide RNA expressed from the modified plasmid comprises a crRNA and/or a tracrRNA sequence to permit association of the guide RNA with the RNA-guided endonuclease.

14. The method of any one of paragraphs 1-13, wherein the crRNA and/or tracrRNA sequence is codon optimized for the organism comprising the selected genomic target sequence.

15. The method of paragraph 13, wherein the crRNA and tracrRNA sequence comprise a fusion sequence.

16. The method of any one of paragraphs 1-15, wherein the expression construct and the plasmid are introduced to the cell by electroporation, transfection, or viral delivery.

17. The method of any one of paragraphs 1-16, wherein the expression construct or the plasmid further comprises a sequence encoding a reporter molecule.

18. The method of paragraph 17, wherein the reporter molecule is GFP.

19. The method of any one of paragraphs 1-18, wherein the self-targeting sequence comprises a palindromic sequence.

20. A composition comprising a nucleic acid vector encoding a sequence directing the transcription of a self-targeting RNA guide molecule for an RNA-guided endonuclease, the sequence comprising a self-targeting sequence, wherein when contacted with the RNA guided endonuclease, self-targeting RNA guide molecule transcribed from the vector forms a complex with the RNA-guided endonuclease, and wherein the complex cleaves the plasmid in the sequence encoding the self-targeting RNA guide molecule, such that transcription of the self-targeting RNA in the presence of the RNA-guided endonuclease results in cleavage of the nucleic acid vector in the sequence encoding the self-targeting RNA guide molecule.

21. The composition of paragraph 20, wherein the nucleic acid vector further encodes an RNA-guided endonuclease.

22. The composition of paragraph 20 or 21, wherein the endonuclease is a Cas endonuclease.

23. The composition of paragraph 22, wherein the Cas endonuclease is Cas9.

24. The composition of any one of paragraphs 20-23, wherein the RNA-guided endonuclease introduces a double-stranded break in the targeted sequence.

25. The composition of any one of paragraphs 20-24, wherein the self-targeting RNA guide molecule comprises at least one hairpin.

26. The composition of any one of paragraphs 20-25, wherein the self-targeting RNA guide molecule comprises a crRNA and/or a tracrRNA sequence to permit association of the guide RNA with the RNA-guided endonuclease.

27. The composition of any one of paragraphs 20-26, wherein the crRNA and/or tracrRNA sequence is codon optimized for an organism in which targeted modification of the genome is desired.

28. The composition of any one of paragraphs 20-27, wherein the crRNA and tracrRNA sequence comprise a fusion sequence.

29. The composition of any one of paragraphs 20-28, wherein the self-targeting sequence comprises a palindromic sequence.

30. A composition comprising the composition of any one of paragraphs 20-29 and a linear repair template comprising a genomic targeting sequence, flanked by first and second homology arms homologous, respectively, to sequences that flank the self-targeting sequence in the vector.

31. A kit comprising the composition of any one of paragraphs 20-30 and instructions therefor.

32. The kit of paragraph 31, further comprising an expression construct encoding an RNA-guided endonuclease.

33. A cell comprising a composition of any one of claims 20-30.

34. The cell of paragraph33, wherein the cell is a mammalian cell, a plant cell, an insect cell, or a cell of a pathogen or pest.

35. The cell of paragraph33, wherein the cell is a human cell.

36. The cell of paragraph33, wherein the cell is a cancer cell.

### EXAMPLES

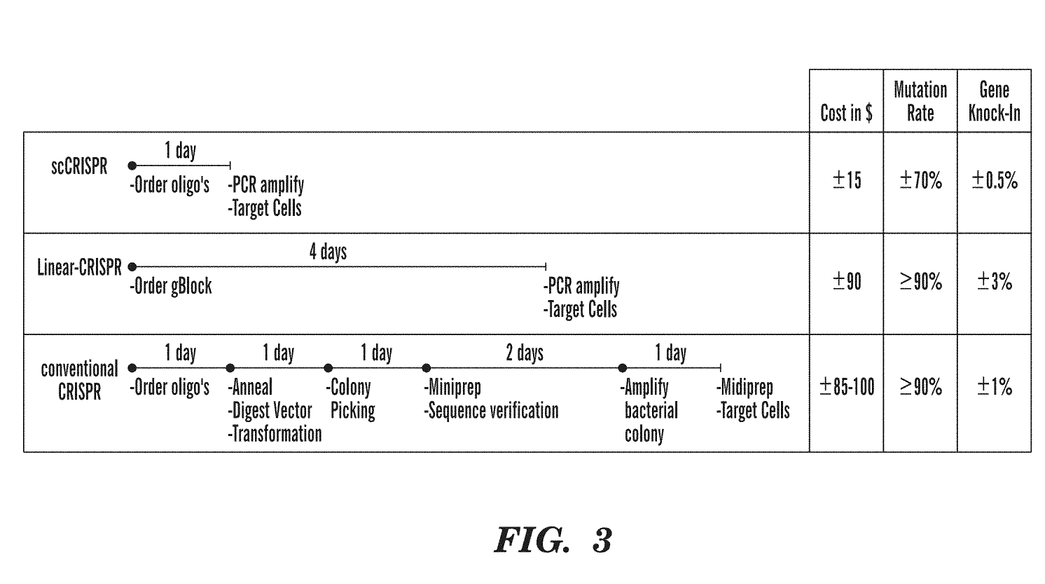

Currently, CRISPR targeting still requires molecular cloning of a site-specific sgRNA plasmid for every new locus, which involves the time-consuming and costly steps of plasmid ligation, transformation, purification, and sequence verification over the course of about one week. This investment hinders large-scale sgRNA screening necessary for complexed and high throughput genome editing applications. Additionally, knock-in transgenesis of genes such as GFP using CRISPR still requires the time-consuming construction of homology constructs typically with 600-6000 bp homology arms, laborious steps that impede routine knock-in line generation. These barriers are holding back the revolutionary potential of large scale targeted genome manipulation. Provided herein are alternative methods of sgRNA and homology construct construction that eliminate the need for plasmid cloning and thus substantially reduce the time, workload, and cost of CRISPR-mediated genome editing while maintaining high efficiency of site-specific mutation and improving on transgene insertion into mouse and human embryonic stem cells (FIG. 3).

In the standard CRISPR method, once a site-specific sgRNA sequence is found, it is cloned into a plasmid containing a hairpin structure enabling Cas9 binding and a U6 promoter capable of transcribing the sgRNA hairpin in target cells.^{3,4} As each locus to be targeted requires a unique sgRNA sequence, this plasmid cloning step must be performed for every new sgRNA to be used, providing a major bottleneck to the throughput of CRISPR-mediated genome editing. The inventors have designed two methods that circumvent any cloning steps in the gene editing process and demonstrate efficacy at genome editing in both mouse and human cells. These methods vastly simplify the generation of targeted transgenic or knockout cell lines without compromising in genome editing efficiency. In addition to the generation of mutation- and reporter cell models, the technologies presented here open up new avenues in mutation screening applications.

One of the most transformative applications of CRISPR is the generation of gene knock-ins through site-specific homologous recombination. Essentially any sequence can be introduced by knock-in; fluorescent report knock-in provides proof of principle an can also be of practical use, e.g., for studies of gene expression, cell fate, or development, among others. Traditional knock-in creation utilizing CRISPR requires the construction of a plasmid homology template with 600-6000 bp homology arms flanking the insert sequence, a laborious undertaking requiring 1-2 weeks of molecular cloning for each targeted site, severely limiting the throughput of knock-in generation. In the traditional approach, a gene-specific sgRNA plasmid (which must also be constructed), Cas9, and the plasmid homology template are co-electroporated into target cells, and screening is performed to purify the small percentage of clones that have undergone successful knock-in. The inventors asked whether they could perform plasmid-free GFP knock-in by using Linear-CRISPR and flanking GFP with short homology arms added through tailed PCR.

### Example 1: Linear CRISPR

Demonstration of Target Gene Knock-Out Via Linear CRISPR

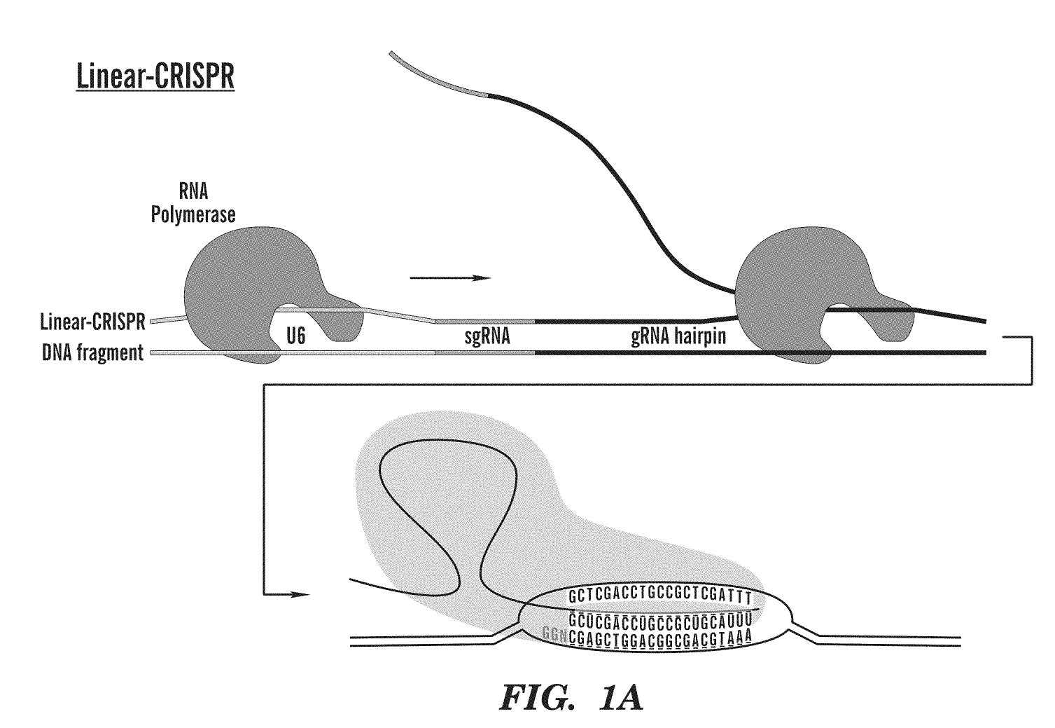

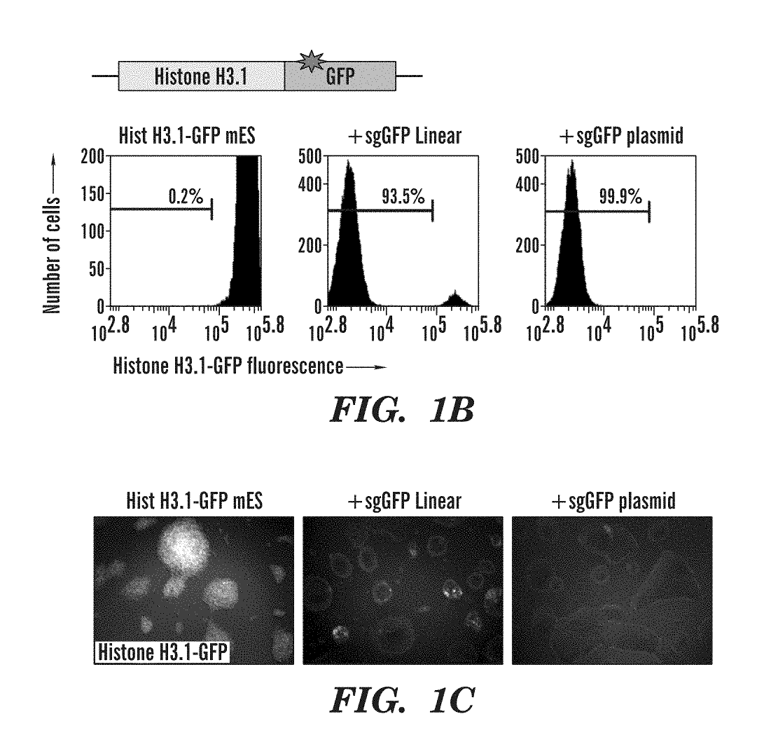

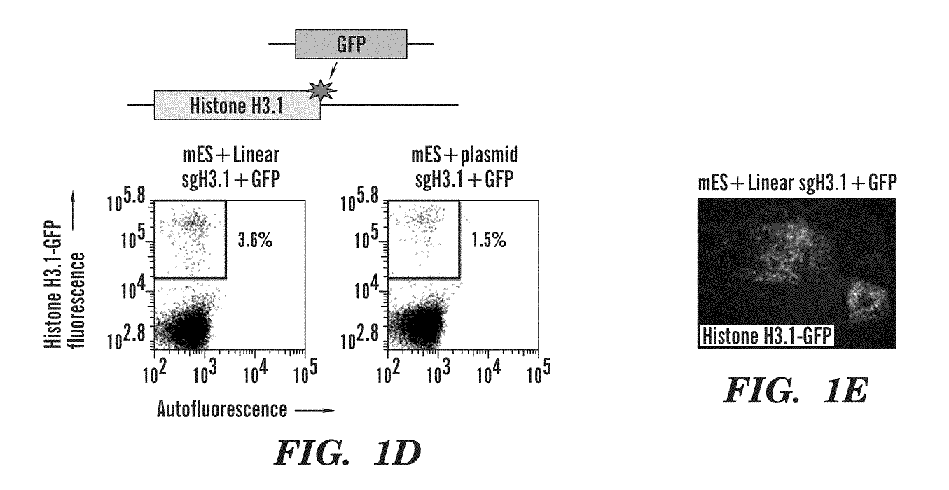

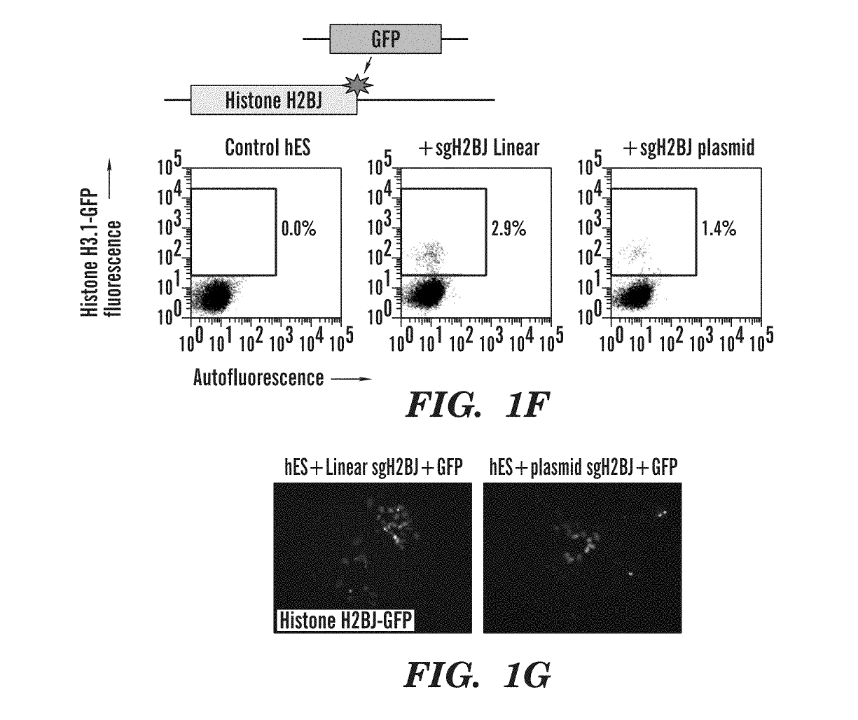

Assembled custom DNA fragments are readily available for purchase at relatively low cost. The inventors explored whether a 500 bp Linear-CRISPR DNA fragment including a U6 promoter, GFP-targeting sgRNA sequence, and gRNA hairpin sequence could substitute for an sgRNA plasmid. The inventors ordered and PCR-amplified a 500 bp Linear-CRISPR targeting GFP (FIG. 1A) and co-electroporated it into Histone H3.1 (Hist1h3a)-GFP knock-in mouse embryonic stem cells (mESCs) along with a Cas9 expression plasmid that encodes Blasticidin resistance, allowing antibiotic selection to enrich for cells that received Cas9. The GFP targeting Linear-CRISPR knocked out GFP fluorescence in 93.5% of targeted cells (FIGS. 1B, 1C), equivalent to the standard plasmid sgRNA CRISPR method (99.9%).

Demonstration of Target Gene Knock-in Via Linear CRISPR

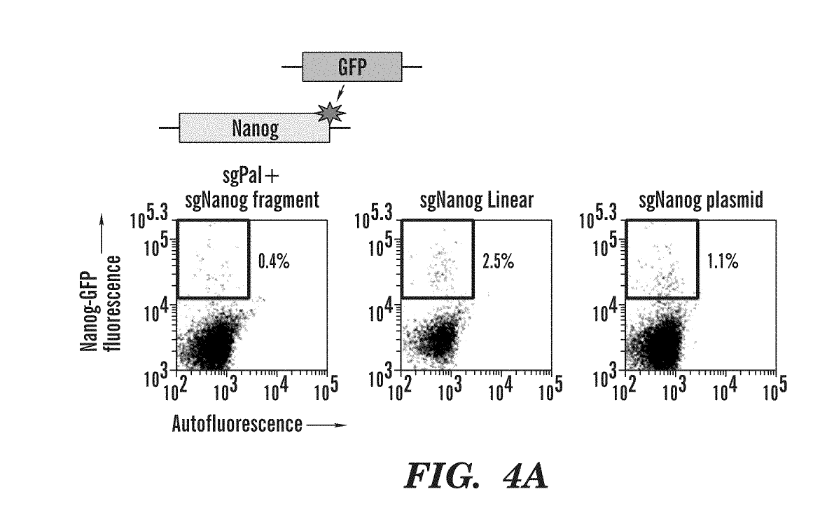

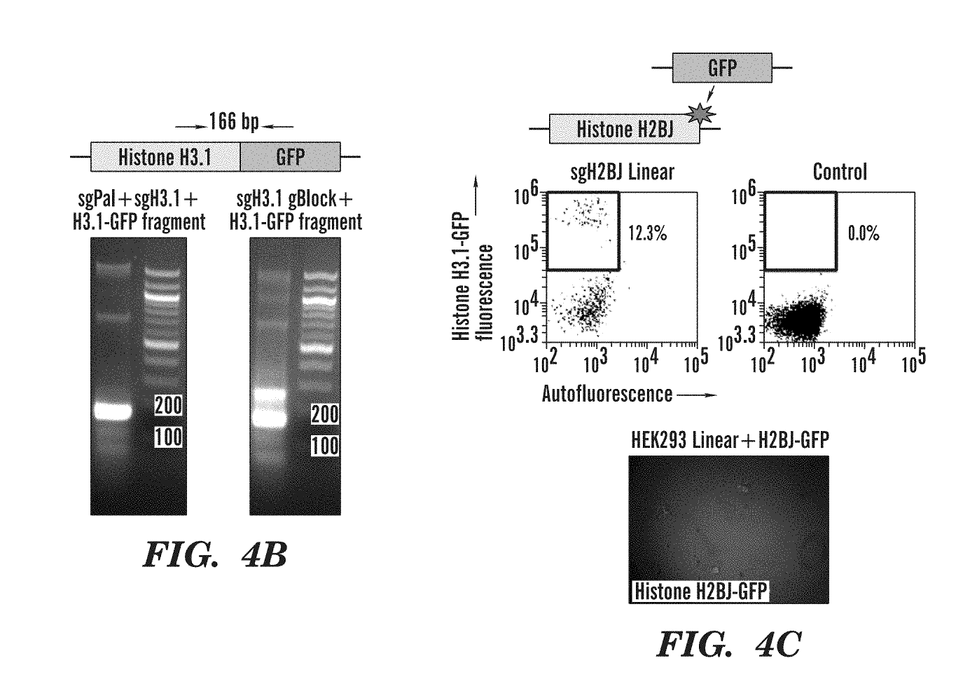

To perform plasmid-free GFP knock-in, the inventors designed an sgRNA targeting the C-terminus of the Histone H3.1 gene in wildtype mESCs and performed PCR to generate a GFP homology template with 80 bp of Histone H3.1 homology sequence on either side of GFP which should produce a C-terminal GFP fusion protein when recombined into the genome. To test PCR-based GFP knock-in, the inventors co-electroporated Cas9, Histone H3.1-targeting sgRNA plasmid, and Histone H3.1-GFP homology template fragment into mESCs. One week after electroporation, 1.5% of cells expressed strong nuclear GFP and showed site-specific GFP integration by genomic DNA PCR (FIG. 1D, and FIG. 4). Similar results were achieved constructing a Nanog-GFP knock-in mESC line (1.1%, FIG. 4). Thus, PCR-based gene knock-in presents an effective method of generating transgenic mESC lines. Given the high efficiency of Linear-CRISPR GFP mutation, the inventors performed a wholly plasmid-free GFP knock-in using Linear-CRISPR genome targeting and a PCR-based GFP homology fragment. A 3.5% GFP knock-in at the Histone H3.1 locus and 2.5% Nanog-GFP knock-in (FIGS. 1D, 1E, and FIG. 4) was achieved.

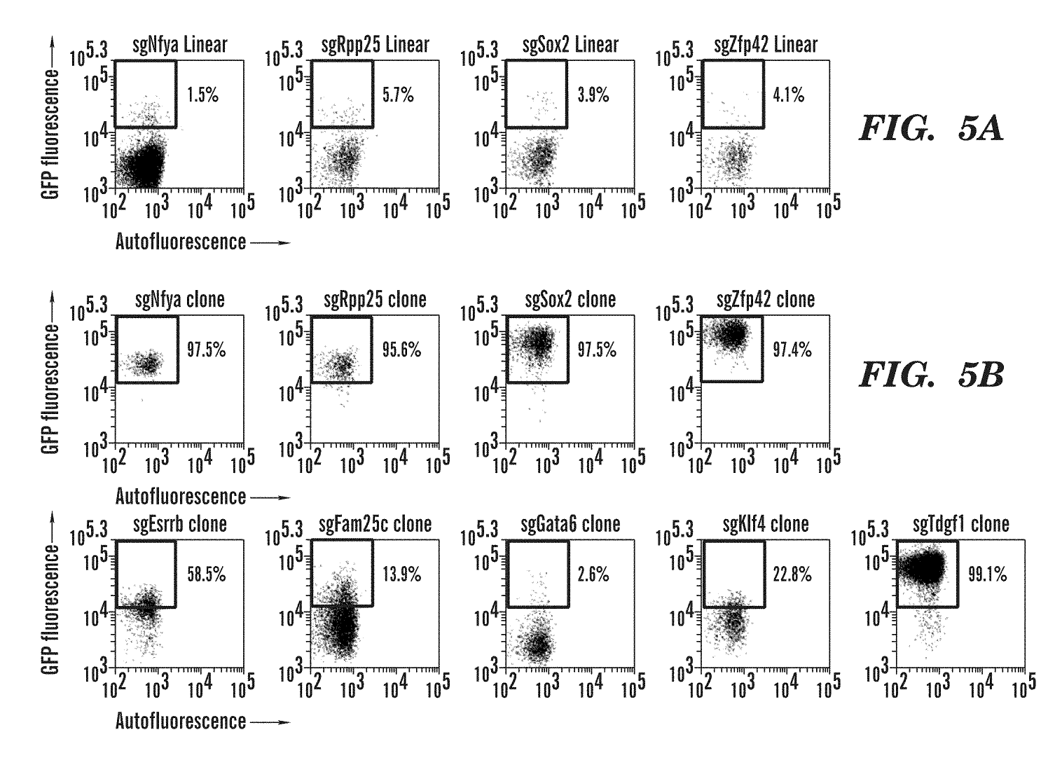

To demonstrate the reproducibility of cloning-free Linear-CRISPR-based mESC knock-in generation, the inventors constructed nine additional site-specific GFP knock-in lines including C-terminal GFP fusion lines in the Esrrb, Fam25c, Gata6, Klf4, Nfya, Rpp25, and Sox2 loci and GFP replacements in the Tdgf1 and Zfp42 loci (FIG. 5), successfully deriving clonal knock-in lines. Thus, the inventors show that genomic knock-in can be performed without any molecular cloning at equivalent or enhanced efficiency to the traditional plasmid-based approach. This approach dramatically decreases the time, cost, and labor involved in transgenesis.

The inventors explored whether Linear-CRISPR performs just as efficiently in human embryonic stem cells (hESC), for which knock-in line generation has traditionally been prohibitively difficult. The inventors designed a Linear-CRISPR targeting the C-terminus of the human Histone H2BJ locus. Co-transfection into HUES2 hESCs together with Cas9 and a PCR amplified GFP homology fragment yielded efficient (2.9%) H2BJ-GFP fusion in human ES cells (FIGS. 1F, 1G), compared to 1.4% of conventional plasmid targeted cells. Linear-CRISPR also yielded highly efficient (12%) H2BJ-GFP gene insertion in the commonly used human embryonic kidney cell line 293T (HEK293T) (FIG. 4). Thus, presented herein is an approach that allows efficient construction of human ES cell knock-in lines with a total of two hours of preparation time, a finding that allows for a substantial increase in the throughput of human ES knock-in line generation.

Linear-CRISPR requires minimal effort and is optimally suited for both gene editing and transgenic applications of a defined set of target genes. Yet, in high-throughput applications where many individual sgRNAs must be tested, the cost of DNA fragment synthesis, as with molecular cloning, remains limiting.

### Example 2: Self-Cloning CRISPR

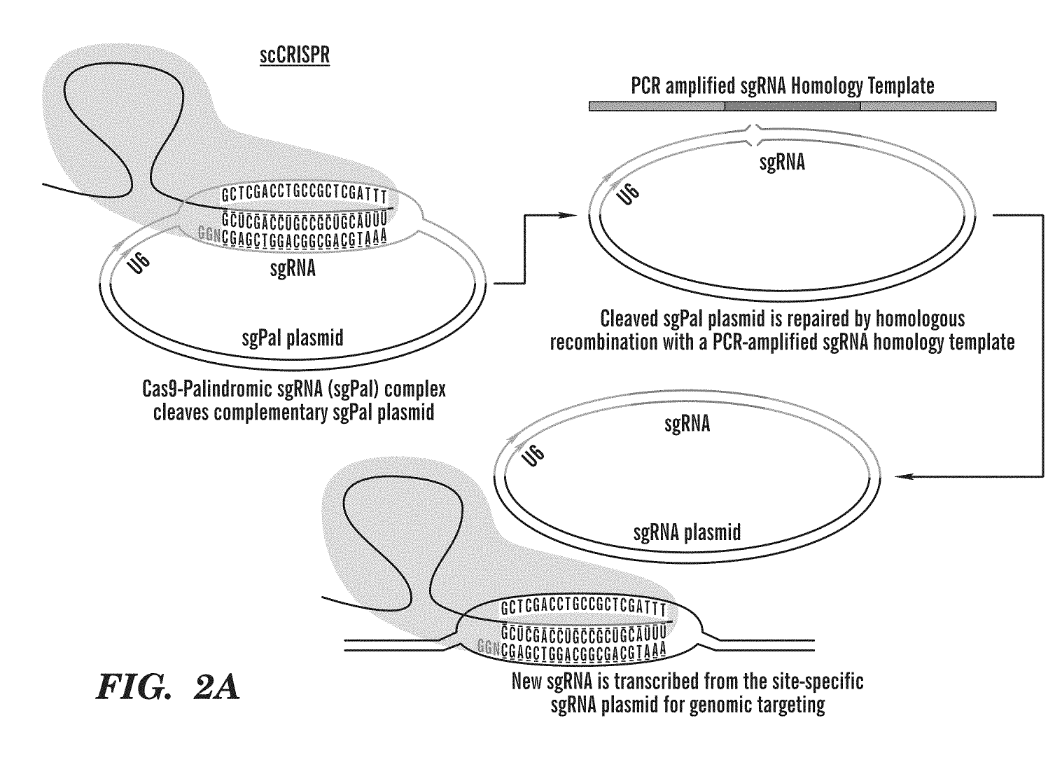

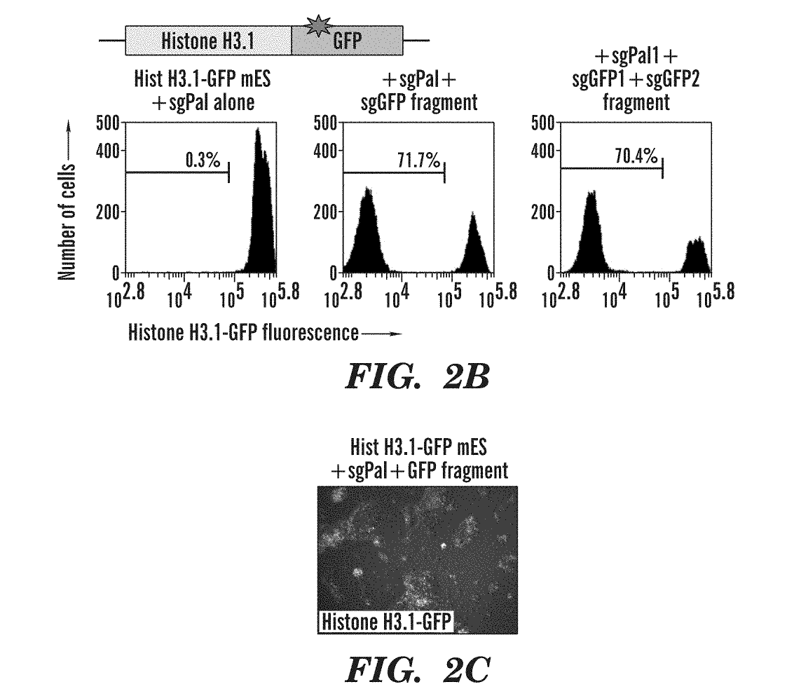

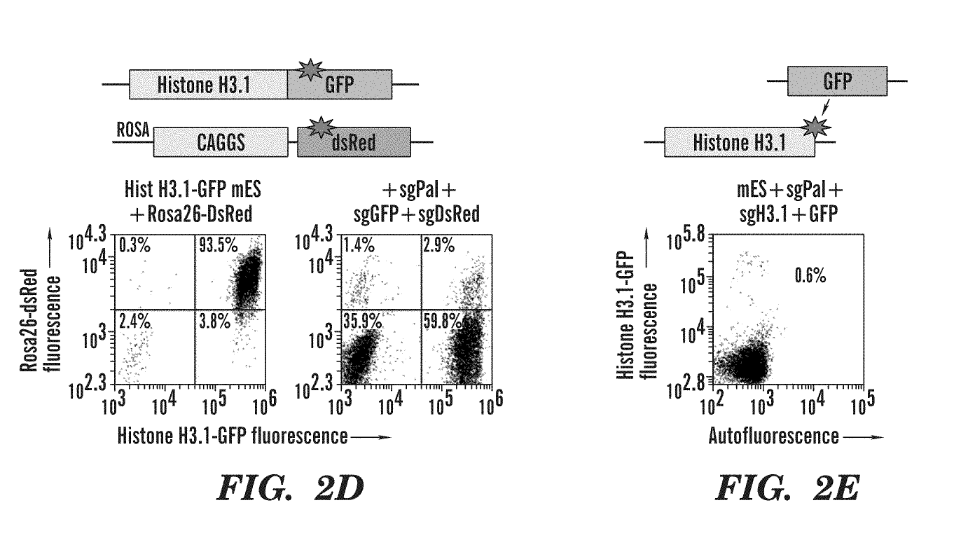

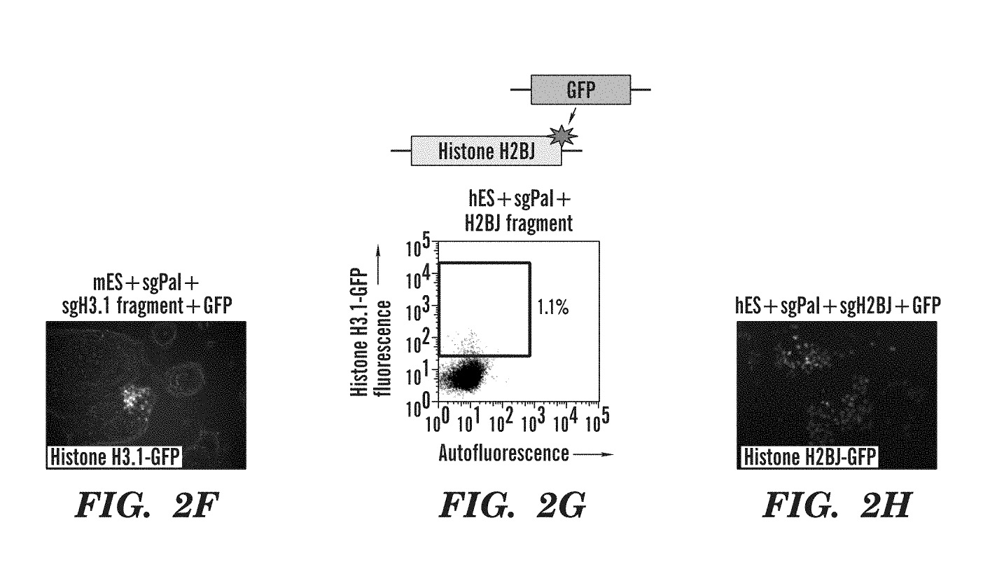

The inventors thus present a second, improved and more affordable technology for high-throughput gene editing applications that avoids the need to construct locus specific sgRNA vectors for genomic targeting. Self-Cloning CRISPR (scCRISPR) relies on the target cells to “clone” the desired sgRNA sequence. Mammalian cells are known to repair introduced plasmid DNA through homologous recombination (HR)⁵⁻⁷. It was asked whether one could take advantage of plasmid HR by introducing a template sgRNA plasmid into cells that could be recombined with a small DNA fragment containing the desired site-specific sgRNA sequence to form a functional site-specific sgRNA plasmid. The HR pathway is stimulated by double-stranded DNA breaks⁸, so a self-cleaving palindromic template sgRNA plasmid was designed that, upon transcription in cells, would induce a DNA break in its own sequence which is subsequently repaired into a functional site-specific sgRNA (FIG. 2A).

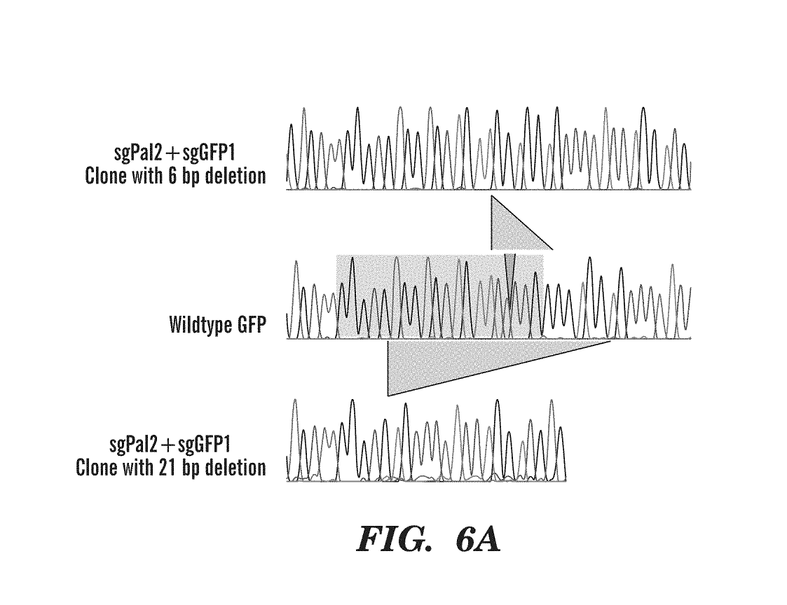

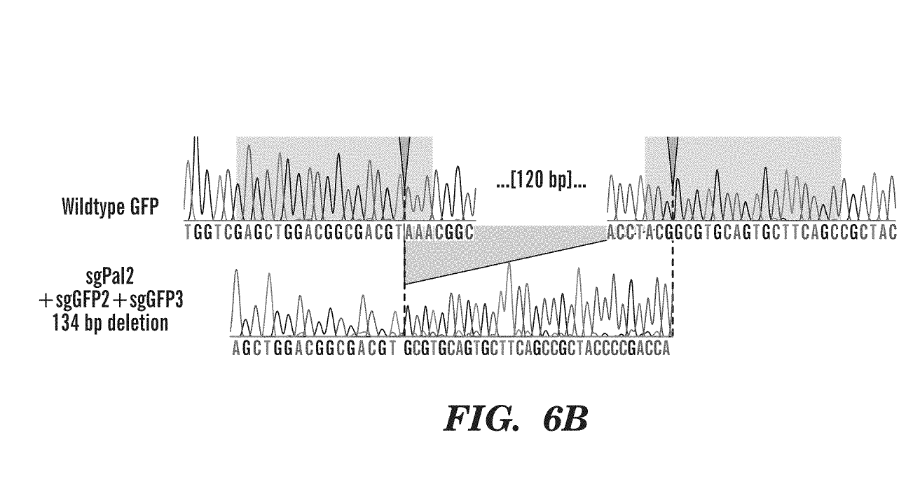

To implement scCRISPR, the inventors designed self-complementary palindromic sgRNA plasmids (sgPals) that induce their own cleavage after complexing with Cas9 in cells. To minimize off-target genomic DNA cleavage by sgPal, the inventors designed four sgPal sequences with minimal predicted off-target cleavage potential (Table 1). The inventors also designed an oligonucleotide that, upon PCR amplification, contains an sgRNA sequence targeting green fluorescent protein (GFP) flanked by 120 bp of homology to the sgPal plasmid on either side (FIG. 2A). The inventors co-electroporated a Cas9 expression plasmid, an sgPal plasmid, and the GFP-targeting sgRNA homology fragment into Histone H3.1 (Hist1h3a)-GFP knock-in mESCs. The Cas9 plasmid encodes Blasticidin resistance and the sgPal plasmid encodes Hygromycin resistance, allowing antibiotic selection to enrich for cells that received both plasmids. Electroporation of sgPal, Cas9, and a GFP-targeting sgRNA homology fragment induced loss of GFP in 72% of cells one week after electroporation, while Cas9 and sgPal alone with no GFP-targeting sgRNA homology fragment produced minimal detectable GFP loss (0.3%) (FIG. 2B). Sequence analysis confirmed loss of GFP was a result of genomic mutations at and around the target site of the sgGFP fragment (FIG. 6). Thus, scCRISPR is an efficient method of inducing site specific genomic mutation, producing target gene (here, the test target, GFP) loss in a majority of cells.

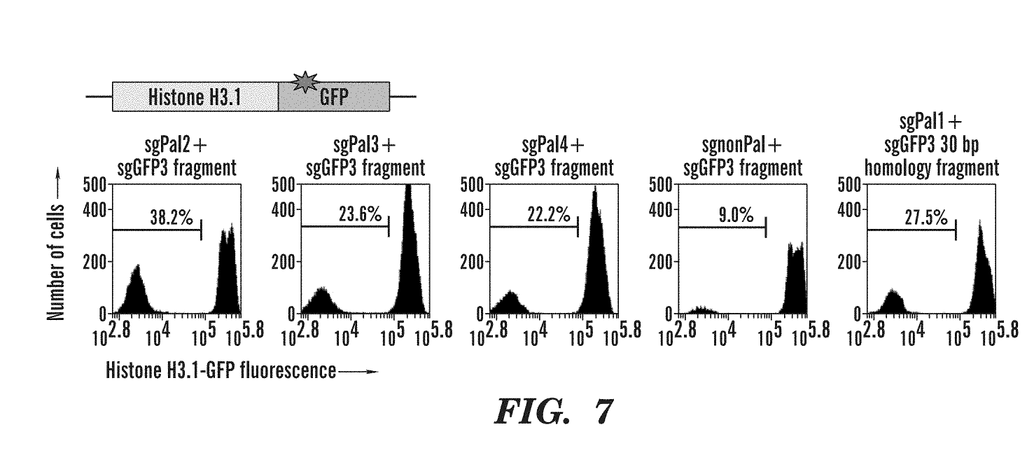

To determine whether scCRISPR indeed functions through plasmid HR, the inventors varied the sgRNA plasmid and HR donor fragments. The inventors found that all four of the sgPal plasmids designed induced substantial GFP loss (72%, 38%, 23%, 22%), while substituting the sgPal plasmid with a non-self-cleaving sgRNA plasmid produced only 9% GFP loss (FIG. 7). The difference in efficiency between the distinct sgPals for GFP mutation may be due to sequence characteristics affecting CRISPR cleavaged.⁹ While overall efficiency may vary somewhat, it is well within the abilities of one of skill in the art to perform, and, indeed, optimize the methods with a minimum of experimentation. For the remainder of the study, only the most efficient of the four, sgPal1, was used. Without wishing to be bound by theory, the inventors speculate that the enhanced rate of GFP loss with a non-self-cleaving sgRNA (9%) as compared to controls without any homology fragment (0.5%) is the result of plasmid HR occurring in the absence of a double-strand break.

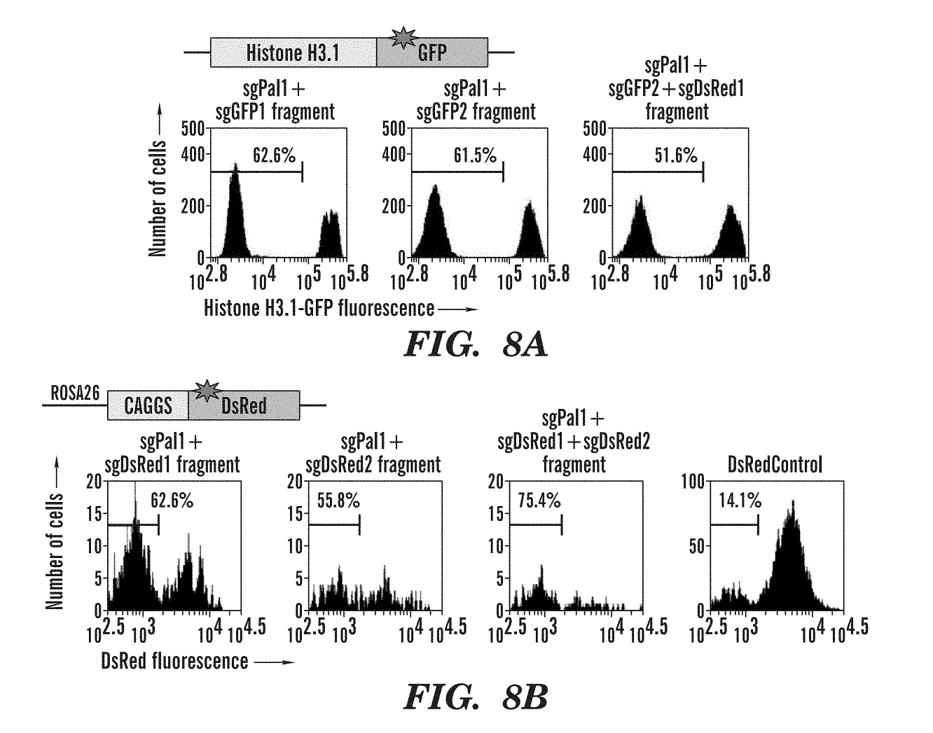

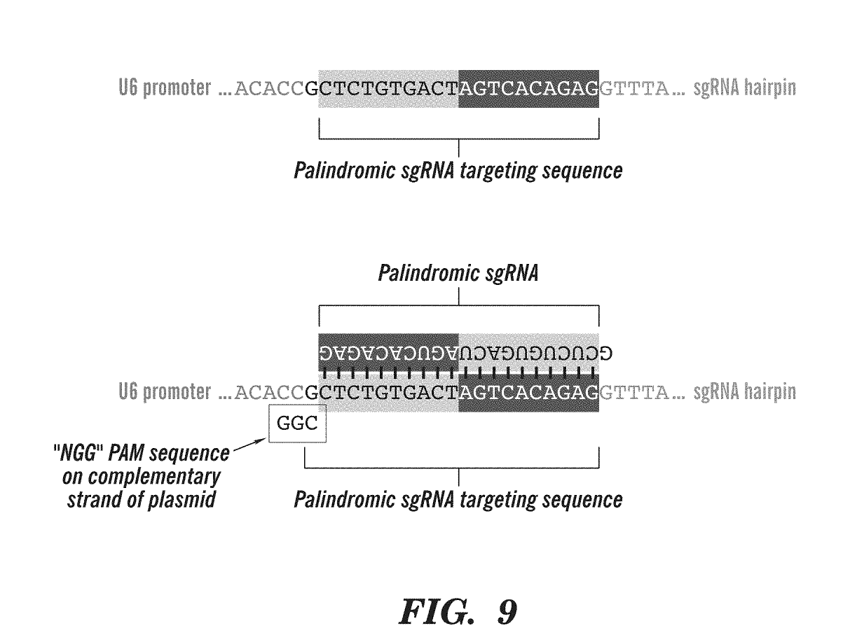

The inventors also varied the length of homology in the sgRNA homology fragment, finding that decreasing from 120 to 30 bp of homology decreased the GFP loss after recombination with sgPal1 to 27% (FIG. 7). Thus, scCRISPR is optimally efficient with a self-cleaving sgRNA donor and an sgRNA acceptor with long homology arms, providing evidence that plasmid HR is required for scCRISPR. It was next asked if the homologous recombination of sgPal plasmids in cells occurs at a high enough frequency to target multiple sites in a single experiment. The inventors designed sgRNA homology fragments targeting two additional locations within GFP and two within dsRed. All four additional sgRNAs produced >50% loss of GFP or dsRed (FIG. 8) in Histone H3.1-GFP or Rosa26-CAGGS-dsRed cells respectively, indicating that scCRISPR works with a variety of sgRNAs. The inventors then introduced two sgRNAs simultaneously into mESCs, finding high rates of GFP loss with two GFP-targeting sgRNAs in Histone H3.1-GFP cells (70%, FIG. 2B), two dsRed targeting oligos in Rosa26-CAGGS-dsRed cells (75%), or one GFP-targeting and one off-target dsRed-targeting sgRNA in Histone H3.1-GFP cells (51% loss of GFP, FIG. 8). Dual targeting with GFP-targeting sgRNAs led to deletion mutations as opposed to indels induced by single-targeted scCRISPR (FIG. 6), indicating that scCRISPR allows efficient complexed sgRNA genome editing.

To more rigorously assess the capability to multiplex sgRNAs in scCRISPR, both GFP and dsRed were targeted simultaneously in Hist1h3a-GFP Rosa26-dsRed double positive mESCs by co-electroporation of sgPal1, Cas9, and two separate sgRNA homology fragments targeting GFP and dsRed. Consistent with the targeting efficiency of the individual sgRNAs against GFP and DsRed, fluorescence of GFP and DsRed was lost in 37.3% and 95.7% of cells respectively, with both genes knocked out in 35.9% of cells (FIG. 1D). Thus, scCRISPR is well suited to study the effects of compound mutations by simultaneous genome editing at multiple genomic loci in parallel.

The inventors carried out scCRISPR plasmid-free GFP knock-in by co-electroporating Cas9, sgPal1, Histone H3.1-targeting sgRNA homology fragment, and Histone H3.1-GFP homology template fragment into mESCs. One week after electroporation, 0.6% of cells expressed strong nuclear GFP and showed site-specific GFP integration by genomic DNA PCR (FIGS. 2E, 2F, 4). The inventors achieved similar results constructing a Nanog-GFP knock-in mESC line (0.4%, FIG. 4). Likewise, we co-electroporated HUES2 with Cas9, sgPal1, Histone H2BJ-targeting homology fragment, and Histone H2BJGFP homology template fragment to knock-in nuclear GFP expression in hESCs. Fourteen days after electroporation, 1.1% of cells express GFP fluorescence (FIGS. 2G, 2H), equivalent to targeting with conventional plasmid CRISPR, and mESC targeted gene insertion efficiencies. Thus, scCRISPR presents a simple, quick, inexpensive, and highly effective method of generating GFP knock-in both mESC and hESC lines without any plasmid cloning.

The inventors present Linear-CRISPR as an optimized CRISPR technology, equal or superior to plasmid-based methods for targeted genome mutations and transgenesis in hESC and mESC, while reducing both time and expense (FIG. 3). Still, DNA fragments take several days longer to construct and are more expensive than the shorter oligonucleotides required for scCRISPR (FIG. 3); therefore, scCRISPR permits rapid (3 hours from oligonucleotide arrival vs. 6 days for conventional CRISPR) and cost-effective (˜⅙ the cost) application of CRISPR when non-uniform mutation or knock-in frequency can be tolerated. These include cases in which mutant clones will be picked or purified and sequenced or cases in which multiple sgRNAs will be screened or multiplexed to choose the most effective for a given application. Linear-CRISPR is indicated for use when near-uniform targeting efficiency or maximal knock-in efficiency is required. Both scCRISPR and Linear-CRISPR methodologies advance CRISPR technology by substantially reducing the effort and increasing the throughput of CRISPR-mediated genomic mutation, and importantly of gene knock-in in mouse and human cell lines. By eliminating molecular cloning, these methods lower the bar for targeted genome editing, opening up opportunities for novel high-throughput genome editing and knock-in screening applications.

### REFERENCES

1. Cong L, Ran F A, Cox D, et al. Multiplex genome engineering using CRISPR/Cas systems. Science. 2013; 339(6121):819-823.
2. Mali P, Yang L, Esvelt K M, et al. RNA-guided human genome engineering via Cas9. Science. 2013; 339(6121):823-826.
3. Ran F A, Hsu P D, Wright J, Agarwala V, Scott D A, Zhang F. Genome engineering using the CRISPRCas9 system. Nat Protoc. 2013; 8(11):2281-2308.
4. Yang L, Yang J L, Byrne S, Pan J, Church G M. CRISPR/Cas9-Directed Genome Editing of Cultured Cells. Curr Protoc Mol Biol. 2014; 107:31.31.31-31.31.17.
5. Small J, Scangos G. Recombination during gene transfer into mouse cells can restore the function of deleted genes. Science. 1983; 219(4581): 174-176.
6. Folger K R, Wong E A, Wahl G, Capecchi M R. Patterns of integration of DNA microinjected into cultured mammalian cells: evidence for homologous recombination between injected plasmid DNA molecules. Mol Cell Biol. 1982; 2(11): 1372-1387.
7. Wake C T, Wilson J H. Simian virus 40 recombinants are produced at high frequency during infection with genetically mixed oligomeric DNA. Proc Natl Acad Sci USA. 1979; 76(6):2876-2880.
8. Rouet P, Smih F, Jasin M. Expression of a site-specific endonuclease stimulates homologous recombination in mammalian cells. Proc Natl Acad Sci USA. 1994; 91(13):6064-6068.
9. Ren X, Yang Z, Xu J, et al. Enhanced Specificity and Efficiency of the CRISPR/Cas9 System with Optimized sgRNA Parameters in Drosophila. Cell Rep. 2014; 9(3): 1151-1162.

### Methods for Examples 1 & 2

Cell Culture

Mouse embryonic stem cell culture was performed according to previously published protocols³¹. All experiments were performed with 129P2/OlaHsd mouse ES cells except for the DsRed targeting which was performed using the IB10 mESC line. mESCs were maintained on gelatin-coated plates feeder-free in mES media composed of Knockout DMEM (Life Technologies) supplemented with 15% defined fetal bovine serum (FBS) (HyClone), 0.1 mM nonessential amino acids (NEAA) (Life Technologies), Glutamax (GM) (Life Technologies), 0.55 mM 2-mercaptoethanol (b-ME) (Sigma), 1×ESGRO LIF (Millipore), 5 nM GSK-3 inhibitor XV and 500 nM UO126. Cells were regularly tested for mycoplasma.

Histone H3.1-GFP fusion mESCs were created using the gBlock-CRISPR method described in this work and cloned such that >99.5% of cells expressed strong nuclear GFP. ROSA-CAGGS-DsRed IB10 mESCs were created using plasmid-based knock-in and also cloned to enrich for DsRed-expressing cells.

HEK293FT cells were cultured using DMEM (Life Technologies) supplemented with 10% FBS (HyClone). Human embryonic stem cell culture was performed according to previously published protocols. All experiments were performed with HUES2 human ES cells. hESCs were maintained on gelatin coated plates on an feeder layer of irradiated murine embryonic fibroblasts (MEFs) in complete hES media composed of 1:1 DMEM:F12 (Life Technologies) supplemented with 15% KOSR, 0.1 mM NEAA (Life Technologies), GM (Life Technologies), 3.2 mM b-ME (Sigma), 20 ng/ml bFGF (R&D Systems), 5 nM GSK-3 inhibitor XV and 500 nM U0126. Cells were regularly tested for mycoplasma.

Prior to electroporation, hESCs were enzymatically passaged using 0.25% trypsin and quenched with complete hES media supplemented with 1% FBS (HyClone) and 10 uM uM Y-27632 (Tocris). To deplete the cell suspension of feeders, the cells were plated onto a 15 cm dish in 7 ml quenching media and incubated at 37° C. for 30 min. The media was then carefully transferred to a 15 ml tube and pelleted to remove excess serum.

scCRISPR Off-Target Effect Analysis

To use CRISPR for genome editing, a site-specific sgRNA sequence must be designed by a set of rules that determine both the efficiency and specificity of CRISPR targeting. sgRNAs comprise a targeting sequence that is typically 20 bp long although 17-21 bp sgRNAs have been reported to be functional²⁻⁵. Cas9 will recognize and cleave DNA only when there is a PAM-sequence (-NGG) in the genome that is directly 3′ of the sgRNA sequence⁶⁻⁸.

Lastly, Cas9 can generate off-target DNA cleavage at sites bearing close similarity to the sgRNA targeting sequence, especially in the 10 bp PAM-adjacent sequence,⁸⁻¹⁰ so sgRNAs with high similarity to other genomic sequences should be avoided. To avoid unwanted off-target effects of sgPal in human and mouse applications, the inventors searched for 10 bp sequences largely unique to the mouse and human genomes. CRISPR is highly specific but can tolerate up to 5 nucleotide (nt) mismatches between the sgRNA and template DNA⁶. Cas9 will cleave at nonspecific sites with a low efficiency so long as no more than 2 nucleotide differences occur within the final 1 lnt, and crucially a PAM sequence must be present at the 3 bp directly downstream of the complementary region.^{10,11} sgPal sequence similarity to off-target genomic loci was determined by BLAST comparison of the 10 bp mirrored sequences to the mouse and human genomes.

Table 2 lists loci with 2 or fewer mismatches between the final 11nt of the palindromic sequence and the mouse and human genomes for all four palindromic sgRNAs described herein.

TABLE 2
Predicted off-target effects of scCRISPR palindromic sgRNAs

| | | | Missmatches | | |
|---|---|---|---|---|---|
| Name | Sequence | BLAST hits | Overall | Last 11nt | 3′ nt |
| sgPal1 | GCTCTGTGACT AGTCACAGAG | | | | . . . |
| | | | | | . . . |
| sgPal2 | GCGGAACACA TGTGTTCCG | | | | . . . |
| | | | | | . . . |
| | | | | | . . . |
| | | | | | . . . |
| | | | | | . . . |
| | | | | | . . . |
| sgPal3 | TCGATCGTCG CGACGATCGA | Human Chr. 16 | 5 | 1 | . . . GGC |
| | | Human Chr. 22 | 6 | 2 | . . . GGT |
| | | Mouse Chr. 11 | 6 | 1 | . . . AGC |
| | | Mouse Chr. 2 | 7 | 2 | . . . GGC |
| sgPal4 | CGACGATCGA TCGATCGTCG | Human Chr. 17 | 6 | 1 | . . . GAA |
| | | Human Chr. 11 | 6 | 2 | . . . AAA |
| | | Mouse Chr. 5 | 4 | 0 | . . . TTG |
| | | Mouse Chr. 5 | 6 | 2 | . . . GTG |
| | | Mouse Chr. 5 | 7 | 2 | . . . AAG |
| | | Mouse Chr. 15 | 5 | 1 | . . . TTC |
| | | Mouse Chr. 18 | 4 | 1 | . . . TCC |
| | | Mouse Chr. 19 | 5 | 1 | . . . GGT |

The sgPal sequences chosen have too many dissimilarities with the mouse and human genomes for these sites to be recognized as binding sites. In all cases, sites sharing some similarity with the palindromic sequences lack an “-NGG” sequence following immediately downstream. The current understanding of determinants of CRISPR-specificity predict that the sgPal sequences should not induce off-target cleavage.

scCRISPR

The inventors obtained four sets of oligonucleotides to clone palindromic sgRNA targeting sequences for use in scCRISPR (Table 2). scCRISPR palindromic sgRNAs comprise an initial ‘G’ nucleotide followed by an 18 or 20 bp palindromic sequence. A published cloning protocol was used to clone these sequences into a BbsI-digested plasmid subcloned from the pX330 sgRNA expression cassette into a plasmid with a pT2AL200R175 backbone¹², Hygromycin resistance⁵, and with a modified hairpin structure to incorporate the “FE” alterations shown to improve guide RNA hairpin stability¹³. Because the two nucleotides at the end of the U6 promoter immediately upstream of the sgRNA targeting sequence are ‘CC,’ the cloned palindromic targeting sequence of the sgRNA is of the form ‘CCG[18-20 bp palindromic sgRNA sequence].’ The reverse complement of this sequence is ‘[18-20 bp palindromic sgRNA sequence]CGG,’ so palindromic sgRNAs of this form are capable of self-cleaving once they are transcribed in target cells and complex with Cas9. The CBh Cas9 expression cassette from pX330 was also subcloned into a plasmid with a pT2AL200R175 backbone¹² and Blasticidin resistance.

To prepare site-specific sgRNA homology fragments, a two-step PCR amplification protocol was designed. An oligonucleotide was obtained from Integrated DNA Technologies (IDT) that contains the sgRNA sequence and ˜20 bp of homology to the upstream and downstream regions of the sgRNA expression cassette. All specific oligonucleotides are in Table 3 and are of the form:

For 20 bp sgRNA
TGGAAAGGACGAAACACCGN19GTTTAAGAGCTATGCTGGAAAC
For 21 bp sgRNA
GGAAAGGACGAAACACCGN20GTTTAAGAGCTATGCTGGAAAC
For 19 bp sgRNA
TGGAAAGGACGAAACACCGN18GTTTAAGAGCTATGCTGGAAACA

25 cycles of Onetaq PCR were performed using a three-step protocol (94 degrees for 15 seconds followed by 60 degrees for 30 seconds followed by 68 degrees for 30 seconds) using the following reaction mix to add the first half of the homology arms to the sgRNA oligonucleotide:
2×Onetaq master mix with standard buffer (NEB): 50% of reaction volume
20 uM sg[LocusX]: 2.5% of reaction volume 20 uM scCRISPR_homology_fw: 2.5% of reaction volume
20 uM scCRISPR_homology_rv: 2.5% of reaction volume
dH2O: 42.5% of reaction volume

For each electroporation to be performed, at least 10 uL of reaction volume were used for this first PCR. A second PCR was then performed using the first PCR reaction as the template without purification. The primers used in this second PCR are the same for every scCRISPR amplification and add an extra 62 bp of sgRNA plasmid homology to each end for a total of ˜120 bp of sgRNA homology on each end. For this PCR, 35 cycles of Onetaq PCR were performed using a three-step protocol (94 degrees for 15 seconds followed by 60 degrees for 30 seconds followed by 68 degrees for 30 seconds) using the following reaction mix:
2×Onetaq master mix with standard buffer: 50% of reaction volume
Unpurified first PCR product: 5% of reaction volume
20 uM scCRISPR_homology_extension_fw: 2.5% of reaction volume
20 uM scCRISPR_homology_extension_rv: 2.5% of reaction volume
dH2O: 40% of reaction volume

A reaction volume of 100 uL per electroporation to be performed was used. A 2 uL aliquot of this second PCR product was run on 2% agarose to test for the expected ˜260 bp product shown below with different forms of underline to denote the initial oligonucleotide (double underline), first homology primers (single underline), and second homology primers (squiggly underline):

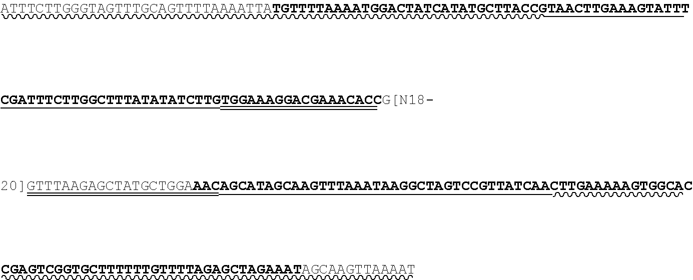

Once verified, minElute PCR purification (Qiagen) was performed on the product, loading a maximum of 200 uL of PCR product into a single minElute column.

For targeting of mESCs: the inventors then electroporated a mixture of 5 ug of CBh Cas9-BlastR plasmid, 5 ug of sgPal plasmid, and minElute purified product of 100 uL sg[LocusX] homology fragment into ˜106 mouse embryonic stem cells. For control experiments using sgRNA plasmid, a mixture of 5 ug of CBh Cas9-BlastR plasmid and 5 ug of sgLocusX plasmid were used. The DNA mixture was vacuum centrifuged to a final volume of <20 uL and 120 uL EmbryoMax Electroporation Buffer (ES-003-D, Millipore) was added to the mESCs. DNA mixture and mESC suspension were mixed and electroporated in a 0.4 cm electroporation cuvette using a BioRad electroporator at 230 V, 0.500 uF, and maximum resistance.

Electroporated cells were plated onto a single well of a 12-well tissue culture plate (BD Falcon) in >2 mL mES media supplemented with 7.5 uM Y-27632 (Tocris). From 24-72 hours after electroporation, media was refreshed daily with mES media supplemented with 10 ug/mL Blasticidin (Life Technologies) and 66 ug/mL (1:666) Hygromycin (CellGro). After selection, media was refreshed every day and cells were trypsinized and replated when confluent. Testing of CRISPR mutation or homologous recombination efficiency was performed 7 days after electroporation.

The inventors have found that transfection using Lipofectamine 3000 (Life Technologies) using the standard protocol is slightly less effective (˜80-90% as efficient) than electroporation at scCRISPR and Linear-CRISPR in mESCs. For 293FT experiments, the inventors used Lipofectamine transfection, as this cell line is known to be particularly amenable to transfection. For targeting of hESCs: a mixture of 5 ug of CBh Cas9-BlastR plasmid, 5 ug of sgPal plasmid, and minElute purified product of 100 uL sg[LocusX] homology fragment was electroporated into ˜106 human embryonic stem cells depleted of feeder cells. For control experiments using sgRNA plasmid, a mixture of 5 ug of CBh Cas9-BlastR plasmid and 5 ug of sgLocusX plasmid were used. The DNA mixture was vacuum centrifuged to a final volume of <20 uL and 100 uL electroporation buffer from the Amaxa Human Stem Cell Nucleofector kit 1 was added to the hESCs. DNA mixture and hESC suspension were mixed and electroporated in an Amaxa Nucleofector II with program B-16.

Electroporated cells were plated onto a single well of a 6-well tissue culture plate (BD Falcon) previously coated with gelatin and irradiated MEFs in >2 mLcomplete hES media supplemented with 10 uM Y-27632 (Tocris). From 24-72 hours after electroporation, media was refreshed daily with complete hES media supplemented with 2 ug/mL Blasticidin (Life Technologies) and 66 ug/mL (1:666) Hygromycin (CellGro). After selection, media was refreshed every day and cells were trypsinized and replated when confluent. Testing of CRISPR mutation or homologous recombination efficiency was performed at the first and second passages, circa 10 and 14 days after electroporation.

### Linear-CRISPR

gBlock sequences containing the full U6 promoter, locus-specific sgRNA, and FE-modified gRNA hairpin were ordered from IDT as gBlocks using the following template:

GAGTATTACGGCATGTGAGGGCCTATTTCCCATGATTCCTTCATATTTG
CATATACGATACAAGGCTGTTAGAGAGATAATTGGAATTAATTTGACTG
TAAACACAAAGATATTAGTACAAAATACGTGACGTAGAAAGTAATAATT
TCTTGGGTAGTTTGCAGTTTTAAAATTATGTTTTAAAATGGACTATCAT
ATGCTTACCGTAACTTGAAAGTATTTCGATTTCTTGGCTTTATATATCT
TGTGGAAAGGACGAAACACCG
[N18-20]
GTTTAAGAGCTATGCTGGAAACAGCATAGCAAGTTTAAATAAGGCTAGT
CCGTTATCAACTTGAAAAAGTGGCACCGAGTCGGTGCTTTTTTGTTTTA
GAGCTAGAAATAGCAAGTTAAAATAAGGCTAGTCCGTTTTTAGCGCGTG
CGCCAATTCTGCAGACAAATGGCTCTAGAGGTACGGCCGCTTCGAGCAG
ACATGATAAGATACATTGA

For 21 bp sgRNAs, the final A was omitted, and for 19 bp sgRNAs, a T was added at the beginning.

35 cycles of Onetaq PCR amplification were then performed on the gBlock using a three-step protocol (94 degrees for 15 seconds followed by 60 degrees for 30 seconds followed by 68 degrees for 30 seconds) using the following reaction mix:
2× Onetaq master mix with standard buffer: 50% of reaction volume
gBlock resuspended at 1 ng/uL: 0.25% of reaction volume
20 uM Linear-CRISPR_fw: 2.5% of reaction volume
20 uM Linear-CRISPR_rv: 2.5% of reaction volume
dH2O: 44.75% of reaction volume

A reaction volume of 100 uL per electroporation to be performed was used. A 2 uL aliquot of this PCR product was run on 2% agarose to test for the expected 500 bp product. Once verified, minElute PCR purification (Qiagen) was performed on the product, loading a maximum of 200 uL of PCR product into a single minElute column. Alternatively, equivalent results were acheived when existing sgRNA plasmids were PCR-amplified with the same Linear-CRISPR fw and rv primers which also occur in this sgRNA plasmid.

For targeting of mESCs: a mixture of 5 ug of CBh Cas9-BlastR plasmid and minElute purified product of 100 uL sg[LocusX] gBlock fragment was electroporated into ˜106 mouse embryonic stem cells using the same protocol as above. Electroporated cells were plated onto a single well of a 12-well tissue culture plate (BD Falcon) in >2 mL mES media supplemented with 7.5 uM Y-27632 (Tocris). From 24-72 hours after electroporation, media was refreshed daily with mES media supplemented with 10 ug/mL Blasticidin (Life Technologies) only since no Hygromycin plasmid was added. After selection, media was refreshed every day and cells were trypsinized and replated when confluent. Testing of CRISPR mutation or homologous recombination efficiency was performed 7 days after electroporation.

For targeting of hESCs: a mixture of 5 ug of CBh Cas9-BlastR plasmid and minElute purified product of 100 uL sg[LocusX] gBlock fragment was electroporated into ˜106 human embryonic stem cells depleted of feeder cells using the same protocol as above. Electroporated cells were plated onto a single well of a 6-well tissue culture plate (BD Falcon) previously coated with gelatin and irradiated MEFs in >2 mL complete hES media supplemented with 10 uM Y-27632 (Tocris). From 24-72 hours after electroporation, media was refreshed daily with complete hES media supplemented with 2 ug/mL Blasticidin (Life Technologies). After selection, media was refreshed every day and cells were trypsinized and replated when confluent. Testing of CRISPR mutation or homologous recombination efficiency was performed at the first and second passages, circa 10 and 14 days after electroporation.

### Homologous Recombination

GFP was amplified using two successive PCR reactions to add ˜70-80 bp homology arms to each side. Homology arms were designed to encode GFP in-frame immediately upstream of the stop codon of the Hist1h3a and Nanog genes and to include a stop codon after the GFP ORF. sgRNA sequences were designed to cleave DNA as close as possible to the endogenous stop codon of the gene to be targeted. Homology arms were designed so as not to overlap with the sgRNA sequence by more than the 10 bp on the side opposite the PAM sequence and no overlap was ever allowed on the PAM side to avoid CRISPR cleavage of the GFP homology template. The first homology primer pair is of the following format:

LocusX_GFPhomologyarm_fw [LocusX pre-stop40bp]GTG
AGCAAGGGCGAGGAGCT
LocusX_GFPhomologyarm_rv [LocusX post-stop reverse
complement40bp]TGAGGAGTGAATTGCGGCCG

The common 20 bp sequences allow amplification of the entire GFP ORF and include the stop codon. These primers produce an 819 bp product. The inventors PCR amplified GFP using 35 cycles of Phusion (NEB) PCR amplification using a two-step protocol (98 degrees for 10 seconds followed by 72 degrees for 45 seconds) using the following reaction mix:
2× Phusion master mix with standard buffer: 50% of reaction volume
GFP plasmid at 100 ng/uL: 0.5% of reaction volume
20 uM LocusX_GFPhomologyarm_fw: 2.5% of reaction volume
20 uM LocusX_GFPhomologyarm_rv: 2.5% of reaction volume
DMSO: 3% of reaction volume
dH2O: 41.5% of reaction volume

For each electroporation to be performed, at least 10 uL of reaction volume was used for this first PCR. A second PCR was performed using the first PCR reaction as the template without purification. For this PCR, 60 bp primers that extend the locus-specific homology by 30-40 bp on each end were used. To do so, the inventors designed a set of PCR primers that overlapped with the first homology arm by 20-30 bp. The inventors chose the minimal overlap such that the overlapping region was estimated to have a Tm of >65 degrees using the NEB Tm calculator. The unpurified product of the previous reaction was then PCR amplified using 35 cycles of Phusion PCR amplification using a two-step protocol (98 degrees for 10 seconds followed by 72 degrees for 45 seconds) using the following reaction mix:
2× Phusion master mix with standard buffer: 50% of reaction volume
Unpurified product of PCR1: 5% of reaction volume
20 uM LocusX_homologyarmextension_fw: 2.5% of reaction volume
20 uM LocusX_homologyarmextension_rv: 2.5% of reaction volume
DMSO: 3% of reaction volume
dH2O: 37% of reaction volume

For each electroporation to be performed, at least 100 uL of reaction volume was used for this second PCR. A 2 uL aliquot of this PCR product was run on 2% agarose to test for the expected ˜900 bp product. Once verified, minElute PCR purification (Qiagen) was performed on the PCR product, loading a maximum of 200 uL of PCR product into a single minElute column.

For targeting mESCs: the invenotrs then electroporated a mixture of 5 ug of CBh Cas9-BlastR plasmid, minElute purified product of 100 uL GFP LocusX homology arm fragment, and either gBlock or sgPal and homology fragment at the same amounts as mentioned above into ˜106 mouse embryonic stem cells using the same protocol as above. Electroporated cells were plated onto a single well of a 12-well tissue culture plate (BD Falcon) in >2 mL mES media supplemented with 7.5 uM Y-27632 (Tocris). From 24-72 hours after electroporation, media was refreshed daily with mES media supplemented with 10 ug/mL Blasticidin and 66 ug/mL (1:666) Hygromycin (only with sgPal, not with gBlock). After selection, media was refreshed every day and cells were trypsinized and replated when confluent. Testing of homologous recombination efficiency was performed 7 days after electroporation.

For hESCs: the inventors electroporated a mixture of 5 ug of CBh Cas9-BlastR plasmid, minElute purified product of 100 uL GFP LocusX homology arm fragment, and either gBlock or sgPal and homology fragment at the same amounts as mentioned above into ˜106 human embryonic stem cells depleted of feeder cells using the same protocol as above. Electroporated cells were plated onto a single well of a 6-well tissue culture plate (BD Falcon) previously coated with gelatin and irradiated MEFs in >2 mLcomplete hES media supplemented with 10 uM Y-27632 (Tocris). From 24-72 hours after electroporation, media was refreshed daily with complete hES media supplemented with 2 ug/mL Blasticidin and 66 ug/mL (1:666) Hygromycin (only with sgPal, not with gBlock). After selection, media was refreshed every day and cells were trypsinized and replated when confluent. Testing of CRISPR mutation or homologous recombination efficiency was performed at the first and second passages, circa 10 and 14 days after electroporation.

An example of the homologous recombination PCR strategy is shown below for the Hist1h3a locus. The 20 bp sgRNA sequence is in bold with the PAM sequence in green. The endogenous stop codon is in yellow. The homology arm primers are underlined with the first homology region in underline, the region shared between the first and second homology region in underline, and the second homology region in underline. Also included is the upstream PCR primer used to verify presence of the fusion protein, which is in red. All primer sequences are shown in Table 3. The result of this homology recombination is in-frame GFP integration immediately before the endogenous stop codon.

CCTTGTGGGTCTGTTTGAGGACACCAACCTGTGCGCCATCCACGCCAAGCGTGTCACCAT

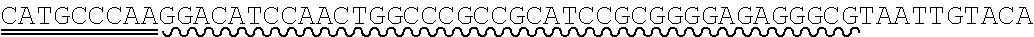

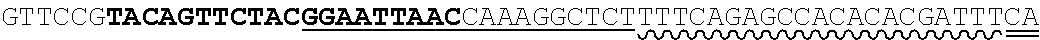

AGTAAAATGGCTGTAATTTACTCCATCCTTAAACGAA

TABLE 3
Oligonucleotides used in Examples 1 & 2
Palindromic sgRNA sequences

| | | |
|---|---|---|
| sgPal1 | GCTCTGTGACTAGTCACAGAG | Most efficient |
| | | version tested, |
| | | used for the |
| | | majority of |
| | | experiments |
| sgPal2 | GCGGAACACATGTGTTCCG | |
| sgPal3 | GTCGATCGTCGCGACGATCGA | |
| sgPal4 | GCGACGATCGATCGATCGTCG | |

sgRNA sequences for plasmid control tests

| | |
|---|---|
| sgGFP3 | GCTGAAGCACTGCACGCCGT |
| sgHist1h3a | GTTAATTCCGTAGAACTGTA |
| sgNanog | GTATGAGACTTACGCAACATC |

scCRISPR primer and sgRNA sequences

| | | |
|---|---|---|
| scCRISPR_homology_fw | TAACTTGAAAGTATTTCGATTTCTTGGCTTTATATATCTTGTGGAAAGG | Used in first PCR |
| | ACGAAACACCG | of all scCRISPR |
| | | oligos |
| scCRISPR_homology_rv | GTTGATAACGGACTAGCCTTATTTAAACTTGCTATGCTGTTTCCAGCAT | Used in first PCR |
| | AGCTCTTAAAC | of all scCRISPR |
| | | oligos |
| scCRISPR_homology_extension_fw | ATTTCTTGGGTAGTTTGCAGTTTTAAAATTATGTTTTAAAATGGACTAT | Used in second PCR |
| | CATATGCTTACCGTAACTTGAAAGTATTTCGATTTCTTGGC | of all scCRISPR |
| | | oligos |
| scCRISPR_homology_extension_rv | ATTTTAACTTGCTATTTCTAGCTCTAAAACAAAAAAGCACCGACTCGGT | Used in second PCR |
| | GCCACTTTTTCAAGTTGATAACGGACTAGCCTTATTTAAAC | of all scCRISPR |
| | | oligos |
| sgGFP1 | GGGCGAGGAGCTGTTCACCG | All scCRISPR |
| | | oligonucleotides |
| | | were ordered using |
| | | format described in |
| | | the methods |
| sgGFP2 | GAGCTGGACGGCGACGTAAA | |
| sgGFP3 | GCTGAAGCACTGCACGCCGT | |
| sgHist1h3a | GTTAATTCCGTAGAACTGTA | |
| sgNanog | GTATGAGACTTACGCAACATC | |
| sgDsRed1 | GAACTCCTTGATGACGTCCT | |
| sgDsRed2 | GCCAAGCTGAAGGTGACCAA | |
| sgH2BJ | GCGCTAAGTAAACAGTGAGT | |
| sgEsrrb | GTGATGGCCCAGCACATGGA | |
| sgFam25c | GGCCAGCCATGCTGGTAGGC | |
| sgGata6 | GCCTGAGCTGGTGCTACCAAG | |
| sgKlf4 | GCACTTTTAAATCCCACGTAG | |
| sgNfya | GTTTCCTAACCACAGGAGGG | |
| sgRpp25 | GCTCAGAGGCGAGAATTCTC | |
| sgSox2 | GTGAGGGCTGGACTGCGAAC | |
| sgTdgf1 | GAGATGGGGTACTTCTCATCC | |
| sgZfp42 | GAATGAACAAATGAAGAAAA | |

Linear-CRISPR

| | | |
|---|---|---|
| Linear-CRISPR_fw | TGAGTATTACGGCATGTGAGGGC | Used in PCR of all |
| | | Linear-CRISPR |
| | | gBlocks |
| Linear-CRISPR_rv | TCAATGTATCTTATCATGTCTGCTCGA | Used in PCR of all |
| | | Linear-CRISPR |
| | | gBlocks |

Homologous recombination primers

| | |
|---|---|
| Hist1h3aHDR_GFP_fw | GGACATCCAACTGGCCCGCCGCATCCGCGGGGAGAGGGCG |
| | GTGAGCAAGGGCGAGGAGCT |
| Hist1h3aHDR_GFP_rv | AAATCGTGTGTGGCTCTGAAAAGAGCCTTTGGTTAATTCC |
| | TGAGGAGTGAATTGCGGCCG |
| Hist1h3aHDR_Extension_fw | TGTGCGCCATCCACGCCAAGCGTGTCACCATCATGCCCAAGGACATCC |
| | AACTGGCCCGCC |
| Hist1h3aHDR_Extension_rv | TTCGTTTAAGGATGGAGTAAATTACAGCCATTTTACTTGAAATCGTGT |
| | GTGGCTCTGAAA |
| NanogHDR_GFP_fw | ATTATTCCTGAACTACTCTGTGACTCCACCAGGTGAAATA |
| | GTGAGCAAGGGCGAGGAGCT |
| NanogHDR_GFP_rv | GAAGGAACCTGGCTTTGCCCTGACTTTAAGCCCAGATGTT |
| | TGAGGAGTGAATTGCGGCCG |
| NanogHDR_Extension_f | CCATGCGCATTTTAGCACCCCACAAGCCTTGGAATTATTCCTGAACTA |
| NanogHDR_Extension_rv | aataaatctttaaaaaaaaTATGAAAATATTTGGAAGAAGGAAGGAACCTG |
| | GCTTTGCCC |
| H2BJHDR_GFP_fw | CGAGGGTACTAAGGCCGTCACCAAGTACACCAGCGCTAAG |
| | GTGAGCAAGGGCGAGGAGCT |
| H2BJHDR_GFP_rv | GGTGGCTCTTAAAAGAGCCGTTAGGGTTGAGAGTTTGCAA |
| | TGAGGAGTGAATTGCGGCCG |
| H2BJHDR_Extension_fw | CCTGCTGCTGCCTGGGGAGTTGGCCAAGCACGCCGTGTCCGAGGGTA |
| | CTAAGGCCGTCAC |
| H2BJHDR_Extension_rv | AGGAGGAATACAAGCACCAGCTCTTTCTTTGAGAACATGGGTGGCTC |
| | TTAAAAGAGCCGT |
| Esrrb_GFP_fw | CATGCACAAACTCTTCCTGGAGATGCTGGAGGCCAAGGTGGTGAGCA |
| | AGGGCGAGGAGCT |
| Esrrb_GFP_rv | CGAGGCTGGTGGCTGTGGAGGTCTCCACTTGGATCGTGTC |
| | TGAGGAGTGAATTGCGGCCG |
| Esrrb_Extension_fw | ACACTTCTACAGTGTGAAACTGCAGGGCAAGGTGCCCATGCACAAAC |
| | TCTTCCTGGAGAT |
| Esrrb_Extension_rv | CTGGGACAGCTCAGAGCCCCGATGCGGGTGTGAAAAAAGTCGAGGC |
| | TGGTGGCTGTGGAG |
| Fam25c_GFP_fw | TGTTACCCATGCGGCAGAAGGCCTGGGAAGACTGGGACAG |
| | GTGAGCAAGGGCGAGGAGCT |
| Fam25c_GFP_rv | TCACGTTTCACACTCTTTATTGACCTTCAGGAAGGGCCAG |
| | TGAGGAGTGAATTGCGGCCG |
| Fam25c_Extension_fw | AGGAGGTCACTGAGAAGGTCACCCACACCATCACTGATGCTGTTACC |
| | CATGCGGCAGAAG |
| Fam25c_Extension_rv | ATTCCATCCAAACAGAGGTAAACTCAGGACTCTGTTCACGTTTCACAC |
| | TCTTTATTGACC |
| Gata6_GFP_fw | CTCCGTGCGACAGGATTCTTGGTGTGCTCTGGCCCTGGCC |
| | GTGAGCAAGGGCGAGGAGCT |
| Gata6_GFP_rv | AATATCAGACACAAGTGGTATGAGGCCTTCAGAGCCCTCC |
| | TGAGGAGTGAATTGCGGCCG |
| Gata6_Extension_fw | CATAGGTGTCAGTCTGTCCTCCCCTGCCGAAGTCACATCCTCCGTGCG |
| | ACAGGATTCTTG |
| Gata6_Extension_rv | GTCTGCATTTTTGCTGCCATCTGGACTGCTGGACAATATCAGACACAA |
| | GTGGTATGAGGC |
| Klf4_GFP_fw | CAGGTCGGACCACCTTGCCTTACACATGAAGAGGCACTTTGTGAGCA |
| | AGGGCGAGGAGCT |
| Klf4_GFP_rv | AAAAAAAATACTGAACTCTCTCTCCTGGCAGTGTGGGTCA |
| | TGAGGAGTGAATTGCGGCCG |
| Klf4_Extension_fw | ACCGGCCCTTTCAGTGCCAGAAGTGTGACAGGGCCTTTTCCAGGTCG |
| | GACCACCTTGCCT |
| Klf4_Extension_rv | TCCCCTCGTGGGAAGACAGTGTGAAAGGTTAGAAAAAAAAATACTGA |
| | ACTCTCTCTCCTG |
| Nfya_GFP_fw | AGCTGACGAAGAAGCCATGACACAGATCATCCGAGTTTCCGTGAGCA |
| | AGGGCGAGGAGCT |
| Nfya_GFP_rv | CCATTTCCAGAACAGTGGAGAGGACCGTGACTGATCAGCT |
| | TGAGGAGTGAATTGCGGCCG |
| Nfya_Extension_fw | AGGACTGTTGTGCTGTCTCTCTCTGTAGGATCCAAACCAAGCTGACGA |
| | AGAAGCCATGAC |
| Nfya_Extension_rv | AGTGAGACTGTCAGTGCCCCACTGGAAGTCAGTCCATTTCCAGAACA |
| | GTGGAGAGGACCG |
| Rpp25_GFP_fw | TCAGCCTGAGCCAGAGGCTGAGAATGAGGACAGGACCGCC |
| | GTGAGCAAGGGCGAGGAGCT |
| Rpp25_GFP_rv | GTGTTGAAGATATATGATTCAGTCGGTCTGGGTGGCTCAG |
| | TGAGGAGTGAATTGCGGCCG |
| Rpp25_Extension_fw | TGGGGGAATCTGCTGCTGAAGAAGGCACCGCTAAGCGGTCTCAGCCT |
| | GAGCCAGAGGCTG |
| Rpp25_Extension_rv | TATGAAAGGTGCGTGTGTTGAAAGGTATGCAGGAGTGTTGAAGATAT |
| | ATGATTCAGTCGG |
| Sox2_GFP_fw | CGGCACGGCCATTAACGGCACACTGCCCCTGTCGCACATG |
| | GTGAGCAAGGGCGAGGAGCT |
| Sox2_GFP_rv | CCTCCCAATTCCCTTGTATCTCTTTGAAAATCTCTCCCCT |
| | TGAGGAGTGAATTGCGGCCG |
| Sox2_Extension_fw | GACTGCACATGGCCCAGCACTACCAGAGCGGCCCGGTGCCCGGCACG |
| | GCCATTAACGGCA |
| Sox2_Extension_rv | ATTATCAGATTTTTCCTACTCTCCTCTTTTTGCACCCCTCCCAATTCCCTT |
| | GTATCTCTT |
| Tdgf1_GFP_fw | TTGTCTTTTCCTCCAACGTTTTTACGAGCCGTCGAAGATG |
| | GCTAGCAAAGGAGAAGAACT |
| Tdgf1_GFP_rv | AAGTGGCTATCTCCAGCAACCAAAAAGTCAAGGTTA |
| | TCGCGATTTTACCACATTTGTAGA |
| Tdgf1_Extension_fw | TGGCTTTATGAACTAAAGCCATCTGCTAATATTGTGTTTCTTGTCTTTT |
| | CCTCCAACGTT |
| Tdgf1_Extension_rv | GCAAGACAAAAATCAGAGCGTCATAGAACGTGATTTTCCGAAGTGGC |
| | TATCTCCAGCAAC |
| Zfp42_GFP_fw | AGGAAGCAGCTAAGACAACATGAATGAACAAAAAATGAAT |
| | GTGAGCAAGGGCGAGGAGCT |
| Zfp42_GFP_rv | GGGCTCTTCCGCCCGGCCCTTTCTGGCCACTTGTCT |
| | TCGCGATTTTACCACATTTGTAGA |
| Zfp42_Extension_fw | GATCAGTGCCCCCTGGAAGTGAGTCATAGGCATTGTTCAAGAAGGAA |
| | GCAGCTAAGACAA |
| Zfp42_Extension_rv | ACTGGCCTTGCCTCGTCTTGCTTTAGGGTCAGTCTGTCGAGGGCTCTT |
| | CCGCCCGGCCCT |

Primers for sequencing

| | |
|---|---|
| Hist3.1-GFP_fw | CCTTGTGGGTCTGTTTGAGGA |
| GFP_rv | GTCTTTGCTCAGGGCGGACT |

Flow Cytometry

Cells to be analyzed by flow cytometry were trypsinized, quenched, and fluorescence of 2×10⁴ cells was measured using a BD Accuri C6 flow cytometer and accompanying software (BD Biosciences).

Fluorescence Imaging

Live cell imaging was performed using a DMI 6000b inverted fluorescence microscope (Leica), and image analysis with the Leica AF6000 software package.

### REFERENCES

1. Sherwood R I, Hashimoto T, O'Donnell C W, et al. Discovery of directional and nondirectional pioneer transcription factors by modeling DNase profile magnitude and shape. Nat Biotechnol. February 2014; 32(2):171-178.
2. Cong L, Ran F A, Cox D, et al. Multiplex genome engineering using CRISPR/Cas systems. Science. February 2013; 339(6121):819-823.
3. Mali P, Yang L, Esvelt K M, et al. RNA-guided human genome engineering via Cas9. Science February 2013; 339(6121):823-826.
4. Fu Y, Sander J D, Reyon D, Cascio V M, Joung J K. Improving CRISPR-Cas nuclease specificity using truncated guide RNAs. Nat Biotechnol. March 2014; 32(3):279-284.
5. Ran F A, Hsu P D, Wright J, Agarwala V, Scott D A, Zhang F. Genome engineering using the CRISPRCas9 system. Nat Protoc. November 2013; 8(11):2281-2308.
6. Cho S W, Kim S, Kim Y, et al. Analysis of off-target effects of CRISPR/Cas-derived RNA-guided endonucleases and nickases. Genome Res. January 2014; 24(1): 132-141.
7. Gilbert L A, Larson M H, Morsut L, et al. CRISPR-mediated modular RNA-guided regulation of transcription in eukaryotes. Cell. July 2013; 154(2):442-451.
8. Fu Y, Foden J A, Khayter C, et al. High-frequency off-target mutagenesis induced by CRISPR-Cas nucleases in human cells. Nat Biotechnol. September 2013; 31(9):822-826.
9. Wu X, Scott D A, Kriz A J, et al. Genome-wide binding of the CRISPR endonuclease Cas9 in mammalian cells. Nat Biotechnol. July 2014; 32(7):670-676.
10. Kuscu C, Arslan S, Singh R, Thorpe J, Adli M. Genome-wide analysis reveals characteristics of off target sites bound by the Cas9 endonuclease. Nat Biotechnol. July 2014; 32(7):677-683.
11. Lin Y, Cradick T J, Brown M T, et al. CRISPR/Cas9 systems have off-target activity with insertions or deletions between target DNA and guide RNA sequences. Nucleic Acids Res. 2014; 42(11):7473-7485.
12. Urasaki A, Morvan G, Kawakami K. Functional dissection of the Tol2 transposable element identified the minimal cis-sequence and a highly repetitive sequence in the subterminal region essential for transposition. Genetics. October 2006; 174(2):639-649.
13. Chen B, Gilbert L A, Cimini B A, et al. Dynamic imaging of genomic loci in living human cells by an optimized CRISPR/Cas system. Cell. December 2013; 155(7):1479-1491.

### Example 3: Exemplary Protocols

This Example provides examples of protocols for carrying out the modified CRISPR reactions as described herein. These protocols are not intended to be limiting and can be further optimized by one of skill in the art to improve efficiency or speed of the reaction. Alternatively, the protocols described herein can be modified to use reagents other than the PCR reagents described herein. General modification or optimization of PCR protocols is well within the skill set of one of ordinary skill in the art.

CRISPR/Cas9 gene editing technology has greatly advanced genetics and molecular biology research. By design of a site-specific single guide RNA (sgRNA) that interacts with the Cas9 endonuclease, DNA cleavage can be directed to almost any genomic site of interest¹⁻³. It is the most efficient technology to mutate, delete, and insert genomic DNA sequences at specific genomic loci that has been developed to date, offering exciting opportunities to improve understanding of genome function⁴.

Site-specific genomic targeting generally requires molecular cloning of sgRNA plasmids for each novel targeted locus. This multistep process is both time-consuming and prone to missteps that delay an otherwise rapid process of genome editing. Furthermore, the cost associated with plasmid ligation, transformation, purification, and sequence verification of each newly generated sgRNA over the course of roughly one week are prohibitive to large-scale arrayed screening applications necessary for high-throughput genome editing platforms.⁵

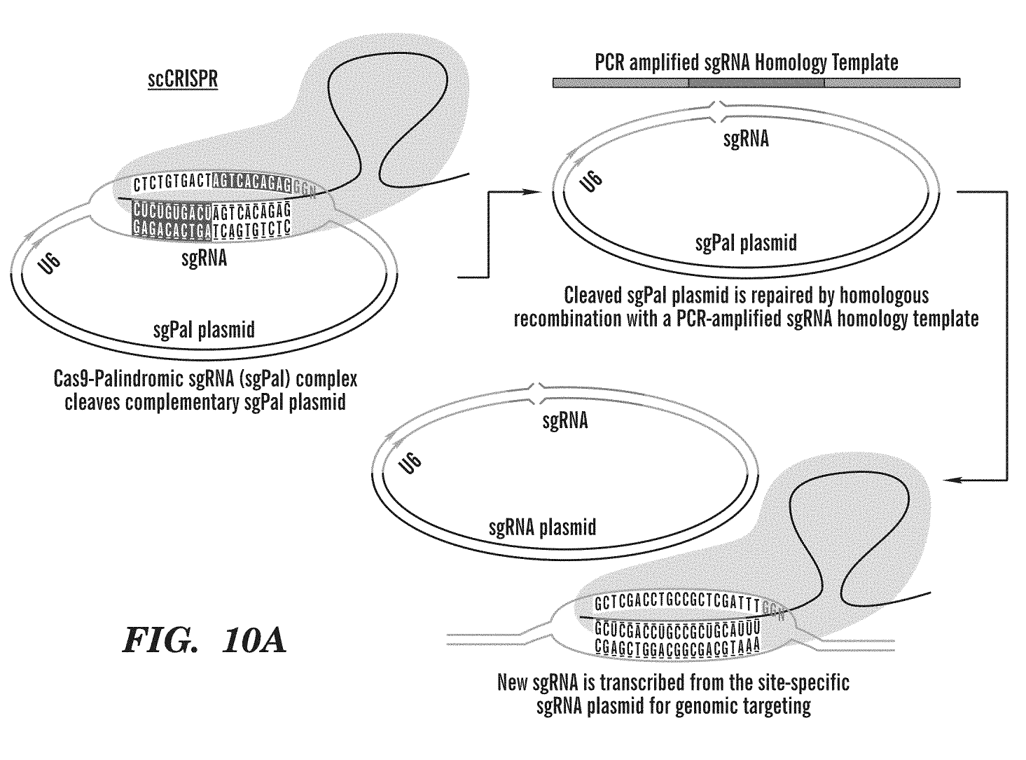

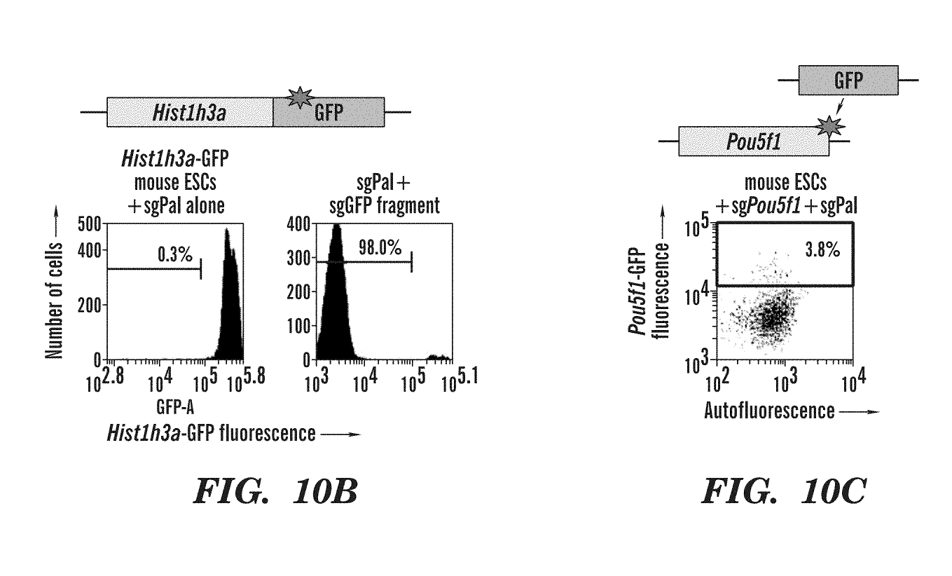

The methods provided herein overcome this barrier by the development of a self-cleaving palindromic sgRNA plasmid (sgPal) that targets and cuts its own sgRNA sequence once expressed in cells in the presence of Cas9 (FIG. 10A)⁴. The methods provided herein harness, for example, the ability of embryonic stem cells (ESCs) and cancer cell lines to repair double-stranded breaks by homologous recombination to generate a new site-specific genome-targeting sgRNA from a short, PCR amplified sgRNA template within target cells. By self-cloning CRISPR (scCRISPR), cells can be targeted within two hours after sgRNA oligos are obtained, without compromising on mutational targeting efficiency while saving significant time, effort, and cost.

For the generation of knock-in cell lines, additional constructs are traditionally made with long homology regions to facilitate insertion of the desired sequence at the locus of interest. Typically, flanking homology arms range from 600-6000 bp long each, and must be cloned on either side of the insertion sequence to generate a targeting plasmid. Obtaining large genomic sequences from the genome by PCR amplification frequently requires multiple rounds of amplification, ligation and cloning, spanning weeks, and is often unsuccessful. With recent advances in oligonucleotide synthesis, these sequences can be bought, though at significant expense. Either method of homology-plasmid construction becomes a substantial investment to be made for each new insert and every knock-in site.

As such, CRISPR gene editing is currently under-utilized for generating reporter cell models. It is demonstrated in this study that short, ±80 bp flanking homology sequences are sufficient and highly effective at knock-in of ±2 kb long sequences. The necessary homology arms are easily extended from the insert sequence by PCR amplification, in a rapid protocol spanning less than 2 hours. As such, knock-in cell targeting with scCRISPR is done within a day, and knock-in lines can be generated and verified in 2-3 weeks, depending on the knock-in sequence and genomic locus. The cloning-free methods of CRISPR/Cas9 gene editing described herein considerably lower the bar on genetic engineering and transgenesis of in vitro cell models, as far as cost, time, and effort, without compromising targeting efficiency. Thus, scCRISPR as an ideal technique for inducing individual gene modifications, and large-scale and high-throughput gene editing applications alike.

Note:

Cells for scCRISPR targeting are cultured and maintained according to standard practices in a Class II biological hazard flow hood or a laminar-flow hood.

Basic Protocol 1 scCRISPR Site Specific Targeting for Non-Homologous End-Joining

Site-specific sgRNA oligos are designed with short flanking regions homologous to the sgPal plasmid, and then further extend the homology regions by consecutive PCR amplification steps for optimal efficiency of recombination with the sgPal plasmid. Only one oligo must be designed and ordered for each desired target site, and the homology arm primers are all stock reagents. This locus-specific sgRNA PCR product is introduced into target cells along with plasmids encoding Cas9 and the self-cleaving sgPal, and cells that have received these plasmids are enriched by transient antibiotic selection. The locus-specific sgRNA recombines with sgPal inside the host cell, routinely yielding mutation in >90% of cells.

Materials
sgRNA oligos
PCR polymerase (OneTaq)
Agarose
Gel electrophoresis unit
Minelute DNA purification kit (or standard DNA purification followed by vacuum concentration)
sgPal7-HygR plasmid maxiprep DNA (Addgene #71484)
CBH-Cas9-BlastR plasmid maxiprep DNA (Addgene (#71489)
Sterile 1.5 ml microcentrifuge tubes
Cell electroporator and cuvettes
Cells and appropriate cell culture media and reagents
Y-27632 ROCK-inhibitor
Hygromycin
Blasticidin
   Step 1—scCRISPR sgRNA Preparation

1. Design sgRNAs for target locus by identifying the genomic Protospacer Adjacent Motif (PAM)-sequence ( . . . NGG) of interest. Note that spCas9 cleavage occurs between bases 3-4 upstream of the PAM: for mutation or knockout, this should be chosen to disrupt the function of interest. For gene knock-in, guidelines are given in Basic Protocol 2. The “NGG” can be on either strand, so the PAM-sequence “CCN” is also an acceptable target.

2. Once you have identified the appropriate PAM, you will design an oligonucleotide from the genomic sequence upstream of the PAM, to be ordered as the protospacer, following the guidelines outlined below. The protospacer sequence should preferably be 19-21 bp long. Because the U6 promoter, which will be used to transcribe the gRNA is most efficient when the first base of the protospacer is a ‘G’, the following scheme is used to design the oligonucleotide:

a. Protospacers preferably have 19-21 bp of homology to the genome immediately preceding the NGG “PAM” sequence:
i. If the genome sequence is GNNNNNNNNNNNNNNNNNNNNGG (GN19NGG), the protospacer sequence should be GNNNNNNNNNNNNNNNNNN (GN19)
ii. If “i” is not satisfied but GNNNNNNNNNNNNNNNNNNNGG (GN18NGG) is satisfied, the protospacer sequence should be GNNNNNNNNNNNNNNNNNN (GN18)
iii. If “i” and “ii” are not satisfied, the protospacer sequence should be GNNNNNNNNNNNNNNNNNNNN (GN20) immediately upstream of the NGG, regardless of whether the “G” at position 1 is in the genome or not.
iv. If the genomic sequence targeted is CCN(N20), then reverse complement the entire stretch and apply these rules to the reverse complemented sequence.

b. After applying these rules, you should have a protospacer sequence that is 19-21 bp long. The sgRNA oligonucleotide to order for scCRISPR order contains this 19-21 bp sequence, flanked by sgPal homology sequences that will be used to PCR amplify the protospacer into a functional homology template for the scCRISPR system. The oligonucleotide should obey the following format in Table 4:

TABLE 4
scCRISPR olig design

| | 60bp scCRISPR oligo design |
|---|---|
| For 20 bp sgRNA | TGGAAAGGACGAAACACCGN19GTTTAAGAGCTA |
| | TGCTGGAAAC |
| For 21 bp sgRNA | GGAAAGGACGAAACACCGN20GTTTAAGAGCTAT |
| | GCTGGAAAC |
| For 19 bp sgRNA | TGGAAAGGACGAAACACCGN18GTTTAAGAGCTA |
| | TGCTGGAAACA |

3. Order standard scCRISPR homology directed repair (HDR) extension primers for 3 consecutive PCR steps:

TABLE 5
Examples of extension primer sequences

| PCR | | |
|---|---|---|
| step | Primer | Sequence |
| Step 1 | sgRNA_HDRstep1_fw | TGTTTTAAAATGGACTATCATATGCTTACCGTAACTTGAAAGTATTTCGATTTCT |
| | | TGGCTTTATATATCTTGTGGAAAGGACGAAACACC |
| Step 1 | sgRNA_HDRstep1_rv | GTTGATAACGGACTAGCCTTATTTAAACTTGCTATGCTGTTTCCAGCATAGCTC |
| | | TTAAAC |
| Step 1 | sgRNA_HDRstep2_fw | GTACAAAATACGTGACGTAGAAAGTAATAATTTCTTGGGTAGTTTGCAGTTTT |
| | | AAAATTATGTTTTAAAATGGACTATCATATGCTTACC |
| Step 1 | sgRNA_HDRstep2_rv | ATTTTAACTTGCTATTTCTAGCTCTAAAACAAAAAAGCACCGACTCGGTGCCAC |
| | | TTTTTCAAGTTGATAACGGACTAGCCTTATTTAAAC |
| Step 2 | sgRNA_HDRstep3_fw | CGATACAAGGCTGTTAGAGAGATAATTAGAATTAATTTGACTGTAAACACAAA |
| | | GATATTAGTACAAAATACGTGACGTAGAAAGTAATAA |
| Step 2 | sgRNA_HDRstep3_rv | TCAATGTATCTTATCATGTCTGCTCGATTTTAACTTGCTATTTCTAGCTCTAAAA |
| | | CAAAA |
| Step 3 | sgRNA_HDRstep4_fw | GGCCTATTTCCCATGATTCCTTCATATTTGCATATACGATACAAGGCTGTTAGA |
| | | GAGATA |
| Step 3 | sgRNA_HDRstep4_rv | TCAATGTATCTTATCATGTCTGCTCGA |

Step 2—sgRNA PCR Amplification

The protocols below are described for amplification with OneTaq polymerase, but other polymerases also work for scCRISPR. sgRNA oligos are PCR amplified in three successive PCR steps for maximum recombination efficiency, and PCR purification at intermediate steps is not required. PCR steps 1 and 2 are run with 10 amplification cycles to save time, and the final PCR step 3 is run with 35 cycles to generate a large number of amplicons. Note that the initial scCRISPR protocol employed the first two PCR steps only and worked at 70-90% efficiency, but it has since been found that adding a third PCR step further increases efficiency to >90%. If targeting with the product of PCR step 2 is desired, make sure to perform 35 cycles of the final PCR step to produce sufficient amplicons for sgRNA recombination.

While further refinements or adaptations may be application, for maximal efficiency the inventors recommend the protocol as described below.

4. PCR step 1 on 60 bp sgRNA oligo template with HDRstep1 and HDRstep2 forward and reverse primers using Onetaq polymerase (Ta=60° C.), and run for 10 amplification cycles. Amplification product is 293 bp. PCR mix below is for typical 20 ul reaction volume, but can be scaled if needed.

TABLE 6
Example of amplification mixture for PCR Step 1

| | | | % Reaction |
|---|---|---|---|
| Volume | | Reagent | Volume |
| 10 | ul | OneTaq 2x Master Mix with standard buffer | 50% |
| 0.5 | ul | 20 uM stock of 60 bp sgRNA oligo | 2.5% |
| 1 | ul | 10 uM HDRstep1 Fw + Rv primer mix | 5% |
| 1 | ul | 10 uM HDRstep2 Fw + Rv primer mix | 5% |
| 7.5 | ul | mQ water | 37.5% |

5. PCR step 2 on 293 bp amplification product of PCR step 1, with HDRstep3 forward and reverse primers using Onetaq polymerase (Ta=60° C.), and run for 10 amplification cycles. The amplification product of PCR step 1 is used directly without purification, and it is unnecessary to determine DNA concentration. If desired, amplicon size can be validated by running a small aliquot of PCR step 1 on a 2% agarose by gel electrophoresis as described below in this protocol, though this is not necessary at this stage. Amplification product is 379 bp. PCR mix below is for typical 20 ul reaction volume, but can be scaled if needed.

TABLE 7
Example of amplification mixture for PCR Step 2

| | | | % Reaction |
|---|---|---|---|
| Volume | | Reagent | Volume |
| 10 | ul | OneTaq 2x Master Mix with standard buffer | 50% |
| 1 | ul | Unpurified product of PCR 1 | 5% |
| 1 | ul | 10 uM HDRstep3 Fw + Rv primer mix | 5% |
| 8 | ul | mQ water | 40% |

6. PCR step 3 on 379 bp amplification product of PCR step 2, with HDRstep3 forward and reverse primers using Onetaq polymerase (Ta=60° C.). Amplification product of PCR step 2 is used directly as described above. Amplification is done for 35 cycles, as this is the last PCR step to generate templates for scCRISPR sgPal homologous recombination. Amplification product is 415 bp. PCR mix below is for typical 100 ul reaction volume, but can be scaled if needed.

TABLE 8
Example of amplification mixture for PCR Step 3

| | | | % Reaction |
|---|---|---|---|
| Volume | | Reagent | Volume |
| 50 | ul | OneTaq 2x Master Mix with standard buffer | 50% |
| 5 | ul | Unpurified product of PCR 2 | 5% |
| 5 | ul | 10 uM HDRstep4 Fw + Rv primer mix | 5% |
| 40 | ul | mQ water | 40% |

7. Final amplicon is validated by gel electrophoresis, running a 2 ul aliquot on a 2% agarose gel. Product is as follows:

Legend: sgRNA oligo PCRstep1amplicon PCRstep2 PCRstep3

GGCCTATTTCCCATGATTCCTTCATATTTGCATATACGATACAAGGCTGTTAGAGAGA
TAATTAGAATTAATTTGACTGTAAACACAAAGATATTAGTACAAAATACGTGACGTAGA
AAGTAATAATTTCTTGGGTAGTTTGCAGTTTTAAAATTATGTTTTAAAATGGACTATCATA
TGCTTACCGTAACTTGAAAGTATTTCGATTTCTTGGCTTTATATATCTTGTGGAAAGGACG

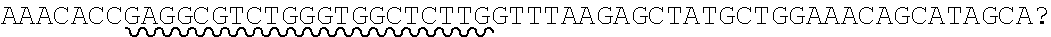

AGTTTAAATAAGGCTAGTCCGTTATCAACTTGAAAAAGTGGCACCGAGTCGGTGCTTTTT
TGTTTTAGAGCTAGAAATAGCAAGTTAAAATCGAGCAGACATGATAAGATACATTGA

8. Minelute PCR purify the remaining 98 ul of PCR step 3 in 10 ul.

Step 3—scCRISPR Cell Targeting for NHEJ

Cells can be targeted by electroporation, transfection, or nucleofection. The inventors have thus far achieved the highest efficiency cutting by electroporation, and lowest efficiency by transfection, for which the protocols follow below:

For Electroporation

9. For electroporation into 12-well, combine the 10 ul eluate of PCRstep3 from above with 4 ug CBH-Cas9-BlastR plasmid and 4 ug sgPal7-HygR plasmid. If total volume exceeds 20 ul, concentrate by vacuum centrifugation until it is reduced below this threshold: be vigilant so that the DNA mix does not get contaminated in this process and the DNA does not dry out.

10. Prepare single cell suspension by enzymatic passaging and concentrate cells by centrifugation for 5 minutes at 200 g.

11. Transfer appropriate number of cells to electroporation cuvette in 120 ul EmbryoMax Electroporation buffer and gently mix well with DNA. For mouse and human embryonic stem cells (ESCs), one can use ±10⁶ cells and 0.4 cm electroporation cuvettes.

12. Electroporate cells with appropriate settings for the cell type used. For mouse and human ESCs, the inventors typically electroporate at 230 V, 0.500 mF, and maximum resistance.

13. Plate in 12 well and incubate at 37° C. overnight. Mouse and human ESCs in complete ESC media can be plated with added 7.5 uM Y-27632 if desired to help recovery and reduce cell death following electroporation. Continue with step 15.

For Transfection

14. For transfection into 12-well, combine the 10 ul eluate of PCRstep3 from above with 0.5 ug CBHCas9-BlastR plasmid and 0.5 ug sgPal7-HygR plasmid and transfect according to the standard protocol for transfection reagent and cell type used. Incubate cells overnight at 37° C.

15. Select with Blasticidin and Hygromycin from hours 24-72 after targeting. Mouse ESCs were selected with 10 ug/mL Blasticidin+100 ug/mL Hygromycin. Human ESCs were selected with 2 ug/mL Blasticidin+66 ug/mL Hygromycin. Expect >90% cutting efficiency for electroporation and ˜50% for transfection. Representative data for knock-out efficiency is shown in FIG. 10B, by knock-out of Hist1h3a-GFP fluorescence.

Basic Protocol 2 scCRISPR Gene Insertion by Homologous Recombination

The design of an scCRISPR sgRNA oligo is described above for cutting at the genomic insertion site. In addition, ±150 bp long sequences are designed to be homologous to the genomic locus to flank the sequence that is to be inserted, such as GFP for the generation of a fluorescent reporter. It has been found that introducing GFP with 75-80 bp homology arms is sufficient to induce knock-in at appreciable frequency. Because of cost and PCR efficiency, these homology arms are added using two rounds of PCR, each of which adds 35-40 bp of homology arm to GFP. Due to cost considerations for oligonucleotide production and PCR efficiency considerations, each primer will be 60 bp at maximum.

Significant overlap between the sgRNA and genomic homology arm sequence should be avoided to prevent cleavage of the insertion cassette. Thus, to avert further disruption of the genomic locus, it is important that the sgRNA targeting occurs as near to the insertion site as possible. Below details the strategy to create a C-terminal in-frame GFP knock-in, however this protocol can be adapted to knock in any insert at any locus following this sgRNA and homology-sequence design. Efficiencies reported here are representative for ±1-2 kb sequences. Larger insertions are feasible if they can be PCR amplified, and they may have less efficient knock-in. Homologous recombination (HR) replacement of short regions such as SNP repair is possible using this protocol and facilitates knock-in in 20-50% of cells.

Materials
sgRNA oligos
Proofreading PCR polymerase (NEBNext)
Agarose
Gel electrophoresis unit
Minelute DNA purification kit (or standard DNA purification followed by vacuum concentration)
sgPal7-HygR plasmid maxiprep DNA (Addgene #71484)
CBH-Cas9-BlastR plasmid maxiprep DNA (Addgene (#71489)
Knock-in template
Sterile eppendorff 1.5 ml microcentrifuge tubes
Cell electroporator and cuvettes
Cells and appropriate cell culture media and reagents
Y-27632 ROCK-inhibitor
Hygromycin
Blasticidin
Purelink Genomic DNA Mini Kit
gDNA Lysis Buffer (10 mM TrisHCl (pH7.5 or pH 8.0), 10 mM EDTA, 10 mM NaCl, 0.5%
SDS) with 1 mg/ml Proteinase K added fresh
   Step 1—scCRISPR sgRNA and Insertion Homology-Arm Design

1. Use a genome browser, such as the UCSC genome browser, to identify the genomic sequence surrounding the gene of interest. If you are interested in making a C-terminal GFP fusion construct, identify the stop codon of the transcript you would like to tag (typically the primary RefSeq transcript), and copy ˜500 bp of genomic DNA sequence centered on the stop codon. Make sure to copy the sequence in the orientation of the coding sequence which may require reverse complementing the entire sequence. Annotate the STOP-codon within the sequence, below is an example for the mouse Pou5f1 gene where the STOP-codon is in bold and underlined:

Pou5f1
CGAGTATGGTTCTGTAACCGGCGCCAGAAGGGCAAAAGATCAAGTATTGA
GTATTCCCAACGAGAAGAGTATGAGGCTACAGGGACACCTTTCCCAGGGG
GGGCTGTATCCTTTCCTCTGCCCCCAGGTCCCCACTTTGGCACCCCAGGC
TATGGAAGCCCCCACTTCACCACACTCTACTCAGTCCCTTTTCCTGAGGG
CGAGGCCTTTCCCTCTGTTCCCGTCACTGCTCTGGGCTCTCCCATGCATT
CAAACTGAGGCACCAGCCCTCCCTGGGGATGCTGTGAGCCAAGGCAAGGG
AGGTAGACAAGAGAACCTGGAGCTTTGGGGTTAAATTCTTTTACTGAGGA
GGGATTAAAAGCACAACAGGGGTGGGGGGTGGGATGGGGAAAGAAGCTCA
GTGATGCTGTTGATCAGGAGCCTGGCCTGTCTGTCACTCATCATTTTGTT
CTTAAATAAAGACTGGGACACACAGTAGATAGCTGAATTTTGTTTTCCTT
CAG

2. In order to make a C-terminal fusion protein, find the NGG on either strand that is closest to the stop codon, as homologous recombination works best when the homology arms are as close as possible to the double-strand break. Note that the sgRNA will continue to cleave any sequence with significant similarity to the target sequence, so it is pertinent to ensure the homology sequences used do not overlap with the protospacer sequence. It is best to avoid overlap between the homology sequence and sgRNA of more than 5 bp, which means that a portion of the sgRNA sequence will be removed from the genome. To avoid loss of protein coding sequence, NGG sequences prior to or containing the stop codon should not be used, but NGG sequences after the stop codon and CCN sequences immediately prior to, including, or after the stop codon can be used.

Keeping this in mind, the targeting sgRNA is designed as described above, as near to the insertion site as possible while avoiding the coding region. The sgRNA was designed in the reverse complement, depicted above italicized. Note that the GGG and CCC PAM sequences prior to the stop codon and the AGG abutting the stop codon were not used because too much of their sgRNA sequence must be retained in the homology construct, so the sgRNA would cut in the homology construct and prevent its integration. The PAM sequence used was thus the closest acceptable PAM sequence to the stop codon, denoted above in italics.

Note: If no appropriate sgRNA sequence can be found outside of the coding region, or if it is desired to knock-in the insert within a coding region, the sgRNA recognition sequence will necessarily overlap with the homology-arms. In this case, to maintain the protein-coding sequence it is recommended to design the area of the homology-arms that overlaps with the sgRNA to have silent mutations, such that these codons remain unchanged, while the DNA sequences are significantly modified so that they are no longer recognized by the targeting sgRNA. The PAM sequence is the most important to Cas9 cutting, so ablating the PAM sequence from the desired homologous recombination-repaired genotype is the best way to avoid cutting.

TABLE 9

| | |
|---|---|
| Pou5f1_g RNA | 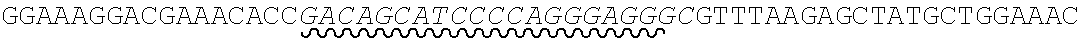 |

3. Homology arms are designed to encode GFP in-frame immediately upstream of the stop codon of the gene and to include a stop codon after the GFP open reading frame (ORF). Homology arms were designed so as not to overlap with the gRNA sequence by more than 5 bp on either side. For the forward arm, the homology arm should always include the entire last codon before the stop codon to allow in-frame C-terminal GFP fusion. For the reverse arm, the rule of not overlapping with the gRNA sequence by more than 5 bp means that a portion of the 3′ UTR will be removed. This does not appear to be a problem for gene expression, but the extent of this removal is limited by designing gRNAs as close to the stop codon as possible (see above section) and designing homology arm primers with the maximum allowable 5 bp of overlap with the gRNA. Left and right homology sequences are depicted above, underlined.

4. Design primers to PCR amplify the insertion cassette with homology arms for the insertion locus. To insert GFP, one can use the bolded primer sequences below. This GFP sequence can be ordered as a gBlock to use as PCR template for the knock-in cassette. Note the GFP STOP-codon at the 3′-end (bolded and double-underlined), and take care to remove this sequence when necessary. Genomic homology sequences are attached upstream of these primers to create the flanking homology arms in two consecutive PCR steps using 60 bp oligos. The first primer pair is of the format in the table below, adding the 40 bp of genomic homology sequence nearest to the insertion site, to each primer. The second homology-arm primer pair overlaps with the first homology arms by 20-30 bp. A minimal overlap is chosen such that the overlapping region is estimated to have a Tm of >65 degrees Celsius using the NEB Tm calculator. To ensure efficient PCR it is preferred that one does not use less than 20 bp of overlap. Then, they are extended by 30-40 bp on each end up to 60 bp maximum. Examples of homology-arm primers for Pou5f1 knock-in of GFP, based on the homology sequence denoted above, can be found in the table below:

GFP
GTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCG
AGGGCGATGCCACCTACGGCAAGCTGACCCTGAAGTTCATCTGCACCACC
GGCAAGCTGCCCGTGCCCTGGCCCACCCTCGTGACCACCCTGACCTACGG
CGTGCAGTGCTTCAGCCGCTACCCCGACCACATGAAGCAGCACGACTTCT
TCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGA
CACCCTGGTGAACCGCATCGAGCTGAAGGGCATCGACTTCAAGGAGGACG
GCAACATCCTGGGGCACAAGCTGGAGTACAACTACAACAGCCACAACGTC
TATATCATGGCCGACAAGCAGAAGAACGGCATCAAGGTGAACTTCAAGAT
CCGCCACAACATCGAGGACGGCAGCGTGCAGCTCGCCGACCACTACCAGC
AGAACACCCCCATCGGCGACGGCCCCGTGCTGCTGCCCGACAACCACTAC
CTGAGCACCCAGTCCGCCCTGAGCAAAGACCCCAACGAGAAGCGCGATCA
CATGGTCCTGCTGGAGTTCGTGACCGCCGCCGGGATCACTCTCGGCATGG
ACGAGCTGTACAAGTAAAGCGGCCGCAATTCACTCCTCA

TABLE 10

| Design | Primer | Sequence |
|---|---|---|
| (Pre-STOP 40bp) - GFP_Fw | GFPstep1_Fw | TGTTCCCGTCACTGCTCTGGGCTCTCCCATGCATTCAAAC |
| | | GTGAGCAAGGGCGAGGAGCT |
| (Post-STOP 40bp) - GFP_Rv | GFPstep1_Rv | CCAGGTTCTCTTGTCTACCTCCCTTGCCTTGGCTCACAGC |
| | | TGAGGAGTGAATTGCGGCCG |
| (35-40bp ext) - GFP_step1_Fw | GFPstep2_Fw | TCTACTCAGTCCCTTTTCCTGAGGGCGAGGCCTTTCCCTC |
| | | TGTTCCCGTCACTGCTCTGG |
| (35-40bp ext) - GFP_step1_Rv | GFPstep2_Rv | TAATCCCTCCTCAGTAAAAGAATTTAACCCCAAAGCT |
| | | CCAGGTTCTCTTGTCTACCTCCC |

5. Finally, genomic DNA PCR primers were designed to verify whether site specific homologous recombination was successful, using e.g., Primer3 with standard settings. Primers must be outside of the amplified homology arms to avoid background of unintegrated homology arm construct. To do this, paste the 500 bp genomic sequence into Primer 3 placing “[” and “]” at the end of the homology arms. Primer3 will give you one forward primer before the homology arm and one reverse primer after the homology arm that can typically be paired with the GFP primers below to look for locus-specific GFP integration. Examples for Pou5f1 flanking primers are also listed below:

TABLE 11
Exemplary Pou4f1 flanking primers

| | |
|---|---|
| GFPearly_Rv | GTCCAGCTCGACCAGGATG |
| GFPlate_Fw | GGATCACTCTCGGCATGGAC |
| Pou5f1insert_Fw | TGTATCCTTTCCTCTGCCCC |
| Pou5f1insert_Rv | GAGCTTCTTTCCCCATCCCA |

### Step 2—Homology Insertion Cassette PCR Amplification

The protocol below is for high-fidelity amplification with 2×NEBNext Mastermix, however other proofreading polymerases can also be used for faithful gene knock-in. The insertion cassette is PCR-amplified in two successive PCR steps to generate a total of ±150 bp of flanking homology sequence to the insertion site. PCR step 1 is run with 15 amplification cycles to save time, while PCR step 2 is run with 35 cycles to generate a large number of amplicons. The sgRNA-HDR oligo used for targeting is PCR amplified and prepared as explained in the protocol above with one minor modification: that the final PCR step 3 is scaled to a 200 ul volume.

6. PCR step 1 on GFP template with GFPstep 1 forward and reverse primers using NEBNext polymerase (Ta=60-72° C., typically 72° C.), and run for 15 amplification cycles. Amplification product is 819 bp. PCR mix below is for typical 20 ul reaction volume, but can be scaled if needed.

TABLE 12
Exemplary reaction mix for Step 1 of homology
insertion cassette PCR amplification

| | | |
|---|---|---|
| | | % Reaction |
| Volume | Reagent | Volume |

| | | | |
|---|---|---|---|
| 10 | ul | NEBNext 2x Master Mix | 50% |
| 0.1 | ul | 100 ng/ul stock of GFP template | 0.5% |
| 0.5 | ul | 20 uM GFPstep1 Fw primer | 2.5% |
| 0.5 | ul | 20 uM GFPstep1 Rv primer | 2.5% |
| 0.6 | ul | DMSO | 3% |
| 8.3 | ul | mQ water | 41.5% |

7. PCR step 2 on 819 bp amplification product of PCR step 1, with HDRstep3 forward and reverse primers using NEBNext polymerase (Ta=60-72° C., typically 72° C.), and run for 30 amplification cycles. Amplification product of PCR step 1 is used directly without purification, and it is unnecessary to determine DNA concentration. If desired, amplicon size can be validated by running a small aliquot of PCR step 1 on a 2% agarose by gel electrophoresis as described below in step of this protocol, though this is not necessary at this stage. Amplification product is ±900 bp. PCR mix below is for typical 200 ul reaction volume, but can be scaled if needed.

TABLE 13
Exemplary reaction mix for Step 2 of homology
insertion cassette PCR amplification

| | | |
|---|---|---|
| | | % Reaction |
| Volume | Reagent | Volume |

| | | | |
|---|---|---|---|
| 100 | ul | NEBNext 2x Master Mix | 50% |
| 10 | ul | Unpurified product of PCR 1 | 0.5% |
| 5 | ul | 20 uM GFPstep2 Fw primer | 2.5% |
| 5 | ul | 20 uM GFPstep2 Rv primer | 2.5% |
| 6 | ul | DMSO | 3% |
| 74 | ul | mQ water | 37% |

8. Final amplicon is validated by gel electrophoresis, running a 2 ul aliquot on a 2% agarose gel. Product is as follows:

GFP primers PCRstep1 homology primers PCRstep 2
homology primers
TCTACTCAGTCCCTTTTCCTGAGGGCGAGGCCTTTCCCTCTGTTCCCGTC
ACTGCTCTGGGCTCTCCCATGCATTCAAACGTGAGCAAGGGCGAGGAGCT
GTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTG
GCCCACCCTCGTGACCACCCTGACCTACGGCGTGCAGTGCTTCAGCCGCT
ACCCCGACCACATGAAGCAGCACGACTTCTTCAAGTCCGCCATGCCCGAA
GGCTACGTCCAGGAGCGCACCATCTTCTTCAAGGACGACGGCAACTACAA
GACCCGCGCCGAGGTGAAGTTCGAGGGCGACACCCTGGTGAACCGCATCG
AGCTGAAGGGCATCGACTTCAAGGAGGACGGCAACATCCTGGGGCACAAG
CTGGAGTACAACTACAACAGCCACAACGTCTATATCATGGCCGACAAGCA
GAAGAACGGCATCAAGGTGAACTTCAAGATCCGCCACAACATCGAGGACG
GCAGCGTGCAGCTCGCCGACCACTACCAGCAGAACACCCCCATCGGCGAC
GGCCCCGTGCTGCTGCCCGACAACCACTACCTGAGCACCCAGTCCGCCCT
GAGCAAAGACCCCAACGAGAAGCGCGATCACATGGTCCTGCTGGAGTTCG
TGACCGCCGCCGGGATCACTCTCGGCATGGACGAGCTGTACAAGTAAAGC
GGCCGCAATTCACTCCTCAGCTGTGAGCCAAGGCAAGGGAGGTAGACAAG
AGAACCTGGAGCTTTGGGGTTAAATTCTTTTACTGAGGAGGGATTA

9. There is no need to gel purify unless there is an abundance of smaller band. Minelute PCR purify the remaining 198 ul of PCR step 2 in 10 ul.

Step 3—scCRISPR Cell Targeting for HR

Efficient cell targeting is achieved by electroporation as described below, or slightly lower efficiency by nucleofection. Transfection yields low knock-in efficiency and is not recommended.

For Electroporation

10. For electroporation into 6-well, combine the 10 ul eluate of the 200 ul Minelute sgRNA PCRstep3, and the 10 ul eluate of the 200 ul Minelute insertion cassette PCRstep2 from above with 5 ug CBH-Cas9-BlastR plasmid and 5 ug sgPal7-HygR plasmid. If total volume exceeds 20 ul, concentrate by vacuum centrifugation until it is reduced below this threshold: be vigilant so that the DNA mix does not get contaminated in this process and the DNA does not dry out.

11. Prepare single cell suspension by enzymatic passaging and concentrate cells by centrifugation for 5 minutes at 200 g.

12. Transfer appropriate number of cells to electroporation cuvette in 120 ul EmbryoMax Electroporation buffer and gently mix well with DNA. For mouse and human embryonic stem cells (ESCs), use ±10⁶ cells and 0.4 cm electroporation cuvettes.

13. Electroporate cells with appropriate settings for the cell type used. For mouse and human ESCs electroporate at 230 V, 0.500 mF, and maximum resistance.

14. Plate in 6-well and incubate at 37° C. overnight. Mouse and human ESCs in complete ESC media can be plated with added 7.5 uM Y-27632 if desired, to help recovery and reduce cell death following electroporation.

15. Select with Blasticidin and Hygromycin from hours 24-72 after targeting. For mouse ESCs, select with 10 ug/mL Blasticidin+100 ug/mL Hygromycin. For human ESCs, select with 2 ug/mL Blasticidin+66 ug/mL Hygromycin.

16. Replace selection media with regular media and allow cells to recover and expand for ±7 days after electroporation. When passaging, take sample to analyze targeting efficiency as described below. Expect 2-4% insertion efficiency by electroporation in ESCs and >10% for HEK293T cells. Representative data for mESC knock-in efficiency is shown in FIG. 1c, by C-terminal knock-in of GFP in the Pou5f1 gene.

### Step 4—Analyzing HR Knock-in Efficiency

Validating knock-in can be done in various ways, depending on the knock-in cassette. Insertion of a fluorescent, or antibiotic-resistance reporter of a gene that is expressed in the target cell type, may allow for swift selection of target clones by flow-cytometric sorting, or antibiotic selection of successfully targeted clones. Still, subsequent sequence verification of clones may still be recommended in these cases, or when phenotypic selection is not possible.

Described below is a method to determine knock-in efficiency by PCR of bulk, and clonal targeted cells. For fastest identification of positive clones, simultaneously plate cells for 96-well clonal analysis, while performing bulk population PCR validation, in the protocols below.

Bulk Population Genomic DNA Analysis

17. When splitting confluent 6-well of targeted cells, collect genomic DNA on ˜½ of cells using the Purelink Genomic DNA Mini Kit.

18. Use the isolated bulk genomic DNA to check for integration using the primers ordered for this purpose during homology construct generation. There are three possible primer pairs to use that will confirm the left-end, right-end, and complete insertion size. All three can, and should be used to confirm insertion, but keep in mind that Pair 3 to check for insertion size will mainly show the wildtype locus in a bulk population in which knock-in genotypes are the minority, as this amplicon is smaller and will dominate the PCR. For the example of GFP insertion into the Pou5f1 C-terminus, one can use the following primer combinations:

TABLE 14
Primer combination examples

| Pair | Forward Primer | Reverse Primer |
|---|---|---|
| 1 | Pou5f1insert_Fw | GFPearly_Rv |
| 2 | GFPlate_Fw | Pou5f1insert_Rv |
| 3 | Pou5f1insert_Fw | Pou5f1insert_Rv |

PCR using the above pairs on 200 ng of bulk isolated genomic DNA (gDNA) using Onetaq polymerase (Typically Ta=60° C.), and run for 35 amplification cycles. PCR mix below is for typical 20 ul reaction volume.

TABLE 15
Exemplary PCR reaction mix to confirm insertion

| | | | % Reaction |
|---|---|---|---|
| Volume | | Reagent | Volume |
| 10 | ul | OneTaq 2x Master Mix with standard buffer | 50% |
| x.x | ul | 200 ng Bulk gDNA | |
| 0.5 | ul | 20 uM Validation primer Fw | 2.5% |
| 0.5 | ul | 20 uM Validation primer Rv | 2.5% |
| x.x | ul | mQ water | up to 100% |

Check amplicon by gel electrophoresis, by running sample on a 2% agarose gel. If the bands are absent, weak, or have competing bands at incorrect sizes, optimize the Ta of this PCR and try PCR conditions both with, and without 3% DMSO. This step is important not only to ensure that GFP has integrated in the population but also to optimize primer combinations, as the 96-well genomic DNA PCRs are messier so require robust primers.

19. Optionally, qPCR can be used, after successful PCR conditions have been found, to estimate integration frequency. To do so, primer pairs that only give a product after correct integration should be used along with genomic DNA control primers that occur twice in every cell. By comparing amplification cycle number, approximate integration frequency can be established.

96-Well Genomic DNA Analysis of Feeder-Free ESCs

Note that at this stage, individual colonies can be picked, or limiting dilution can be performed to reduce the number of colonies that eventually have to be picked. Given the typical 2-4% knock-in efficiency for a 1-2 kb insert, picking 96 colonies is a recommended minimum. The protocol below is for limiting dilution followed by single colony picking or limiting dilution from positive wells.

20. Count the cells and plate 96 wells of a 96-well plate at ˜7 cells/well.

21. Once 96-wells are at least ⅓ confluent, prepare to split. To do so, prepare appropriate surface-coating for ESC type, and add 100 uL of media into wells.

22. Dissociate cells enzymatically using 25 ul of Trypsin/EDTA solution, breaking up clumps with a multi-channel pipette.

23. Transfer half of the cell solution into the new 96-well plate containing media to continue culture, and use the other half for 96-well gDNA isolation.

24. Add 50 uL genomic DNA lysis buffer with added Proteinase K.

25. Seal plate using parafilm and place plate in humidified staining chamber to avoid evaporation, then place chamber at 60 degrees. This step is ideally done overnight but can be shortened to >3 hrs.

26. Carefully add 100 uL/well of pre-chilled 100% EtOH+75 mM NaCl (add NaCl fresh as it does not really go into solution-150 uL 5 M NaCl per 10 mL EtOH) to each well and let sit on benchtop at room temp for 30 min.

27. Carefully invert plate to remove liquid then add 150 uL/well 70% EtOH. Invert and repeat for 2 total 70% EtOH washes.

28. After second wash, invert and then shake plate vigorously to remove all EtOH and blot upside down on paper towels to remove all EtOH. Shake vigorously and blot every few minutes, and let plate dry for 10 min at room temp

29. Add 30 uL/well TE Buffer/Elution Buffer and allow to dissolve. Optionally place back at 60 degrees for several minutes to facilitate DNA dissolving. Prepare PCR mixture for Onetaq PCR reactions of each colony, according to optimized PCR conditions determined by bulk population gDNA analysis. Typically, a 15 ul reaction volume is used and run for 35 amplification cycles.

30. Note which wells give positive PCR reactions. As these reactions were isolated from 7 clones per well originally, these wells must be further subcloned by limiting dilution or by colony picking, and new clones should again be verified by PCR. Clones should always be verified through complete sequencing using locus forward and reverse primers, as it is possible to get partial insertions or insertions with mutations.

### Further Information

Genomic engineering is an invaluable tool in discerning the role of DNA and gene products in cell function¹⁻³. Since the initial reports describing how CRISPR/Cas9 could be modified to target specific genomic loci, this technology has greatly impacted the course of genetic and cellular research. Design of site-specific targeting sgRNAs is much improved from the laborious construction of recombinant endonucleases, such as TALENs and ZFNs^{6,7}, which makes CRISPR a far more amenable genome editing system.

Still, the requirement to clone novel sgRNA plasmids for each locus is disproportionately time consuming compared to the ease with which sgRNAs are designed and CRISPR cell targeting can be accomplished. Currently, sgRNA plasmid cloning can take up the same amount of time as targeting and validating correctly targeted cells. One can bypass the lengthy, and relatively costly step of sgRNA plasmid construction by allowing the sgRNA plasmid recombination, normally achieved by molecular cloning, to occur within cells at the same time as genome targeting, without needing to compromise on targeting efficiency.

Similarly, the challenging steps involved in homology plasmid construction hold back CRISPR-mediated transgenesis from becoming a routine application. Presently, genomic insertion of gene sequences, such as to create reporters of gene expression, are reserved for only the most essential experiments due to the effort involved. Using the minimal parameters explained in this protocol, to add short ±80 bp long flanking homology regions onto the insertion sequence, the time, difficulty and cost of homology-mediated gene insertion are overcome.

Cloning-free approaches further improve on CRISPR engineering technology by avoiding the few demanding steps involved in the locus-specific gene editing process, to facilitate genomic modification and transgenesis of in vitro models and help to accelerate the progress of molecular research.

Parameters to Keep in Mind

Good tissue culture practice and sterile technique are important to maintain healthy cell cultures and avoid contamination. When preparing single cell solutions for electroporation, care should be taken to treat cells gently so as to avoid unnecessary additional stress, as harsh treatment may lead to an increase in cell death and lower targeting efficiency. Limiting the volume of the DNA mix used for electroporation is also important to maintain the correct balance of the components in the electroporation solution. As described above, the cells are electroporated in 120 ul of electroporation buffer and restrict the DNA mix volume to 20 ul or less.

Depending on the cell line used, the antibiotic concentrations may need to be adjusted to ensure optimal selection. If excessive cell death is observed, even after optimization of antibiotic concentrations, selection could be limited to only one antibiotic instead of two. If so, we find single selection by blasticidin to be more effective than hygromycin incubation.

Use of other sgRNA and Cas9 expressing plasmids than the ones used in this work may yield a different targeting efficiency. Note, that dual-expression plasmids that encode both the sgRNA and Cas9 will lead to lower targeting efficiency than when these components are expressed from two separate plasmids.

The inventors recommend the palindromic sgRNA sgPal7 (#71484) and CBH-Cas9 (#71489) plasmids available through Addgene that have been extensively validated for this protocol.

Anticipated Results

Using the scCRISPR techniques described above, mutational targeting efficiencies of >90% in ESCs and HEK293T cells are routinely achieved. Knock-in efficiency is somewhat variable per locus, and depending on the size of the gene insert. From the +20 GFP reporter cell lines generated, it is predicted that ˜2-4% of cells will correctly insert the knock-in cassette in mouse and human ESCs. Efficiency of >10% knock-in is routinely observed in HEK293T cells

Time Considerations

With scCRISPR, cell targeting for NHEJ as well as gene insertion by HR can be done in under 2 hours after receiving the designed oligonucleotides for PCR amplification, by following the successive PCR steps described in the protocols above. This is a massive advance over the standard required cloning time of roughly a week. After targeting, transient antibiotic selection from 24-72 hours after electroporation improves the percentage of targeted cells in the bulk population. We will usually allow cells to recover and expand for another one or two passages before utilizing these cells for further experimentation. In all, an NHEJ-targeted cell line can be generated in approximately 1 week. Knock-in lines can usually be validated and expanded for subsequent use in roughly 2-3 weeks from initial cell targeting.

1. A method of generating a plasmid intracellularly for targeted modification of a genomic sequence, the method comprising introducing to the cell:
(a) an expression construct encoding an RNA-guided endonuclease; and
(b) a plasmid encoding a sequence directing the transcription of a self-targeting RNA guide sequence, comprising a self-targeting sequence, wherein the self-targeting RNA forms a complex with the RNA-guided endonuclease to initiate cleavage of a self-targeted sequence in the plasmid sequence encoding the self-targeting RNA guide sequence, such that transcription of the self-targeting RNA in the presence of the RNA-guided endonuclease permits the formation of a complex with the RNA guided endonuclease that directs the cleavage of the plasmid within the self-targeted sequence; and
(c) a repair template comprising a genomic targeting sequence flanked by first and second homology arms homologous, respectively, to sequences that flank said self-targeting sequence in the plasmid, the genomic targeting sequence sufficient to direct cleavage of an associated RNA-guided nuclease to a genomic target sequence,
wherein, upon introduction of the expression construct encoding an RNA-guided endonuclease, the plasmid and the repair template to the cell, the plasmid is cleaved in the self-targeted sequence and the repair template comprising the genomic targeting sequence directs the homologous replacement of the self-targeted sequence with the genomic targeting sequence, whereby the cleavage-guiding specificity of the self-targeted guide RNA is modified to the genomic target sequence.

2. The method of claim 1, wherein expressed RNA-guided endonuclease forms a complex with the modified guide RNA expressed from the plasmid and wherein the complex with modified guide RNA effects targeted modification of the genomic target sequence.

3. The method of claim 1, wherein the expression construct is a plasmid.

4. The method of claim 1, wherein the endonuclease is a Cas endonuclease.

5.-6. (canceled)

7. The method of claim 2, wherein the method does not require cloning of a sequence into a cloning vector.

8. The method of claim 2, further comprising providing a linear repair template for homologous recombination-mediated repair at the selected genomic target sequence.

9. The method of claim 8, wherein the process of homologous recombination inactivates the target sequence.

10. (canceled)

11. The method of claim 8, wherein the repair template comprises a sequence encoding one or more nucleotide mutation(s), one or more inserted nucleotide(s), or one or more deleted nucleotide(s).

12. (canceled)

13. The method of claim 1, wherein each of the self-targeted guide RNA sequence and the guide RNA expressed from the modified plasmid comprises a crRNA and/or a tracrRNA sequence to permit association of the guide RNA with the RNA-guided endonuclease.

14.-16. (canceled)

17. The method of claim 1, wherein the expression construct or the plasmid further comprises a sequence encoding a reporter molecule.

18. (canceled)

19. The method of claim 1, wherein the self-targeting sequence comprises a palindromic sequence.

20. A composition comprising a nucleic acid vector encoding a sequence directing the transcription of a self-targeting RNA guide molecule for an RNA-guided endonuclease,
the sequence comprising a self-targeting sequence, wherein when contacted with the RNA guided endonuclease, self-targeting RNA guide molecule transcribed from the vector forms a complex with the RNA-guided endonuclease, and
wherein the complex cleaves the plasmid in the sequence encoding the self-targeting RNA guide molecule, such that transcription of the self-targeting RNA in the presence of the RNA-guided endonuclease results in cleavage of the nucleic acid vector in the sequence encoding the self-targeting RNA guide molecule.

21. The composition of claim 20, wherein the nucleic acid vector further encodes an RNA-guided endonuclease.

22. The composition of claim 20, wherein the endonuclease is a Cas endonuclease.

23.-25. (canceled)

26. The composition of claim 20, wherein the self-targeting RNA guide molecule comprises a crRNA and/or a tracrRNA sequence to permit association of the guide RNA with the RNA-guided endonuclease.

27.-28. (canceled)

29. The composition of claim 20, wherein the self-targeting sequence comprises a palindromic sequence.

30. A composition comprising the composition of claim 20 and a linear repair template comprising a genomic targeting sequence, flanked by first and second homology arms homologous, respectively, to sequences that flank the self-targeting sequence in the vector.

31. A kit comprising the composition of claim 20 and instructions therefor.

32. The kit of claim 31, further comprising an expression construct encoding an RNA-guided endonuclease.

33. A cell comprising a composition of claim 20.

34.-36. (canceled)
